(19) United States
(12) Patent Application Publication
(10) Pub. No.: US 2022/0040318 A1
(43) Pub. Date: Feb. 10, 2022

(54) PRIMARY AMINE COMPOUND OR SECONDARY AMINE COMPOUND-ACIDIC POLYSACCHARIDE CONJUGATE AND PRODUCTION METHOD THEREFOR

(71) Applicant: Seikagaku Corporation, Tokyo (JP)

(72) Inventors: Nobuo Kobayashi, Tokyo (JP); Ryogo Hishiki, Tokyo (JP); Kenichi Namatsu, Tokyo (JP)

(73) Assignee: Seikagaku Corporation, Tokyo (JP)

(21) Appl. No.: 17/281,224

(22) PCT Filed: Sep. 27, 2019
(86) PCT No.: PCT/JP2019/038368

§ 371 Date: Oct. 19, 2021

(30) Foreign Application Priority Data
Sep. 28, 2018 (JP) 2018-185155

(51) Int. Cl. A61K 47/61; C08B 37/00; C08B 15/06; A61K 47/54

(52) U.S. Cl. CPC A61K 47/61; C08B 37/0069; A61K 47/54; C08B 37/0084; C08B 15/06

(57) ABSTRACT

Provided is a novel conjugate of a primary or secondary amine compound with an acidic polysaccharide, which is a compound represented by Formula (I) or a pharmaceutically acceptable salt thereof, where in Formula (I), D, R¹, R², A, and Poly are as defined in the specification.

### TECHNICAL FIELD

The present invention relates to a novel conjugate of a primary or secondary amine compound with an acidic polysaccharide and a production method therefore. Specifically, the present invention relates to a novel conjugate of a primary or secondary amine compound with an acidic polysaccharide using, as a linker, an aminoacyloxymethyl group whose release rate can be controlled, and a production method therefore.

### BACKGROUND ART

A conjugate of a drug with a polymer has been widely reviewed in a field of a prodrug or drug delivery system (DDS), and is an important means for providing a function such as release control, absorption improvement, stabilization in a living body, or targeting to a target tissue.

For example, a conjugate of a water-soluble non-polypeptide oligomer and calcimimetics have been reported in US 2012/238621 A. Further, a conjugate of sodium carboxymethyl cellulose (CMC) with gossypol has been reported in JP 5690944 B2. Alginic acid, which is one of dietary fibers, has also been reviewed as a polymer used for the conjugate, and conjugates of alginic acid and various drugs have been reported in JP H08-24325 A. Further, glycosaminoglycan (GAG) has also been widely reviewed as the polymer used for the conjugate, and a conjugate of hyaluronic acid or chondroitin sulfate with a peptide has been reported in U.S. Pat. No. 5,955,578. Furthermore, conjugates of heparin with various drugs have been reported in WO 93/18793 A. Furthermore, hyaluronic acid has also been reviewed as the polymer used for the conjugate, a conjugate of hyaluronic acid with taxane has been reported in WO 2005/085294 A, and a conjugate of hyaluronic acid with a protein such as a serine protease inhibitor has also been reported in JP 2006-504747 A.

Further, a method of bonding GAG and a primary amine drug by reductive amination has been reported in JP 2000-501082 A, and a method of forming an amide bond between a primary or secondary amine drug and an acidic polysaccharide which is a polysaccharide having a carboxy group in a monomer structure has been reported in JP H08-24325 A.

### CITATION LIST

### Patent Literature

Patent Literature 1: US 2012/238621 A
Patent Literature 2: JP 5690944 B2
Patent Literature 3: JP H08-24325 A
Patent Literature 4: U.S. Pat. No. 5,955,578
Patent Literature 5: WO 93/18793 A
Patent Literature 6: WO 2005/085294 A
Patent Literature 7: JP 2006-504747 A
Patent Literature 8: JP 2000-501082 A
Patent Literature 9: WO 2012/088522 A
Patent Literature 10: JP 2003-511423 A

### Non Patent Literature

Non Patent Literature 1: J. Control. Rel. Vol. 88, pp. 35-42, 2003
Non Patent Literature 2: Expert Opin. Drug Deliv. Vol. 9, pp. 1319-1323, 2012
Non Patent Literature 3: Immunogenicity Assessment for Therapeutic Protein Products, 2014
Non Patent Literature 4: EMA/CHMP/SWP/647258/2012
Non Patent Literature 5: Lecture 2 “Surprising Allergen Causing Food Allergy” The 32nd Annual Meeting of Japan Organization of Clinical Dermatologists, 2016
Non Patent Literature 6: J. Med. Chem. Vol. 39, pp. 424-431, 1996
Non Patent Literature 7: Anesthesia & Analgesia, Vol. 105, pp. 724-728, 2007
Non Patent Literature 8: Int. J. Nanomed. Vol. 7, pp. 2957-2966, 2012

### SUMMARY OF INVENTION

As described above, various drug-polymer conjugates have been studied. Polyethylene glycol (PEG) is one of the widely used water-soluble polymers, and a conjugate of PEG having a functional group introduced into the terminal of a linear molecule with a drug has been reported in WO 2012/088522 A and US 2012/238621 A. Further, a conjugate using poly-γ-glutamic acid (PGA), which is one of the polyamino acids, has been reported in JP 2003-511423 A.

In the case of using PEG or PGA as a polymer for these conjugates, pharmacokinetic or safety concerns have been reported. For example, the accelerated blood clearance phenomenon (ABC phenomenon), in which an anti-PEG antibody is generated by administration of PEG formulation, and the clearance at the time of the second or later administration is accelerated, has been reported in J. Control. Rel. Vol. 88, pp. 35-42, 2003 and Expert Opin. Drug Deliv. Vol. 9, pp. 1319-1323, 2012. Further, the U.S. Food and Drug Administration (FDA) recommends the measurement of the anti-PEG antibody, in addition to the measurement of anti-protein drug antibodies, in clinical trials of PEGylated protein formulation (Immunogenicity Assessment for Therapeutic Protein Products, 2014). Furthermore, vacuolation of macrophages and ependymal cells due to repeated administration of PEGylated formulation of 40 kDa or more has been observed (EMA/CHMP/SWP/647258/2012). Regarding PGA, which is also an antigenic substance of natto allergy, cases of delayed anaphylactic shock have been reported (Lecture 2 “Surprising Allergen Causing Food Allergy” The 32nd Annual Meeting of Japan Organization of Clinical Dermatologists, 2016).

In the case of considering conjugates as medical drugs, it is necessary to take into account not only safety but also drug effect onset, solubility, sustained release performance, or the like. Hydrolysis is generally used as a mechanism by which a prodrug releases a drug. In the case of a drug having a carboxy group or a hydroxyl group in the molecule, an ester bond can be formed with the acidic polysaccharide via a linker, and the drug can be released by hydrolysis of the ester bond. On the other hand, in the case of a primary or secondary amine drug, it is expected that the bond by reductive amination or the amide bond formed in the conjugate is not easily hydrolyzed, and thus does not have sufficient drug releasing ability. Regarding a primary or secondary amine drug having no carboxy group or hydroxyl group in the molecule, it is not possible to form an ester bond with a polymer via a linker using a method of the related art. Accordingly, there are no acidic polysaccharide conjugates in which drug release control is achieved using hydrolyzation, as the basic mechanism of prodrugs.

Since conjugation reaction is selected depending on a functional group of a drug, a conjugate of a primary or secondary amine compound having a sustained-release property with an acidic polysaccharide cannot be obtained by a method of the conventional art, and thus construction of a novel method has been desired.

In addition, it has been reported that the polymer molecular weight of the conjugate is closely related to its performance as DDS formulation, and the retentivity in the tissue or blood as well as the rate of absorption from the digestive tract of the conjugate are improved depending on the size of the polymer molecular weight (J. Med. Chem. Vol. 39, pp. 424-431, 1996; Anesthesia & Analgesia, Vol. 105, pp. 724-728, 2007; Int. Int. J. Nanomed. Vol. 7, pp. 2957-2966, 2012). However, in general, increasing the molecular weight of the polymer lowers the water solubility of the polymer itself. As a result, the water solubility of the conjugate using the high-molecular-weight polymer also decreases. As described above, increasing the molecular weight of the carrier for the purpose of improving retentivity in tissue or blood is not simple, and it is necessary to balance the molecular weight with the water solubility. In the case of using a polymer having a small number of reactive sites that binds to a drug such as PEG, the amount of drug introduced per polymer molecule is inevitably low. In the case of using a high-molecular weight form, the amount of a drug in a drug solution may be significantly reduced due to solubility, and may not reach the drug concentration required for the drug effect onset. Further, PEG can be said to be a disadvantageous carrier for increasing the molecular weight of the polymer because toxicity concerns increase when the molecular weight is 40 kDa or more, as described above.

An object of the present invention is to provide a novel conjugate that has a primary or secondary amine compound and includes a high drug concentration, high retentivity and solubility, and a production method therefor. Particularly, an object of the present invention is to provide a novel conjugate of a primary or secondary amine compound with an acidic polysaccharide, and a production method therefore.

The present inventors have conducted intensive studies on the problems of these conjugates that have not been achieved for primary or secondary amine drugs. As a result, they have found a novel conjugate of a primary or secondary amine compound with an acidic polysaccharide using, as a linker, an aminoacyloxymethyl group whose release rate can be controlled, and have completed the present invention. The present invention is a novel conjugate of a primary or secondary amine compound with an acidic polysaccharide and a production method therefore.

### BRIEF DESCRIPTION OF DRAWINGS

FIG. 1 is a graph showing a relation between time and a drug release ratio in a buffer solution having a pH of 7.0 in Examples 22, 23, 29, and 31.

FIG. 2 is a graph showing a relation between time and a drug release ratio in a buffer solution having a pH of 7.0 in Examples 34, 63, and 69.

FIG. 3 is a graph showing a relation between time and a drug release ratio in a buffer solution having a pH of 7.0 in Example 80.

FIG. 4 is a graph showing a relation between time and a drug release ratio in a buffer solution having a pH of 7.0 in Examples 36, 38, 41, and 47.

FIG. 5 is a graph showing a relation between time and a drug release ratio in a buffer solution having a pH of 7.0 in Examples 86, 88, and 89.

FIG. 6 is a graph showing the filterability of a 0.22 μm filter for Example 81 and Reference Example 1, based on a relation between a drug concentration and a solution passage rate.

FIG. 7 is a graph showing the filterability of a 0.22 μm filter for Example 82 and Reference Example 1, based on a relation between a drug concentration and a solution passage rate.

FIG. 8 is a graph showing the filterability of a 0.22 μm filter for Examples 84 and 85 and Reference Example 2, based on a relation between a drug concentration and a solution passage rate.

### DESCRIPTION OF EMBODIMENTS

A conjugate according to an aspect of the present invention is a compound having a structure represented by the following Formula (I) or a pharmaceutically acceptable salt thereof;

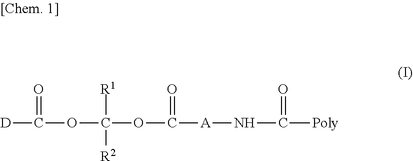

in Formula (I), D represents a residue of a primary or secondary amine compound DH excluding a hydrogen atom of a primary or secondary amino group; R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a substituted or unsubstituted bivalent hydrocarbon group and may contain one or more hetero atoms at a position except for both ends which are bonded to —C(═O)— or —NH—, the hetero atoms are each independently selected from the group consisting of —O—, —NH— which may have a substituent, and —S—; any two or three groups of R¹, R², and A may combine together to form a ring; Poly represents an acidic polysaccharide residue, and —C(═O)— adjacent to Poly is derived from a carboxy group of the acidic polysaccharide.

A structure of an acidic polysaccharide Poly-CO₂H excluding the OH moiety of the carboxy group used for condensation with the amine form, which is represented by Poly-CO, and a structure in which in a compound itself or a pharmaceutically acceptable salt thereof, a primary or secondary amine compound DH excludes a hydrogen atom of a primary or secondary amino group, which is represented by D, are bonded to each other via a linker containing A to form a conjugate. The conjugate is preferably a conjugate with a drug containing a primary or secondary amino group. Hereinafter, in the description of D, a primary or secondary amine or a pharmaceutically acceptable salt thereof may be collectively described as “primary or secondary amine compound”.

As mentioned above, increasing the molecular weight of the polymer as a partial structure of the conjugate is an important factor in considering the function of the DDS formulation. However, PEG is a carrier disadvantageous for increasing the molecular weight due to structural problems and toxicity concerns. On the other hand, in the case of using the acidic polysaccharide as a carrier of the conjugate, a pharmaceutical composition having a high drug carrying amount and few toxicity concerns can be prepared even if the molecular weight of the polymer is increased as shown in the test results described below.

The linker having the structure found in the present invention is capable of producing a novel primary or secondary amine compound-acidic polysaccharide conjugate. The conjugate uses an acidic polysaccharide as a carrier, so that it is possible to provide a highly practical pharmaceutical composition in terms of safety, polymer molecular weight, drug carrying amount, and the like, compared with a technique of the related art. This invention considerably contributes to medical treatment and the like.

The conjugate binds to a hydrocarbon chain (a bivalent hydrocarbon group represented by A in the Formula (I)) in the linker when the carboxy group of the acidic polysaccharide residue forms an amide bond.

The bivalent hydrocarbon group represented by A may be a carbon chain having carbon number of not less than 1 and may have a branched structure or a cyclic structure. In a case where the bivalent hydrocarbon group A has a branched structure, it is possible to have a structure in which, in the Formula (I), an atom other than a hydrogen atom is bonded to at least one point of the molecular chain connecting a minimum number of a carbonyl group and an NH group to which A is bonded. Examples of the branched structure include structures such as 2-methyl-1,3-propylenyl and 1-phenyl-1,3-propylenyl. In a case where a part of the bivalent hydrocarbon group A has a cyclic structure, the ring can be bonded to the remaining part of A at any two atoms of the ring which may be the same atoms. Examples of the cyclic structure include 1,2-phenylene, 1,4-cyclohexylene, and 1,1-cyclopropenylene. Further, the cyclic structure may combine together with another partial structure, particularly a structure represented by —C(R¹)(R²)— in Formula (I) to form a ring. The bivalent hydrocarbon group A may have a substituent at any position, and examples of the substituent include those exemplified as the groups of R³, R⁴, R⁵, and R⁶ as described above. A preferably represents a bivalent hydrocarbon group represented by C(R³)(R⁴)—(CH₂)ₗ—(C(R⁵)(R⁶))ₘ—(CH₂)ₙ as represented by the following Formula (II) (herein, R³, R⁴, R⁵, R⁶, l, m, and n are as defined above). From the viewpoint of ease of design and availability of raw materials, A preferably represents a linear or branched alkylene group having carbon number of 1 to 10, and the carbon number of A is further preferably 1 to 6.

The conjugate represented by Formula (I) is preferably a compound represented by the following Formula (II) or a pharmaceutically acceptable salt thereof;

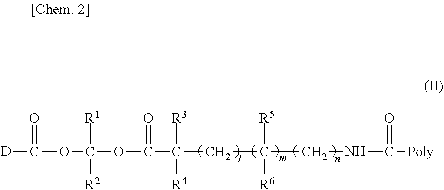

in Formula (II), D, R¹, R², and Poly are as defined above; R³, R⁴, R⁵, and R⁶ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three substituents of R¹, R², R³, R⁴, R⁵, and R⁶ may combine together to form a ring; 1 and n are each independently 0, 1, or 2, and m is 0 or 1.

In Formula (I), A is bonded to a substituted or unsubstituted methylene group represented by —C(R¹)(R²)— via an ester bond. On the other hand, a primary or secondary amine compound is bonded to the methylene group via a urethane bond. Accordingly, bonds are formed in the order of an oxygen atom of an ester bond-a carbon atom of the methylene group-an oxygen atom of a urethane bond-a carbon atom of a urethane bond—a nitrogen atom derived from the primary or secondary amine compound DH. The methylene group may be unsubstituted or substituted. In a case where the methylene group is substituted, two substituents may be combined together to form a ring, and may be bonded to a part of the bivalent hydrocarbon group A (i.e., at least one of R³, R⁴, R⁵, and R⁶ in Formula (II)) to form a ring. The ring may be a condensed ring or a spiro ring. The primary or secondary amine compound DH is present in the conjugate structure as urethane via a linker.

The urethane structure D-COO containing the primary or secondary amine compound DH at the terminal of the conjugate can rapidly decompose owing to the presence of an oxymethylene group to be bonded to the structure D-COO and can release the primary or secondary amine compound DH. This mechanism will be described using the compound represented by Formula (I) as follows. Regarding the primary or secondary amine compound-acidic polysaccharide conjugate represented by Formula (I), an ester bond moiety is hydrolyzed in the presence of water to be decomposed into a hydroxymethyl form represented by Formula (VII) and a carboxylic acid form represented by Formula (VIII). Further, the hydroxymethyl form represented by Formula (VII) is rapidly decomposed into the primary or secondary amine compound DH represented by Formula (IX), carbon dioxide represented by Formula (X), and an aldehyde form (or a ketone form) represented by Formula (XI), since it is structurally unstable due to having a urethane structure. At this time, the function of the generated primary or secondary amine compound is exhibited. Therefore, the primary or secondary amine compound-acidic polysaccharide conjugate represented by Formula (I) can control releasing of the primary or secondary amine compound by controlling a hydrolysis rate of the ester bond moiety, so that sustainability of the function of the primary or secondary amine compound can be controlled.

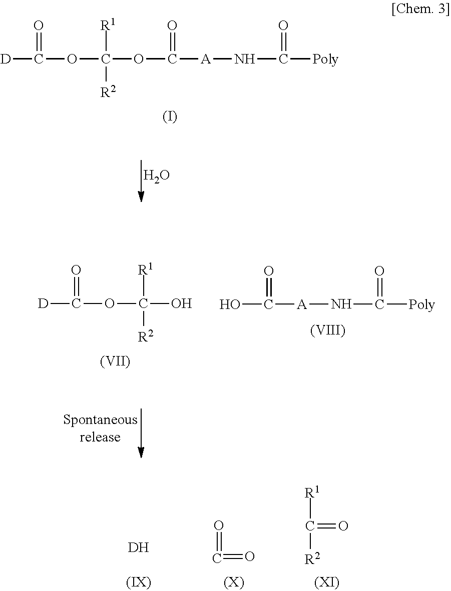

An aspect of the primary or secondary amine compound-acidic polysaccharide conjugate of the present invention is the compound represented by the Formula (I) or (II), and the amine form that is an important intermediate of the compound represented by (I) or (II) is a compound represented by the following Formula (III) or (XII):

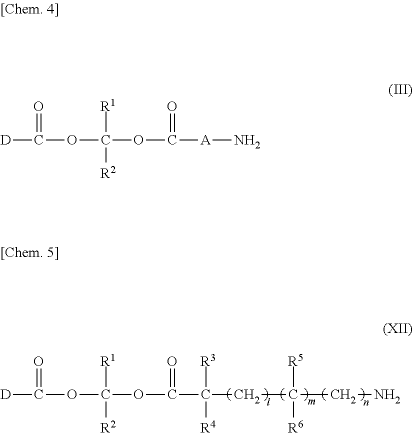

In the formula, D, R¹, R², R³, R⁴, R⁵, R⁶, A, l, m, and n are as defined above. The compound represented by the Formula (III) or (XII) may further form a salt with an inorganic acid or an organic acid.

Specific examples of the alkyl group, the cycloalkyl group, the alkenyl group, the cycloalkenyl group, the alkynyl group, the aromatic group, and the heterocyclic group included in the groups represented by the substituent R¹, R², R³, R⁴, R⁵, and R⁶ in Formulae (I), (II), (III), and (XII) include the following groups.

As the alkyl group, any of a linear or branched chain alkyl group may be used. The number of carbon atoms of the alkyl group is preferably 1, 2, 3, 4, 5, or 6. Examples of the alkyl group may include a methyl group, an ethyl group, an n-propyl group, a 2-propyl, an n-butyl group, a 1-methylpropyl group, a 1,1-dimethylethyl group, a 2-methylpropyl group, an n-pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, an n-hexyl group, a 1-methylpentyl group, a 2-methylpentyl group, a 3-methylpentyl group, a 4-methylpentyl group, a 1-ethylbutyl group, a 2-ethylbutyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 1,1,2-trimethylpropyl group, a 1-ethyl-1-methylpropyl group, and a 1-ethyl-2-methylpropyl group.

Any cycloalkyl group may be used as long as the carbon atom at the node is included as an atom constituting a ring, the cycloalkyl group may be condensed with cycloalkane, cycloalkene, an aromatic ring, or a hetero ring, and may form a spiro ring. The number of carbon atoms of the cycloalkyl group is preferably 3, 4, 5, 6, 7, or 8. Examples of the cycloalkyl group include a cyclopropyl group, a cyclobutyl group, a cyclopentyl group, a cyclohexyl group, a cycloheptyl group, and a cyclooctyl group.

As the alkenyl group, any of a linear or branched chain alkenyl group may be used. The number of carbon atoms of the alkenyl group is preferably 2, 3, 4, 5, or 6. Examples of the alkenyl group may include a vinyl group, a 1-propenyl group, a 2-propenyl group, a 1-methylvinyl group, a 1-butenyl group, a 2-butenyl group, a 3-butenyl group, a 1-ethylvinyl group, a 1-methyl-1-propenyl group, a 1-methyl-2-propenyl group, a 2-methyl-1-propenyl group, a 2-methyl-2-propenyl group, a 1-pentenyl group, a 2-pentenyl group, a 3-pentenyl group, a 4-pentenyl group, a 1-propylvinyl group, a 1-methyl-1-butenyl group, a 1-methyl-2-butenyl group, a 1-methyl-3-butenyl group, a 2-methyl-1-butenyl group, a 2-methyl-2-butenyl group, a 2-methyl-3-butenyl group, a 3-methyl-1-butenyl group, a 3-methyl-2-butenyl group, a 3-methyl-3-butenyl group, a 1-ethyl-1-propenyl group, a 1-ethyl-2-propenyl group, a 1-(2-methylethyl)vinyl group, a 1,2-dimethyl-1-propenyl group, a 1,2-dimethyl-2-propenyl group, a 1,1-dimethyl-2-propenyl group, a 1-hexenyl group, a 2-hexenyl group, a 3-hexenyl group, a 4-hexenyl group, a 5-hexenyl group, a 1-butyl vinyl group, a 1-methyl-1-pentenyl group, a 1-methyl-2-pentenyl group, a 1-methyl-3-pentenyl group, a 1-methyl-4-pentenyl group, a 2-methyl-1-pentenyl group, a 2-methyl-2-pentenyl group, a 2-methyl-3-pentenyl group, a 2-methyl-4-pentenyl group, a 3-methyl-1-pentenyl group, a 3-methyl-2-pentenyl group, a 3-methyl-3-pentenyl group, a 3-methyl-4-pentenyl group, a 4-methyl-1-pentenyl group, a 4-methyl-2-pentenyl group, a 4-methyl-3-pentenyl group, a 4-methyl-4-pentenyl group, a 1-propyl-1-propenyl group, a 1-propyl-2-propenyl group, a 1-ethyl-1-butenyl group, a 1-ethyl-2-butenyl group, a 1-ethyl-3-butenyl group, a 2-ethyl-1-butenyl group, a 2-ethyl-2-butenyl group, a 2-ethyl-3-butenyl group, a 1-(2-methylpropyl)vinyl group, a 1,2-dimethyl-1-butenyl group, a 1,2-dimethyl-2-butenyl group, a 1,2-dimethyl-3-butenyl group, a 1-(3-methylpropyl)vinyl group, a 1,3-dimethyl-1-butenyl group, a 1,3-dimethyl-2-butenyl group, a 1,3-dimethyl-3-butenyl group, a 2,3-dimethyl-1-butenyl group, a 2,3-dimethyl-2-butenyl group, a 2,3-dimethyl-3-butenyl group, a 3,3-dimethyl-1-butenyl group, a 2,2-dimethyl-3-butenyl group, a 1,1-dimethyl-2-butenyl group, a 1,1-dimethyl-3-butenyl group, a 1,1,2-trimethyl-2-propenyl group, a 1-ethyl-1-methyl-2-propenyl group, a 1-ethyl-2-methyl-1-propenyl group, a 1-ethyl-2-methyl-2-propenyl group, a 1-(1-methylethyl)-1-propenyl group, and a 1-(1-methylethyl)-2-propenyl group.

Any cycloalkenyl group may be used as long as the carbon atom at the node and a C═C double bond are included as an atom constituting a ring, the cycloalkenyl group may be condensed with a cycloalkane, a cycloalkene, an aromatic ring, or a hetero ring, and may form a spiro ring. The number of carbon atoms of the cycloalkenyl group is preferably 3, 4, 5, 6, 7, or 8. Examples of the cycloalkenyl group may include a 1-cyclopropen-1-yl group, a 2-cyclopropen-1-yl group, a 1-cyclobuten-1-yl group, a 2-cyclobuten-1-yl group, a 1-cyclopenten-1-yl group, a 2-cyclopenten-1-yl group, a 3-cyclopenten-1-yl group, a 1-cyclohexen-1-yl group, a 2-cyclohexen-1-yl group, a 3-cyclohexen-1-yl group, a 1-cyclohepten-1-yl group, a 2-cyclohepten-1-yl group, a 3-cyclohepten-1-yl group, a 4-cyclohepten-1-yl group, a 1-cycloocten-1-yl group, a 2-cycloocten-1-yl group, a 3-cycloocten-1-yl group, a 4-cycloocten-1-yl group, a 1,3-cyclopentadien-1-yl group, a 2,4-cyclopentadien-1-yl group, a 1,3-cyclohexadien-1-yl group, a 1,4-cyclohexadien-1-yl group, a 1,5-cyclohexadien-1-yl group, a 2,4-cyclohexadien-1-yl group, a 2,5-cyclohexadien-1-yl group, a 1,3-cycloheptadien-1-yl group, a 1,4-cycloheptadien-1-yl group, a 1,5-cycloheptadien-1-yl group, a 1,6-cycloheptadien-1-yl group, a 2,4-cycloheptadien-1-yl group, a 2,5-cycloheptadien-1-yl group, a 2,6-cycloheptadien-1-yl group, a 1,4-cycloheptadien-1-yl group, a 1,5-cycloheptadien-1-yl group, a 3,5-cycloheptadien-1-yl group, a 1,3-cyclooctadien-1-yl group, a 1,4-cyclooctadien-1-yl group, a 1,5-cyclooctadien-1-yl group, a 1,6-cyclooctadien-1-yl group, a 1,7-cyclooctadien-1-yl group, a 2,4-cyclooctadien-1-yl group, a 2,5-cyclooctadien-1-yl group, a 2,6-cyclooctadien-1-yl group, a 2,7-cyclooctadien-1-yl group, a 3,5-cyclooctadien-1-yl group, a 3,6-cyclooctadien-1-yl group, a 1,3,5-cycloheptatrien-1-yl group, a 1,3,6-cycloheptatrien-1-yl group, a 1,4,6-cycloheptatrien-1-yl group, a 2,4,6-cycloheptatrien-1-yl group, a 1,3,5-cyclooctatrien-1-yl group, a 1,3,6-cyclooctatrien-1-yl group, a 1,3,7-cyclooctatrien-1-yl group, a 1,4,6-cyclooctatrien-1-yl group, a 1,4,7-cyclooctatrien-1-yl group, a 1,5,7-cyclooctatrien-1-yl group, a 2,4,6-cyclooctatrien-1-yl group, a 2,4,7-cyclooctatrien-1-yl group, and a cyclooctatetraen-1-yl group.

As the alkynyl group, any of a linear, branched chain, or cyclic alkynyl group may be used. Further, the number of carbon atoms of the alkynyl group is preferably 2, 3, 4, 5, or 6. Examples of the alkynyl group may include an ethynyl group, a 1-propynyl group, a 2-propynyl group, a 1-butynyl group, a 2-butynyl group, a 3-butynyl group, a 1-methyl-2-propynyl group, a 1-pentynyl group, a 2-pentynyl group, a 3-pentynyl group, a 4-pentynyl group, a 1-methyl-2-butynyl group, a 1-methyl-3-butynyl group, a 2-methyl-3-butynyl group, a 3-methyl-1-butynyl group, a 1-ethyl-2-propynyl group, a 1,1-dimethyl-2-propynyl group, a 1-hexynyl group, a 2-hexynyl group, a 3-hexynyl group, a 4-hexynyl group, a 1-methyl-2-pentynyl group, a 1-methyl-3-pentynyl group, a 1-methyl-4-pentynyl group, a 2-methyl-3-pentynyl group, a 2-methyl-4-pentynyl group, a 3-methyl-1-pentynyl group, a 3-methyl-4-pentynyl group, a 4-methyl-1-pentynyl group, a 4-methyl-2-pentynyl group, a 1-butyl-2-propynyl group, a 1-ethyl-2-butynyl group, a 1-ethyl-3-butynyl group, a 2-ethyl-3-butynyl group, a 1,1-dimethyl-2-butynyl group, a 1,1-dimethyl-3-butynyl group, a 1,2-dimethyl-3-butynyl group, a 2,2-dimethyl-3-butynyl group, a 3,3-dimethyl-1-butynyl group, a 1-ethyl-1-methyl-2-propynyl group, a 1-(2-methylethyl)-2-propynyl group, a 2-cyclohexin-1-yl group, and a 3-cyclohexin-1-yl group.

As the aromatic group, a monocyclic or polycyclic aromatic ring may be used, and the aromatic ring may be condensed with a cycloalkane, a cycloalkene, an aromatic ring, or a hetero ring. The number of carbon atoms of the aromatic group is preferably 6, 7, 8, 9, 10, 11, 12, 13, or 14. Examples of the aromatic group include a phenyl group, a naphthyl group, and an anthracenyl group.

The heterocyclic group contains at least one or more hetero atoms such as a nitrogen atom, an oxygen atom or a sulfur atom as a ring-constituting atom, and those atoms may be condensed with a cycloalkane, a cycloalkene, an aromatic ring or a hetero ring, or form a spiro ring. The size of the hetero ring is preferably a 3-, 4-, 5-, 6-, 7- or 8-membered ring. Examples of the hetero ring may include an aziridinyl group, an azetidinyl group, a diazetidinyl group, a pyrrolidinyl group, a piperidino group, a homopiperidino group, a pyrazolidinyl group, an imidazolidinyl group, a triazolidinyl group, a tetrazolidinyl group, an oxazolidinyl group, an isoxazolidinyl group, a thiazolidinyl group, an isothiazolidinyl group, an oxadiazolidinyl group, a thiadiazolidinyl group, a piperazinyl group, a homopiperazinyl group, a triazepanyl group, a morpholino group, a thiomorpholino group, a quinuclidinyl group, a tropanyl group, a pyrrolinyl group, a pyrazolinyl group, an imidazolinyl group, an oxazolinyl group, a thiazolinyl group, an isoxazolinyl group, an isothiazolinyl group, a pyrrolyl group, an imidazolyl group, a pyrazolyl group, an oxazolyl group, a dihydrooxazolyl group, a tetrahydrooxazolyl group, an isoxazolyl group, a dihydroisoxazolyl group, a tetrahydroisoxazolyl group, a thiazolyl group, a dihydrothiazolyl group, a tetrahydrothiazolyl group, an isothiazolyl group, a dihydroisothiazolyl group, a tetrahydroisothiazolyl group, a triazolinyl group, a triazolyl group, an oxodiazolyl group, a dihydrooxodiazolyl group, a tetrahydrooxodiazolyl group, a thiadiazolyl group, a dihydrothiadiazolyl group, a tetrahydrothiadiazolyl group, a tetrazolinyl group, a tetrazolyl group, a furazanyl group, a dihydrofurazanyl group, a tetrahydrofurazanyl group, a piperideinyl group, a triazinanyl group, a pyridyl group, a dihydropyridyl group, a tetrahydropyridyl group, a pyrazinyl group, a dihydropyrazinyl group, a tetrahydropyrazinyl group, a pyrimidinyl group, a dihydropyrimidinyl group, a tetrahydropyrimidinyl group, a perhydropyrimidinyl group, a pyridazinyl group, a dihydropyridazinyl group, a tetrahydropyridazinyl group, a perhydropyridazinyl group, a triazinyl group, a dihydrotriazinyl group, a tetrahydrotriazinyl group, an oxazinyl group, a dihydrooxazinyl group, a tetrahydrooxazinyl group, an oxadiazinyl group, a dihydrooxadiazinyl group, a tetrahydrooxadiazinyl group, a thiazinyl group, a dihydrothiazinyl group, a tetrahydrothiazinyl group, a thiadiazinyl group, a dihydrothiadiazinyl group, a tetrahydrothiadiazinyl group, an azepinyl group, a dihydroazepinyl group, a tetrahydroazepinyl group, a perhydroazepinyl group, a diazepinyl group, a dihydrodiazepinyl group, a tetrahydrodiazepinyl group, a perhydrodiazepinyl group, an oxazepinyl group, a dihydrooxazepinyl group, a tetrahydrooxazepinyl group, a perhydrooxazepinyl group, an oxadiazepinyl group, a dihydrooxadiazepinyl group, a tetrahydrooxadiazepinyl group, a perhydrooxadiazepinyl group, a thiazepinyl group, a dihydrothiazepinyl group, a tetrahydrothiazepinyl group, a perhydrothiazepinyl group, a thiadiazepinyl group, a dihydrothiadiazepinyl group, a tetrahydrothiadiazepinyl group, a perhydrothiadiazepinyl group, a triazepinyl group, a dihydrotriazepinyl group, a tetrahydrotriazepinyl group, a perhydrotriazepinyl group, an azocinyl group, a dihydroazocinyl group, a tetrahydroazocinyl group, an oxohydroazocinyl group, a perhydroazocinyl group, a morphanyl group, a benzazocinyl group, an azepindolyl group, an indolinyl group, an indoleninyl group, an isoindolinyl group, an isoindoleninyl group, an indolyl group, a perhydroindolyl group, an isoindolyl group, a perhydroisoindolyl group, an indolizinyl group, an indolizidinyl group, an imidazopyridyl group, an indazolyl group, a dihydroindazolyl group, a perhydroindazolyl group, a benzimidazolyl group, a dihydrobenzimidazolyl group, a perhydrobenzimidazolyl group, a benzoxazolyl group, a dihydrobenzoxazolyl group, a perhydrobenzoxazolyl group, a benzothiazolyl group, a dihydrobenzothiazolyl group, a perhydrobenzothiazolyl group, a benzoxadiazolyl group, a benzothiadiazolyl group, a benzotriazolyl group, a purinyl group, a quinolinyl group, a dihydroquinolinyl group, a tetrahydroquinolinyl group, a perhydroquinolinyl group, a quinolizinyl group, a dihydroquinolizinyl group, a tetrahydroquinolizinyl group, an isoquinolinyl group, a dihydroisoquinolinyl group, a tetrahydroisoquinolinyl group, a perhydroisoquinolinyl group, a cinnolinyl group, a dihydrocinnolinyl group, a tetrahydrocinnolinyl group, a perhydrocinnolinyl group, a quinazolinyl group, a dihydroquinazolinyl group, a tetrahydroquinazolinyl group, a perhydroquinazolinyl group, a phthalazinyl group, a dihydrophthalazinyl group, a tetrahydrophthalazinyl group, a perhydrophthalazinyl group, a quinoxalinyl group, a dihydroquinoxalinyl group, a tetrahydroquinoxalinyl group, a perhydroquinoxalinyl group, a naphthyridinyl group, a dihydronaphthyridinyl group, a tetrahydronaphthyridinyl group, a perhydronaphthyridinyl group, a pteridinyl group, a quinolizidinyl group, a dihydrobenzoxazinyl group, a dihydrobenzothiazinyl group, a benzazepinyl group, a dihydrobenzazepinyl group, a tetrahydrobenzazepinyl group, a benzodiazepinyl group, a dihydrobenzodiazepinyl group, a tetrahydrobenzodiazepinyl group, a benzoxazepinyl group, a dihydrobenzoxazepinyl group, a tetrahydrobenzoxazepinyl group, a benzothiazepinyl group, a dihydrobenzothiazepinyl group, a tetrahydrobenzothiazepinyl group, a benzoxadiazepinyl group, a benzothiadiazepinyl group, a benzazepinyl group, a pyridoazepinyl group, a carbazolyl group, a dihydrocarbazolyl group, a tetrahydrocarbazolyl group, a perhydrocarbazolyl group, a β-carbolinyl group, a dihydro β-carbolinyl group, a tetrahydro β-carbolinyl group, a perhydro β-carbolinyl group, an acridinyl group, a dihydroacridinyl group, a tetrahydroacridinyl group, a perhydroacridinyl group, a phenazinyl group, a dihydrophenazinyl group, a tetrahydrophenazinyl group, a perhydrophenazinyl group, a phenothiazinyl group, a dihydrohydrophenothiazinyl group, a tetrahydrophenothiazinyl group, a perhydrophenothiazinyl group, a phenoxazinyl group, a dihydrophenoxazinyl group, a tetrahydrophenoxazinyl group, a perhydrophenoxazinyl group, a phenarsazinyl group, a phenanthridinyl group, a dihydrophenanthridinyl group, a tetrahydrophenanthridinyl group, a perhydrophenanthridinyl group, a phenanthrolinyl group, a dihydrophenanthrolinyl group, a tetrahydrophenanthrolinyl group, a perhydrophenanthrolinyl group, a perimidinyl group, a dihydroperimidinyl group, a tetrahydroperimidinyl group, a perhydroperimidinyl group, a pterinyl group, a pyrrolylidinyl group, a morphinanyl group, a hasubananyl group, a furyl group, a dihydrofuryl group, a tetrahydrofuryl group, a pyranyl group, a dihydropyranyl group, a tetrahydropyranyl group, an oxepinyl group, a dihydrooxepinyl group, a tetrahydrooxepinyl group, a perhydrooxepinyl group, a thienyl group, a dihydrothienyl group, a tetrahydrothienyl group, a thiopyranyl group, a dihydrothiopyranyl group, a tetrahydrothiopyranyl group, a thiepinyl group, a dihydrothiepinyl group, a tetrahydrothiepinyl group, a perhydrothiepinyl group, a benzofuryl group, a dihydrobenzofuryl group, a tetrahydrobenzofuryl group, a perhydrobenzofuryl group, an isobenzofuryl group, a dihydroisobenzofuryl group, a tetrahydroisobenzofuryl group, a perhydroisobenzofuryl group, a benzothienyl group, a dihydrobenzothienyl group, a tetrahydrobenzothienyl group, a perhydrobenzothienyl group, an isobenzothienyl group, a dihydroisobenzothienyl group, a tetrahydroisobenzothienyl group, a perhydroisobenzothienyl group, a benzopyranyl group, a dihydrobenzopyranyl group, a perhydrobenzopyranyl group, a benzothiopyranyl group, a dihydrobenzothiopyranyl group, a perhydrobenzothiopyranyl group, a benzoxepinyl group, a dihydrobenzoxepinyl group, a tetrahydrobenzoxepinyl group, a perhydrobenzoxepinyl group, a benzothiepinyl group, a dihydrobenzothiepinyl group, a tetrahydrobenzothiepinyl group, a perhydrobenzothiepinyl group, a benzofuryl group, a dihydrodibenzofuryl group, a tetrahydrodibenzofuryl group, a perhydrodibenzofuryl group, a xanthenyl group, a dihydroxanthenyl group, a tetrahydroxanthenyl group, a perhydroxanthenyl group, a benzothienyl group, a dihydrodibenzothienyl group, a tetrahydrodibenzothienyl group, a perhydrodibenzothienyl group, a thioxantenyl group, a dihydrothioxantenyl group, a tetrahydrothioxantenyl group, a perhydrothioxantenyl group, a phenoxathiinyl group, a dihydrophenoxathiinyl group, a tetrahydrophenoxathiinyl group, a perhydrophenoxathiinyl group, a dibenzodioxinyl group, a dihydrodibenzodioxinyl group, a tetrahydrodibenzodioxinyl group, a perhydrodibenzodioxinyl group, a thianthrenyl group, a dihydrothianthrenyl group, a tetrahydrothianthrenyl group, a perhydrothianthrenyl group, an oxiranyl group, an oxetanyl group, a thiiranyl group, a thietanyl group, an oxathiinyl group, a dihydrooxathiinyl group, a tetrahydrooxathiinyl group, a benzoxathiinyl group, a dihydrobenzoxathiinyl group, a tetrahydrobenzoxathiinyl group, a perhydrobenzoxathiinyl group, a benzodioxepanyl group, a dioxolanyl group, a dioxanyl group, a dithiolanyl group, a dithianyl group, a dioxoindanyl group, a benzodioxanyl group, a chromanyl group, a benzodithiolanyl group, and a benzodithianyl group, and in the case of an unsaturated heterocyclic group, a heterocyclic group in which at least a part is hydrogenated is also included.

Further, any two or three groups of the substituents R¹, R², R³, R⁴, R⁵, and R⁶ each may be combined together to form a ring. Examples of the ring include cyclopropane, cyclopropene, cyclobutane, cyclobutene, cyclopentane, cyclopentene, cyclopentadiene, cyclohexane, cyclohexene, cyclohexadiene, cycloheptane, cycloheptene, cycloheptadiene, cycloheptatriene, cyclooctane, cyclooctene, cyclooctadiene, cyclooctatriene, aziridine, azetidine, diazetidine, pyrrolidine, piperidine, homopiperidine, pyrazolidine, imidazolidine, triazolidine, tetrazolidine, oxazolidine, isoxazolidine, thiazolidine, isothiazolidine, oxazodiazolidine, thiadiazolidine, piperazine, and homopiperazine. Examples thereof include triazepane, morpholine, thiomorpholine, quinuclidine, tropane, pyrroline, pyrazoline, imidazoline, oxazoline, thiazoline, isoxazoline, isothiazoline, dihydrooxazole, tetrahydrooxazole, dihydroisoxazole, tetrahydroisoxazole, dihydrothiazole, tetrahydrothiazole, dihydroisothiazole, tetrahydroisothiazole, triazoline, dihydrooxadiazole, tetrahydrooxadiazole, dihydrothiadiazole, tetrahydrothiadiazole, dihydrofurazan, tetrahydrofurazan, piperidyne, triazinane, dihydropyridine, tetrahydropyridine, dihydropyrazine, tetrahydropyrazine, dihydropyrimidine, tetrahydropyrimidine, perhydropyrimidine, dihydropyridazine, tetrahydropyridazine, perhydropyridazine, oxazine, dihydro oxadiazine, tetrahydro oxazine, oxadiazine, dihydro oxadiazine, tetrahydro oxadiazine, thiazine, dihydrothiazine, tetrahydrothiazine, thiadiazine, dihydrothiadiazine, tetrahydrothiadiazine, dihydroazepine, tetrahydroazepine, perhydroazepine, dihydrodiazepine, tetrahydrodiazepine, perhydrodiazepine, oxazepine, dihydrooxazepine, tetrahydrooxazepine, perhydrooxazepine, oxadiazepine, dihydrooxadiazepine, tetrahydrooxadiazepine, perhydrooxadiazepine, thiazepine, dihydrothiazepine, tetrahydrothiazepine, perhydrothiazepine, thiadiazepine, dihydrothiadiazepine, tetrahydrothiadiazepine, perhydrothiadiazepine, triazepine, dihydrotriazepine, tetrahydrotriazepine, perhydrotriazepine, azocine, dihydroazocine, tetrahydroazocine, oxohydroazocine, perhydroazocine, morphan, azepindole, indoline, indolenine, isoindoline, isoindolenine, perhydroindole, perhydroisoindole, perhydroisoindole, indolizidine, dihydroindazole, perhydroindazole, dihydrobenzimidazole, perhydrobenzimidazole, dihydrobenzoxazole, perhydrobenzoxazole, dihydrobenzothiazole, perhydrobenzothiazole, dihydroquinoline, tetrahydroquinoline, perhydroquinoline, quinolizine, dihydroquinolizine, tetrahydroquinolizine, dihydroisoquinoline, tetrahydroisoquinoline, perhydroisoquinoline, dihydrocinnoline, tetrahydrocinnoline, perhydrocinnoline, dihydroquinazoline, tetrahydroquinazoline, perhydroquinazoline, dihydrophthalazine, tetrahydrophthalazine, perhydrophthalazine, dihydroquinoxaline, tetrahydroquinoxaline, perhydroquinoxaline, dihydronaphthyridine, tetrahydronaphthyridine, perhydronaphthyridine, quinolizidine, dihydrobenzoxazine, dihydrobenzothiazine, dihydrobenzazepine, tetrahydrobenzazepine, perhydrobenzazepine, dihydrobenzodiazepine, tetrahydrobenzodiazepine, perhydrobenzodiazepine, dihydrobenzoxazepine, tetrahydrobenzoxazepine, perhydrobenzoxazepine, dihydrobenzothiazepine, tetrahydrobenzothiazepine, perhydrobenzothiazepine, dihydrocarbazole, tetrahydrocarbazole, perhydrocarbazole, dihydro β-carboline, tetrahydro β-carboline, perhydro β-carboline, dihydroacridine, tetrahydroacridine, perhydroacridine, dihydrophenazine, tetrahydrophenazine, perhydrophenazine, dihydrohydrophenothiazine, tetrahydrophenothiazine, perhydrophenothiazine, dihydrophenoxazine, tetrahydrophenoxazine, perhydrophenoxazine, dihydrophenanthridine, tetrahydrophenanthridine, perhydrophenanthridine, dihydrophenanthroline, tetrahydrophenanthroline, perhydrophenanthroline, dihydroperimidine, tetrahydroperimidine, perhydroperimidine, pyrrolidine, morphinan, hasubanan, dihydrofuran, tetrahydrofuran, pyran, dihydropyran, tetrahydropyran, dihydrooxepin, tetrahydrooxepin, perhydrooxepin, dihydrothiophene, tetrahydrothiophene, thiopyran, dihydrothiopyran, tetrahydrothiopyran, dihydrothiepine, tetrahydrothiepine, perhydrothiepine, dihydrobenzofuran, tetrahydrobenzofuran, perhydrobenzofuran, dihydroisobenzofuran, tetrahydroisobenzofuran, perhydroisobenzofuran, dihydrobenzothiophene, tetrahydrobenzothiophene, perhydrobenzothiophene, dihydroisobenzothiophene, tetrahydrobenzothiophene, perhydrobenzothiophene, benzopyran, dihydrobenzopyran, perhydrobenzopyran, benzothiopyran, dihydrobenzothiopyran, perhydrobenzothiopyran, dihydrobenzoxepin, tetrahydrobenzoxepin, perhydrobenzoxepin, dihydrobenzothiepine, tetrahydrobenzothiepin, perhydrobenzothiepine, dihydrodibenzofuran, tetrahydrodibenzofuran, perhydrodibenzofuran, xanthene, dihydroxanthene, tetrahydroxanthene, perhydroxanthene, dihydrodibenzothiophene, tetrahydrodibenzothiophene, perhydrodibenzothiophene, thioxanthene, dihydrothioxanthene, tetrahydrothioxanthene, perhydrothioxanthene, dihydrophenoxathiin, tetrahydrophenoxathiin, perhydrophenoxathiin, dihydrodibenzodioxin, tetrahydrodibenzodioxin, perhydrodibenzodioxin, dihydrothianthrene, tetrahydrothianthrene, perhydrothianthrene, oxirane, oxetane, thiirane, thietane, dihydrooxathiin, tetrahydrooxathiin, dihydrobenzoxathiin, tetrahydrobenzoxathiin, perhydrobenzoxathiin, benzodioxepane, dioxolan, dioxane, dithiolane, dithiane, dioxoindane, benzodioxane, chromane, benzodithiolane, benzodithiane, norbornane, 1-azanorbornane, 2-azanorbornane, 7-azanorbornane, norbornene, 1-azanorbornene, 2-azanorbornene, 7-azanorbornene, norbornadiene, bicyclo[2,2,2]octane, 1-azabicyclo[2,2,2]octane, 2-azabicyclo[2,2,2]octane, norpinane, norpinene, norcarane, and norcanene, and in the case of an unsaturated ring, a ring in which at least a part is hydrogenated is also included. Further, in the case of forming a ring, it is preferable that any two or three substituents of R³, R⁴, R⁵, and R⁶ form a ring.

Further, examples of the substituent which the alkyl group, the cycloalkyl group, the alkenyl group, the cycloalkenyl group, the alkynyl group, the aromatic group, and the heterocyclic group may include groups selected from a hydroxyl group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclyl thio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an Rx(Ry)N group, and an Rx(Ry)(Rz)N⁺ group. Herein, Rx, Ry, and Rz each independently represent a hydrogen atom, an alkyl group, a cycloalkyl group, an alkenyl group, a cycloalkenyl group, an alkynyl group, an aromatic hydrocarbon group, or a heterocyclic group. Further, two or more of Rx, Ry, and Rz may be bonded to each other to form a saturated or unsaturated hetero ring, and this ring can also form a condensed ring or a spiro ring with an aliphatic ring or a hetero ring and can also form a condensed ring with an aromatic ring.

Incidentally, Rx, Ry and Rz excluding the case of a hydrogen atom and the alkyl group, the cycloalkyl group, the alkenyl group, the cycloalkenyl group, the alkynyl group, the aromatic group, and the heterocyclic group as the substituent which are described herein include the same groups as the groups represented by R¹, R², R³, R⁴, R⁵, and R⁶. Further, the alkyl group of the alkoxy group and the alkylthio group as substituents has the same definition as the definition of the alkyl group in R¹, R², R³, R⁴, R⁵, and R⁶ described above, and the aryl group of the aryloxy group and the arylthio group has the same definition as the definition of the aromatic group in R¹, R², R³, R⁴, R⁵, and R⁶ described above.

Further, examples of a guanidino group, an acyl group, a substituted sulfonyl group, a heterocyclyloxy group, a heterocyclyl thio group, a carbamoyl group, a ureido group, an amide group, a sulfamoyl group, an acyloxy group, a sulfonamide group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, and an alkoxysulfonyl group as substituents are as follows.

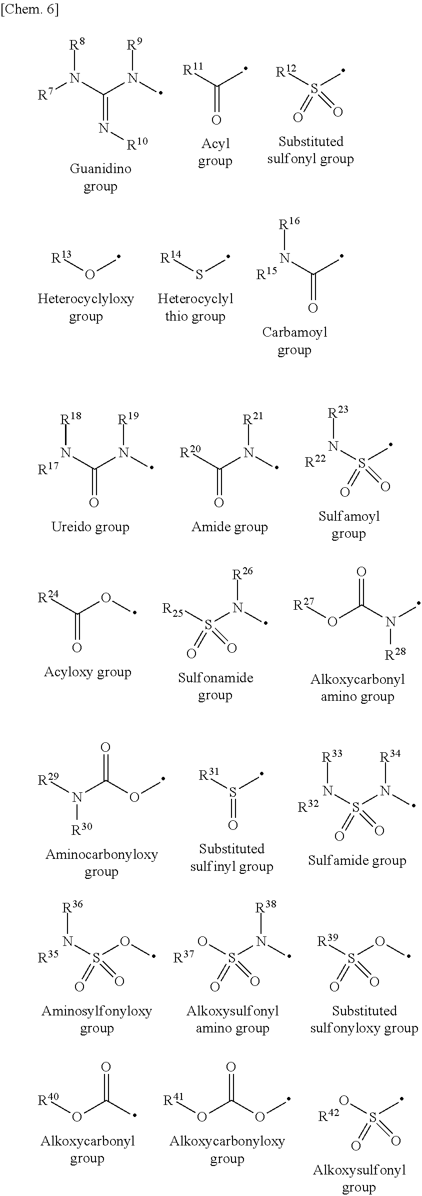

(In the exemplary groups, R⁷, R⁸, R⁸, R¹⁰, R¹¹, and R¹²; R¹⁵, R¹⁶, R¹⁷, R¹⁷, R¹⁹, R²⁰, R²¹, R²², R²³, R²⁴, R²⁶, R²⁸, R²⁹, R³⁰, R³¹, R³², R³³, R³⁴, R³⁵, and R³⁶; and R³⁸ and R³⁹ represent a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group. R²⁵, R²⁷, R³⁷, R⁴⁰, R⁴¹, and R⁴² represent a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group. R¹³ and R¹⁴ represent a substituted or unsubstituted heterocyclic group. Further, examples of substituents of those substituted alkyl group, substituted cycloalkyl group, substituted alkenyl group, substituted cycloalkenyl group, substituted alkynyl group, substituted aromatic group, and substituted heterocyclic group include the same substituents as substituents of those groups in R¹, R², R³, R⁴, R⁵, and R⁶ described above.)

The groups represented by R¹, R², R³, R⁴, R⁵, and R⁶ are preferably each independently a hydrogen atom, a substituted or unsubstituted linear or branched chain alkyl group having carbon number of 1 to 6, a substituted or unsubstituted cycloalkyl group having carbon number of 3 to 8, a substituted or unsubstituted linear or branched alkenyl group having carbon number of 2 to 6, a substituted or unsubstituted cycloalkenyl group having carbon number of 3 to 8, a substituted or unsubstituted linear or branched alkynyl group having carbon number of 2 to 6, a substituted or unsubstituted monocyclic or polycyclic aromatic group having carbon number of 6 to 14, or a substituted or unsubstituted 3- to 8-membered heterocyclic group containing at least one of a nitrogen atom, an oxygen atom, or a sulfur atom as a ring-constituting atom. The groups represented by R¹, R², R³, R⁴, R⁵, and R⁶ each independently is a hydrogen atom or an alkyl group having carbon number of 1 to 6, or two of R³, R⁴, R⁵, and R⁶ are coupled to form a cycloalkyl group having carbon number of 3 to 8 is preferable in terms of ease of availability of a raw material. Particularly, it is preferable that both of R¹ and R² represent a hydrogen atom or one of R¹ and R² represents a methyl group.

In the primary or secondary amine compound-acidic polysaccharide conjugate represented by Formula (I) or (II), and the amine form that is an important intermediate represented by Formula (III) or (XII), D is a structure that represents a residue excluding a hydrogen atom in the primary or secondary amino group of the primary or secondary amine compound DH, and DH specifically represents a compound represented by the following Formula (XIII).

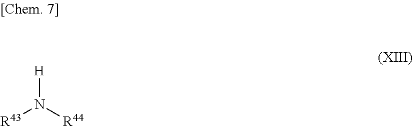

R⁴³ and R⁴⁴ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group or a substituted or unsubstituted heterocyclic group, an R⁴⁵O-group, an R⁴⁶S-group, or an R⁴⁷(R⁴⁸)N-group in which R⁴⁵, R⁴⁶, R⁴⁷, and R⁴⁸ are each independently a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, a substituted or unsubstituted heterocyclic group, or an Rx(Ry)N group. Here, R⁴³ and R⁴⁴ do not become hydrogen atoms at the same time. Further, two of R⁴³ and R⁴⁴ may form a single or double bond so as to form a saturated or unsaturated hetero ring, the ring can also form a condensed ring or a spiro ring with an aliphatic ring or a hetero ring, and a condensed ring can be formed with an aromatic ring. Here, the alkyl group, the cycloalkyl group, the alkenyl group, the cycloalkenyl group, the alkynyl group, the aromatic group, or the heterocyclic group described herein has the same meaning as definition in R¹, R², R³, R⁴, R⁵, and R⁶ described above. Further, Rx and Ry described herein have the same meaning as definitions of Rx and Ry in the Rx(Ry)N group that is the substituent of R¹, R², R³, R⁴, R⁵, and R⁶ described above.

Examples of the saturated or unsaturated hetero ring formed by R⁴³ and R⁴⁴ being bonded to each other may include aziridine, azetidine, diazetidine, pyrrolidine, piperidine, homopiperidine, pyrazolidine, imidazolidine, triazolidine, tetrazolidine, oxazolidine, isoxazolidine, thiazolidine, isothiazolidine, oxadiazolidine, thiadiazolidine, piperazine, homopiperazine, triazepane, morpholine, thiomorpholine, pyrroline, pyrazoline, imidazoline, triazoline, tetrazoline, dihydrofurazan, tetrahydrofurazan, piperideine, triazinane, dihydropyridine, dihydropyrazine, tetrahydropyrazine, dihydropyrimidine, tetrahydropyrimidine, perhydropyrimidine, dihydropyridazine, tetrahydropyridazine, perhydropyridazine, dihydrotriazine, tetrahydrotriazine, tetrahydro oxazine, dihydro oxadiazine, tetrahydro oxadiazine, tetrahydrothiazine, dihydrothiadiazine, tetrahydrothiadiazine, azepine, dihydroazepine, tetrahydroazepine, diazepine, dihydrodiazepine, tetrahydrodiazepine, perhydrodiazepine, oxazepine, dihydrooxazepine, tetrahydrooxazepine, perhydrooxazepine, oxadiazepine, dihydrooxadiazepine, tetrahydrooxadiazepine, perhydrooxadiazepine, thiazepine, dihydrothiazepine, tetrahydrothiazepine, perhydrothiazepine, thiadiazepine, dihydrothiadiazepine, tetrahydrothiadiazepine, perhydrothiadiazepine, triazoline, dihydrotriazepine, tetrahydrotriazepine, perhydrotriazepine, dihydroazocine, tetrahydroazocine, oxohydroazocine, perhydroazocine, morphan, azepindole, indoline, isoindoline, dihydroindazole, perhydroindazole, dihydrobenzimidazole, perhydrobenzimidazole, dihydrobenzoxazole, perhydrobenzoxazole, dihydrobenzothiazole, perhydrobenzothiazole, dihydroquinoline, tetrahydroquinoline, perhydroquinoline, dihydroisoquinoline, tetrahydroisoquinoline, perhydroisoquinoline, dihydrocinnoline, tetrahydrocinnoline, perhydrocinnoline, dihydroquinazoline, tetrahydroquinazoline, perhydroquinazoline, dihydrophthalazine, tetrahydrophthalazine, perhydrophthalazine, dihydroquinoxaline, tetrahydroquinoxaline, perhydroquinoxaline, dihydronaphthyridine, tetrahydronaphthyridine, perhydronaphthyridine, benzazepine, dihydrobenzazepine, tetrahydrobenzazepine, benzodiazepine, dihydrobenzodiazepine, tetrahydrobenzodiazepine, dihydrobenzoxazepine, tetrahydrobenzoxazepine, dihydrobenzothiazepine, tetrahydrobenzothiazepine, benzazepine, carbazole, dihydrocarbazole, tetrahydrocarbazole, perhydrocarbazole, β-carboline, dihydro β-carboline, tetrahydro β-carboline, perhydro β-carboline, dihydroacridine, dihydrophenazine, phenothiazine, phenoxazine, phenarsazine, dihydrophenanthridine, dihydroperimidine, pterin, morphinan, and hasubanan, and in the case of an unsaturated hetero ring, a ring in which at least a part is hydrogenated is also included. Further, a structure in which two or more of those structures are bonded to each other directly or via an alkylene group can also be employed, and the heterocyclic group has the same definition as that of the heterocyclic group represented by R¹, R², R³, R⁴, R⁵, and R⁶ described above and can have a substituent.

Examples of a substituent which the alkyl group, the cycloalkyl group, the alkenyl group, the cycloalkenyl group, the alkynyl group, the aromatic group, the heterocyclic group, the R⁴⁵O— group, the R⁴⁶S— group, the R⁴⁷(R⁴⁸)N— group and the saturated or unsaturated hetero ring formed by R⁴³ and R⁴⁴ being bonded to each other may have include the same substituents of those groups in R¹, R², R³, R⁴, R₅, and R⁶ described above.

D is a structure that represents a residue excluding a hydrogen atom bonded to a nitrogen atom used for bonding to a linker in the primary or secondary amine compound DH. The primary or secondary amine compound DH is preferably a compound having bioactivity. Examples of the compound having bioactivity may include active ingredients such as a medical drug, a quasi-drug, a medical device, an in-vitro diagnostic medical drug, a regenerative medical product, a medical drug for animals, an agricultural chemical, and a supplement. If the released primary or secondary amine compound DH has bioactivity and can form a urethane bond by a primary or secondary amino group of the compound, the structure of the compound is not limited, and a known compound which can be used as a compound can be used.

The amine form represented by Formula (III) or (XI) may form a salt with an inorganic acid or an organic acid. Examples of the inorganic acid include hydrochloric acid, sulfuric acid, and nitric acid. Examples of the organic acid include trifluoroacetic acid, methanesulfonic acid, toluenesulfonic acid, benzenesulfonic acid, and trifluoromethanesulfonic acid. Regarding a salt formed with an inorganic acid or an organic acid, preferably, an amino group existing at the molecule terminal of the amine form represented by Formula (III) or (XII) forms a salt with an inorganic acid or an organic acid.

The polymer providing a structure derived from the acidic polysaccharide has at least one carboxy group in a monomer unit and can be represented by Formula (IV):

Poly-CO₂H (IV)

Hereinafter, the moiety of Poly is also referred to as an “acidic polysaccharide residue”. The acidic polysaccharide has a plurality of carboxy groups in one molecule. In the conjugate of the present invention, a plurality of amine forms represented by Formula (III) or (XII) may be condensed.

For example, a conjugate according to an aspect of the present invention can be represented by the following Formula (XX), which is equivalent to the compound represented by Formula (I):

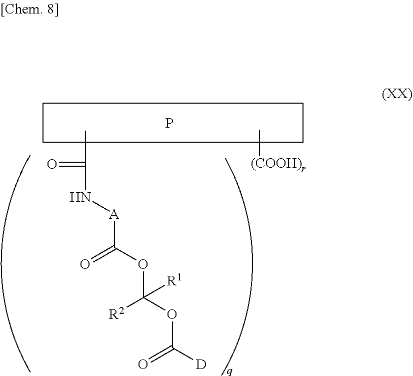

in Formula (I), D, R¹, R², and A are as defined above,

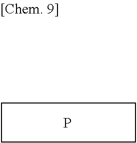

a moiety represented by P represents a polymer (polymer chain) excluding a group derived from the carboxy group (a group represented by —C(═O)NH-A-C(═O)OC(—R¹)(—R²)OC(═O)D) and the carboxy group of the acidic polysaccharide, q represents a number of a compound (an amine form represented by Formula (LII)) condensed with the polymer (i.e., a number of a group derived from the carboxy group), and r represents a number of the carboxy group.

Incidentally, the Formula (XX) does not only mean that q of the groups represented by —C(═O)NH-A-C(═O)OC(—R¹)(—R²)OC(═O)D) and r of the groups represented by —COOH are continuously arranged in blocks in the acidic polysaccharide. It should be understood that the group represented by —C(═O)NH-A-C(═O)OC(—R¹)(—R²)O(═O)D and the group represented by —COOH may be randomly arranged in the polymer chain, or may be arranged in blocks or in an alternating regular manner.

Further, a conjugate according to another aspect of the present invention can be represented by the following Formula (XXX), which is equivalent to the compound represented by Formula (II):

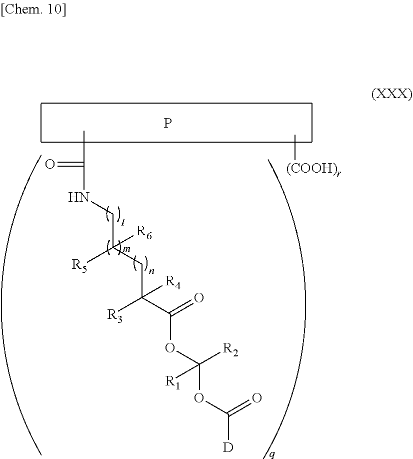

in Formula (XXX), D, R¹, R², R⁴, R⁵, R⁶, r, and P are as defined above, and q represents a number of a compound (an amine form represented by Formula (XII)) condensed with the polymer.

The values of q and r are determined according to the ratio of the compound (the amine form represented by Formula (III) or (XII)) which is condensed with the acidic polysaccharide. The degree to which the amine form represented by Formula (III) or (XII) is condensed with one molecular chain of the acidic polysaccharide can be appropriately changed and adjusted according to the structure represented by DH, the type of the acidic polysaccharide, and the like. The degree to which the compound having the structure represented by DH is introduced can be indicated as “introduction ratio” in the present specification. The introduction ratio can be determined by a method such as calculation of integration ratio by ¹H NMR or calculation of concentration by spectroscopy. Examples of the spectroscopy include UV-visible absorption spectroscopy. In the present specification, the introduction ratio (mol %) by the calculation of molar ratio, the introduction ratio (wt %) by the calculation of concentration, and the like can be collectively referred to as simply “introduction ratio”, and is represented by q/(q+r)×100. The introduction ratio based on the calculation of molar ratio (calculation of integration ratio by ¹H NMR) is not particularly limited, and may be, for example, in a range of 1 to 80 mol %. Here, q and r are preferably values that fall within the above-described range of the introduction ratio. In a case where the conjugate of the present invention is converted to an aqueous solution, the introduction ratio is preferably in a range of 2 to 70%, preferably in a range of 5 to 60%, and more preferably in a range of 10 to 50% from the viewpoint of having high filterability at a high drug concentration (the concentration of the primary or secondary amine compound DH).

In the acidic polysaccharide, the carboxy group remaining without being condensed with the amine form represented by Formula (III) or (XII) may exist as a free carboxy group, a salt may be formed using a metal such as lithium, sodium, potassium, magnesium, or calcium or an organic base such as triethylamine, tributylamine, and pyridine, or a salt may be formed using tetrabutylammonium hydroxide.

The conjugate of the present invention preferably has high filterability when converted to an aqueous solution. Specifically, when the concentration of the primary or secondary amine compound DH in the aqueous solution is 0.5 (w/w) %, the filter filtration rate is preferably 50 wt % or more, more preferably 80 wt % or more, and particularly preferably 90 wt % or more.

This filter filtration rate (wt %) can be measured by the method described in Examples below. Specifically, an aqueous solution containing 70 to 90 mg of the conjugate of the present invention is added to a polyvinylidene fluoride (PVDF) centrifugal filter unit having a pore size of 0.22 μm and centrifuged at 25° C. and 12000 G for 90 minutes. Thereafter, the liquid passing through the filter is weighed, whereby the filter filtration rate can be calculated from the weight ratio of the liquid passing through the filter to the aqueous solution added to the filter unit.

Examples of the acidic polysaccharide include naturally occurring polysaccharides such as alginic acid, hyaluronic acid, heparin, chondroitin, chondroitin sulfate (A, B, C, D, and E), keratan sulfate, heparan sulfate, dermatan sulfate, pectin (homogalacturonan and rhamnogalacturonan), xanthan gum, xylan, and sacran; carboxymethyl cellulose, carboxymethyl chitin, carboxymethyl chitosan, carboxymethyl dextran, carboxymethyl amylose, and succinyl chitosan. Examples of glycosaminoglycan include hyaluronic acid, heparin, chondroitin, chondroitin sulfate (A, B, C, D, and E), keratan sulfate, heparan sulfate, and dermatan sulfate.

These acidic polysaccharides may be cross-linked or chemically modified by various methods in advance, and may be further cross-linked or chemically modified after forming the primary or secondary amine compound-acidic polysaccharide conjugate in some cases. Further, these acidic polysaccharides may form a pharmaceutically acceptable salt, for example, a salt with a metal such as lithium, sodium, potassium, magnesium, or calcium, or an organic base such as triethylamine, tributylamine and pyridine, or a salt may be formed using tetrabutylammonium hydroxide.

Any molecular weight of the acidic polysaccharide can be employed without particular limitation as long as the molecular weight is a molecular weight of a generally used product. The molecular weight can also be appropriately adjusted according to the use of the conjugate and the properties required of the conjugate. Examples of a preferred molecular weight include 10 kDa or more, however it is not limited thereto.

Poly means a partial structure of the acidic polysaccharide represented by the Formula (IV) excluding a carboxy group moiety used for condensation with the amine form represented by the Formula (III) or (XII). As the Poly, an acidic polysaccharide residue, a glycosaminoglycan residue, a chondroitin residue, a chondroitin sulfate residue, and a hyaluronic acid residue can be exemplified as a preferable aspect. These residues each mean a partial structure of acidic polysaccharide, glycosaminoglycan, chondroitin, chondroitin sulfate, and hyaluronic acid excluding a carboxy group condensed with a compound (III) or (XII).

In the case of using the acidic polysaccharide as a carrier of the conjugate, a pharmaceutical composition having a high drug concentration can be prepared even if the molecular weight of the polymer is increased as shown in the test results described below. For example, in a case where the conjugate of the present invention is converted to an aqueous solution, the concentration of the primary or secondary amine compound DH in the aqueous solution is preferably in a range of 0.3 to 30 (w/w) %, and more preferably in a range of 0.5 to 20 (w/w) %.

As described above, the novel primary or secondary amine compound-acidic polysaccharide conjugate of the present invention can be expected to have higher safety than other polymers, and is an excellent conjugate that enables the introduction of a drug in an amount required for the drug effect onset without being significantly affected by the polymer molecular weight.

A production example of the primary or secondary amine compound-acidic polysaccharide conjugate represented by Formula (I) is as follows:

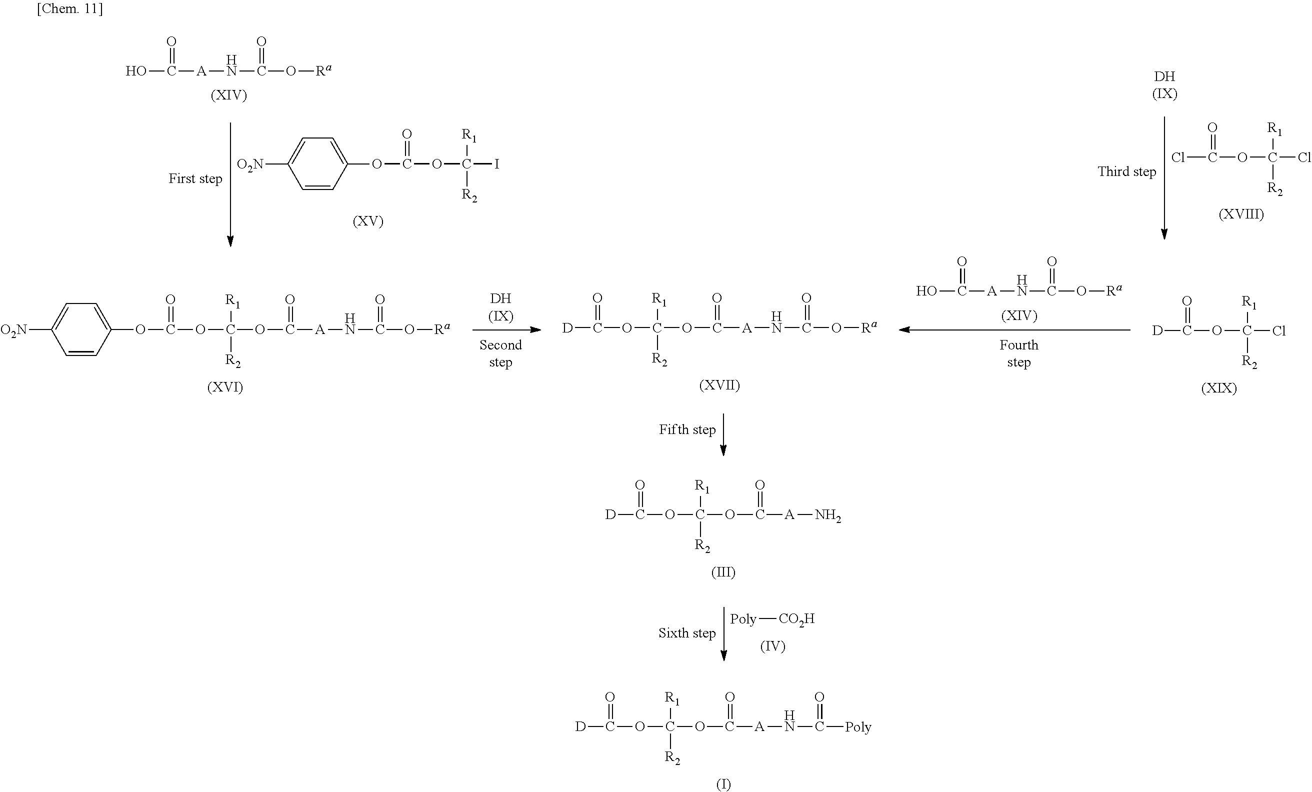

(in the formula, Ra represents a benzyl group or a t-butyl group, and R¹, R², D, A, and Poly are as defined above.)

First Step

This step is to produce the p-nitrophenyl ester form represented by the Formula (XVI) by reacting the protected amino acid represented by the Formula (XIV) with the iodomethyl carbonate form represented by the Formula (XV). This step is performed by reacting a carboxylic acid of the protected amino acid represented by the Formula (XIV) with a metal oxide or a base to form a carboxylate salt, and then reacting with the iodomethyl carbonate form represented by the Formula (XV). Alternatively, this step is performed by reacting the protected amino acid represented by the Formula (XIV) with the iodomethyl carbonate form represented by the Formula (XVI) in the presence of a base.

Upon forming the salt, the step is preferably performed in a solvent. As the organic solvent, for example, methanol, ethanol, methylene chloride, chloroform, dichloroethane, ethyl acetate, acetone, benzene, toluene, xylene, acetonitrile, tetrahydrofuran, dioxane, diethyl ether, diisopropyl ether, dimethoxyethane, or the like can be used. As the metal oxide, for example, silver oxide (I) and mercury oxide (II) can be used. As the base, cesium carbonate, sodium hydrogen carbonate, sodium carbonate, potassium bicarbonate, potassium carbonate, sodium hydroxide, potassium hydroxide, lithium hydroxide, or the like can be used. As the reaction temperature, the step can proceed usually in a range of −30° C. to 200° C. and preferably in a range of −15° C. to 80° C.

Upon performing the esterification, the step is preferably performed in a solvent, for example, an organic solvent medium such as dimethylformamide, methylene chloride, chloroform, dichloroethane, ethyl acetate, acetone, benzene, toluene, xylene, acetonitrile, tetrahydrofuran, dioxane, dimethylsulfoxide, diethyl ether, diisopropyl ether, or dimethoxyethane can be used. As the base to coexist, cesium carbonate, sodium hydrogen carbonate, sodium carbonate, potassium bicarbonate, potassium carbonate, sodium hydroxide, potassium hydroxide, lithium hydroxide, pyridine, N,N-diisopropylethylamine, triethylamine, 2,6-lutidine, 4-dimethylaminopyridine, diazabicycloundecene, 1,8-bis(dimethylamino)naphthalene, metal bis(trimethylsilyl)amide, lithium diisopropylamide, or the like can be used. As the reaction temperature, the step can proceed usually in a range of −30° C. to 200° C. and preferably in a range of 0° C. to 80° C.

Second Step

This step is to produce the ester form represented by the Formula (XVII) by reacting the primary or secondary amine compound represented by the Formula (IX) with the p-nitrophenyl ester form represented by the Formula (XVI). In this step, for example, in a case where the primary or secondary amine compound represented by the Formula (IX) forms a salt with an organic acid or an inorganic acid, a base can be added, if necessary. Upon performing this step, this step is preferably performed in a solvent, for example, an organic solvent such as methylene chloride, chloroform, dichloroethane, ethyl acetate, acetone, benzene, toluene, xylene, dimethylformamide, acetonitrile, tetrahydrofuran, dioxane, diethyl ether, diisopropyl ether, or dimethoxyethane can be used, and if necessary, a two-phase system or a mixed solvent can be formed by adding water. As the base, for example, pyridine, N,N-diisopropylethylamine, triethylamine, 2,6-lutidine, 4-dimethylaminopyridine, diazabicycloundecene, 1,8-bis(dimethylamino)naphthalene, metal bis(trimethylsilyl)amide, lithium diisopropylamide, or the like can be used. As the reaction temperature, the step can proceed usually in a range of −78° C. to 200° C. and preferably in a range of −20° C. to 80° C.

Third Step

This step is to produce the chloroalkyl ester form represented by the Formula (XIX) from the primary or secondary amine compound represented by the Formula (IX). This step is performed by reacting the primary or secondary amine compound represented by the Formula (IX) with the chloroalkyl chloroformate represented by the Formula (XVIII) in the presence of a base.

Upon performing this step, this step is preferably performed in a solvent, for example, an organic solvent such as methylene chloride, chloroform, dichloroethane, ethyl acetate, acetone, benzene, toluene, xylene, dimethylformamide, acetonitrile, tetrahydrofuran, dioxane, diethyl ether, diisopropyl ether, or dimethoxyethane can be used, and if necessary, a mixed solvent of an organic solvent and water can be used. As the base, for example, sodium hydrogen carbonate, sodium carbonate, potassium bicarbonate, potassium carbonate, sodium hydroxide, potassium hydroxide, lithium hydroxide, pyridine, N,N-diisopropylethylamine, triethylamine, 2,6-lutidine, 4-dimethylaminopyridine, diazabicycloundecene, 1,8-bis(dimethylamino)naphthalene, metal bis(trimethylsilyl)amide, lithium diisopropylamide, or the like can be used. As the reaction temperature, the step can proceed usually in a range of −78° C. to 200° C. and preferably in a range of −20° C. to 80° C.

Fourth Step

This step is to produce an ester form represented by the Formula (XVII) by reacting the chloroalkyl ester form represented by the Formula (XIX) with the protected amino acid represented by the Formula (XIV). This step is performed by reacting a carboxylic acid of the protected amino acid represented by the Formula (XIV) with a base to form a carboxylate salt, and then reacting with the chloroalkyl ester form represented by the Formula (XIX). Alternatively, this step is performed by reacting the protected amino acid represented by the Formula (XIV) with the chloroalkyl ester form represented by the Formula (XIX) in the presence of a base.

Upon forming the salt, the step is preferably performed in a solvent. As the organic solvent, for example, methanol, ethanol, methylene chloride, chloroform, dichloroethane, ethyl acetate, acetone, benzene, toluene, xylene, acetonitrile, tetrahydrofuran, dioxane, diethyl ether, diisopropyl ether, dimethoxyethane, or the like can be used. Further, as the base, for example, cesium carbonate, sodium hydrogen carbonate, sodium carbonate, potassium bicarbonate, potassium carbonate, sodium hydroxide, potassium hydroxide, lithium hydroxide, and the like can be used. As the reaction temperature, the step can proceed usually in a range of −30° C. to 200° C. and preferably in a range of −15° C. to 80° C.

Upon performing the esterification, the step is preferably performed in a solvent, for example, an organic solvent such as dimethylformamide, methylene chloride, chloroform, dichloroethane, ethyl acetate, acetone, benzene, toluene, xylene, acetonitrile, tetrahydrofuran, dioxane, dimethylsulfoxide, diethyl ether, diisopropyl ether, or dimethoxyethane can be used. As the base to coexist, cesium carbonate, sodium hydrogen carbonate, sodium carbonate, potassium bicarbonate, potassium carbonate, sodium hydroxide, potassium hydroxide, lithium hydroxide, pyridine, N,N-diisopropylethylamine, triethylamine, 2,6-lutidine, 4-dimethylaminopyridine, diazabicycloundecene, 1,8-bis(dimethylamino)naphthalene, metal bis(trimethylsilyl)amide, lithium diisopropylamide, or the like can be used. As the reaction temperature, the step can proceed usually in a range of −30° C. to 200° C. and preferably in a range of 0° C. to 80° C.

Fifth Step

This step is to produce the amine form represented by the Formula (III) by deprotecting the ester form represented by the Formula (XVII).

In this step, in a case where Ra represents a benzyl group, the ester form is deprotected by catalytic hydrogen addition so that the amine form represented by Formula (III) can be produced. For example, a platinum catalyst such as platinum oxide or platinum carbon, a palladium catalyst such as palladium carbon, palladium black, or palladium oxide, or a nickel catalyst such as Raney nickel can be used. Upon performing this step, this step is preferably performed in a solvent, and for example, methanol, ethanol, isopropyl alcohol, tetrahydrofuran, dimethylformamide, dioxane, water, or the like can be used. As the reaction temperature, the step can proceed usually in a range of −50° C. to 200° C. and preferably in a range of 10° C. to 100° C.

In this step, in a case where Ra represents a t-butyl group, the amine form represented by Formula (III) can be produced by deprotection using an acid. As the acid, for example, hydrogen chloride, hydrochloric acid, sulfuric acid, nitric acid, methanesulfonic acid, benzenesulfonic acid, p-toluenesulfonic acid, trifluoromethanesulfonic acid, trifluoroacetic acid, or the like can be used. The amine form represented by Formula (III) which is obtained in this step is produced by forming salts with those acids. In this step, the reaction can proceed in the absence of a solvent or in a solvent, and as the solvent, for example, ethyl acetate, dioxane, methanol, ethanol, 1-propanol, 2-propanol, diethyl ether, water, or the like can be used. As the reaction temperature, the step can proceed usually in a range of −50° C. to 200° C. and preferably in a range of 0° C. to 80° C.

Sixth Step

This step is to produce the primary or secondary amine compound-acidic polysaccharide conjugate represented by the Formula (I) by condensing the amine form represented by the Formula (III) with the acidic polysaccharide represented by the Formula (IV). As the polymer having a carboxy group represented by the Formula (IV) used in this step, a polymer which has previously formed a salt with a metal or an organic base may be used. As the condensing agent to be used for the condensation reaction, for example, 1-ethyl-3-(3-dimethylaminopropyl)carbodiimide hydrochloride (EDC or WSC), 4-(4,6-dimethoxy-1,3,5-triazin-2-yl)-4-methylmorpholinium chloride (DMT-MM), tetramethylfluoroformamidinium hexafluorophosphate (TFFH), bis(tetramethylene)fluoroformamidinium hexafluorophosphate (BTFFH), or the like can be used. Further, in a case where the carboxy group of the acidic polysaccharide is derivatized into an active ester such as N-hydroxysuccinimide ester or p-nitrophenyl ester, it is not necessary to add a condensing agent, and condensation can also be performed by only mixing with the amine form represented by Formula (III), or if necessary, adding a base.

This step is preferably performed in a solvent, and for example, water or an organic solvent such as methylene chloride, chloroform, dichloroethane, toluene, ethyl acetate, acetone, dimethylformamide, formamide, N-methylpyrrolidone, acetonitrile, tetrahydrofuran, dioxane, diethyl ether, dimethoxyethane, dimethylsulfoxide, methanol, ethanol, 1-propanol, 2-propanol, or ethylene glycol can be used. Further, those organic solvents and water are mixed at an arbitrary ratio and can also be used as a mixed solvent.

The present invention relates to inventions specified by the following items.

1. A compound represented by Formula (I) or a pharmaceutically acceptable salt thereof;

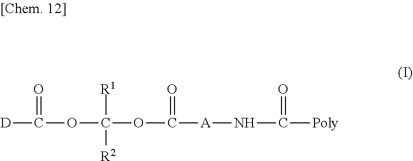

in Formula (I), D represents a residue of a primary or secondary amine compound DH excluding a hydrogen atom of a primary or secondary amino group; R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a substituted or unsubstituted bivalent hydrocarbon group and may contain one or more hetero atoms at a position except for both ends which are bonded to —C(═O)— or —NH—, the hetero atoms are each independently selected from the group consisting of —O—, —NH— which may have a substituent, and —S—; any two or three groups of R¹, R², and A may combine together to form a ring; Poly represents an acidic polysaccharide residue, and —C(═O)— adjacent to Poly is derived from a carboxy group of the acidic polysaccharide.

2. The compound according to the above 1. or a pharmaceutically acceptable salt thereof, wherein Formula (I) is represented by the following Formula (II):

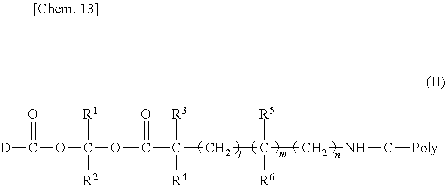

in the formula, D, R¹, R², and Poly are as defined in the above 1.; R³, R⁴, R³ and R⁶ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three groups of R¹, R², R³, R⁴, R⁵ and R⁶ may combine together to form a ring; 1 and n are each independently 0, 1, or 2, and m is 0 or 1.

3. The compound according to the above 1. or 2. or a pharmaceutically acceptable salt thereof, wherein in Formula (I) or (II); R¹, R², R³, R⁴, R⁵, and R⁶ are each independently a hydrogen atom; a substituted or unsubstituted linear or branched alkyl group having carbon number of 1 to 6; a substituted or unsubstituted cycloalkyl group having carbon number of 3 to 8; a substituted or unsubstituted linear or branched alkenyl group having carbon number of 2 to 6; a substituted or unsubstituted cycloalkenyl group having carbon number of 3 to 8; a substituted or unsubstituted linear or branched alkynyl group having carbon number of 2 to 6; a substituted or unsubstituted monocyclic or polycyclic aromatic group having carbon number of 6 to 14; or a substituted or unsubstituted 3- to 8-membered heterocyclic group containing at least one of a nitrogen atom, an oxygen atom, or a sulfur atom as a ring-constituting atom.

4. The compound according to any one of the above 1. to 3. or a pharmaceutically acceptable salt thereof,

wherein in Formula (I) or (II), a substituent of alkyl, a substituent of cycloalkyl group, a substituent of alkenyl group, a substituent of cycloalkenyl group, a substituent of alkynyl group, a substituent of aromatic group, and a substituent of heterocyclic group in the groups represented by R¹, R², R³, R⁴, R⁵, and R⁶ are groups selected from a hydroxyl group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclylthio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an Rx(Ry)N group, and an Rx(Ry)(Rz)N⁺ group (herein, Rx, Ry, and Rz each independently represent a hydrogen atom, an alkyl group, a cycloalkyl group, an alkenyl group, a cycloalkenyl group, an alkynyl group, an aromatic hydrocarbon group, or a heterocyclic group; in addition, Rx, Ry, and Rz may be bonded to each other to form a saturated or unsaturated hetero ring, and the ring is also capable of forming a condensed ring or a spiro ring with an aliphatic ring or a hetero ring and also capable of forming a condensed ring with an aromatic ring).

5. The compound according to any one of the above 1. to 4., or a pharmaceutically acceptable salt thereof, wherein in Formula (I) or (II), Poly is a glycosaminoglycan residue.

6. The compound according to any one of the above 1. to 4., or a pharmaceutically acceptable salt thereof, wherein in Formula (I) or (II), Poly is a residue of chondroitin, chondroitin sulfate or hyaluronic acid.

7. The compound according to the above 1., represented by the following Formula (XX), or a pharmaceutically acceptable salt thereof;

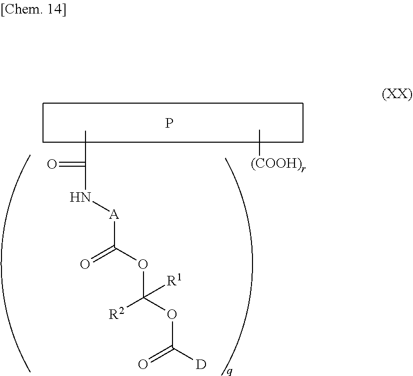

in Formula (XX), D, R¹, R², and A are as defined in the above 1.,

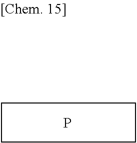

a moiety represented by P represents a polymer excluding a group derived from a carboxy group and the carboxy group of the acidic polysaccharide; q represents a number of a compound condensed with the polymer; r represents a number of substitution of the carboxy group.

8. The compound according to the above 7. or a pharmaceutically acceptable salt thereof, in which the compound represented by the Formula (XX) is a compound represented by the following Formula (XXX):

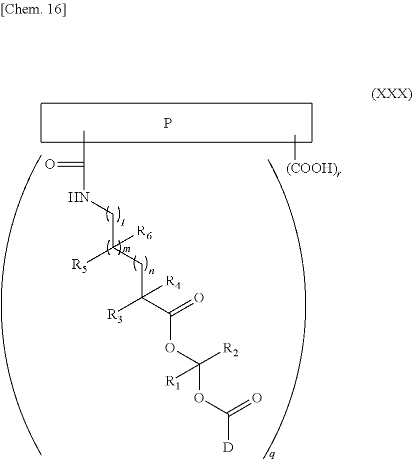

in Formula (XXX), D, R¹, and R² are as defined in the above 1., R⁴, R⁵, and R⁶ are as defined in the above 2., and q and r are as described in the above 7.:

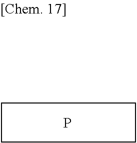

a moiety represented by P is a polymer excluding a group derived from a carboxy group and the carboxy group.

9. A method for producing a compound represented by the following Formula (I) or a pharmaceutically acceptable salt thereof, the method comprising a step of condensing a compound represented by the following Formula (III) and an acidic polysaccharide represented by the following Formula (IV):

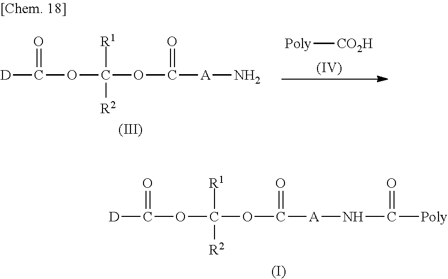

Wherein, D, A, R¹, R², and Poly in Formulas (I), (III), and (IV) are as defined in the above 1., and the compound represented by (III) may form a salt with an inorganic acid or an organic acid.

10. A method for producing a conjugate, the method including a step of bonding a primary or secondary amine compound to an acidic polysaccharide via a linker represented by the following Formula (V):

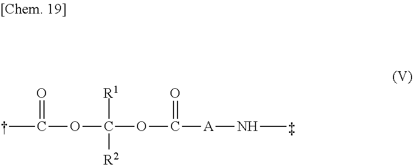

wherein, R¹, R², and A in the (V) are as defined in the above 1., symbol t represents a node with a moiety of a nitrogen atom of an amino group of the primary or secondary amine compound, and symbol $ represents a node with a moiety of a carbonyl carbon derived from the acidic polysaccharide.

11. The method for producing a conjugate according to the above 10., wherein the linker is represented by the following Formula (VI):

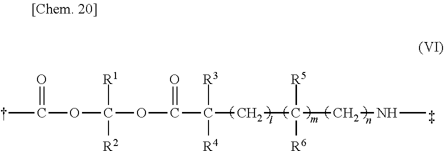

wherein, R¹, R², R³, R⁴, R⁵, R⁶, 1, m, and n in the (VI) are as defined in the above 2., and symbols t and $ are as defined in the above 8.

### EXAMPLES

Hereinafter, the present invention will be described in detail by means of Reference Examples and Examples; however the scope of the present invention is not limited to those examples.

### Reference Example 1

### 2-[[3-[[(etilefrine-carbonyl)oxy]methoxy]-3-oxopropyl]amino]-2-oxoethyl-methoxypolyethylene glycol (20,000) conjugate

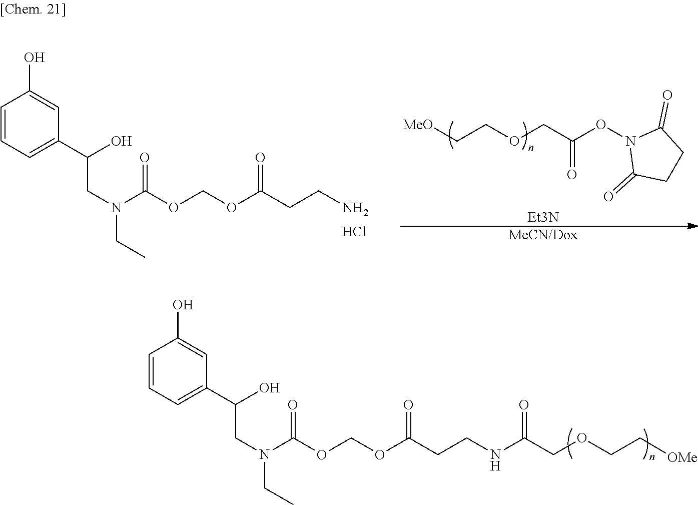

551 mg (0.03 mmol) of methoxypolyethylene glycol (20,000) acetic acid N-hydroxysuccinimide ester and 28 mg (0.28 mmol) of triethylamine were dissolved in 1.5 mL of acetonitrile. A mixed solution of 100 mg (0.3 mmol) of N-ethyl-N-[2-hydroxy-2-(3-hydroxyphenyl)ethyl]carbamic acid (3-amino-1-oxopropoxy)methyl ester hydrochloride in acetonitrile/dioxane (1/1) was added to the reaction solution and stirred at room temperature overnight. The solvent was distilled off under reduced pressure, the residue was dissolved by adding 2 mL of acetonitrile. Under stirring the solution, isopropyl alcohol was slowly added dropwise until before the reaction solution became cloudy. The suspension was added dropwise to 25 mL of isopropyl alcohol under stirring, and the deposited precipitates were filtered. The precipitates on the filter were washed two times with isopropyl alcohol and one time with diethyl ether. The precipitates were dried at room temperature overnight with a vacuum pump to obtain 507.2 mg of the title compound. Based on values of integral in ¹H-NMR, the introduction ratio of etilefrine was 68.6%.

### Example 1

### Carbonic acid chloromethyl 4-nitrophenyl ester

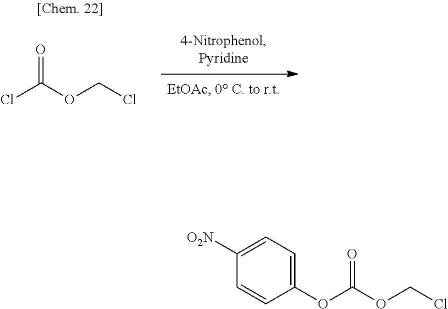 [SMILES: O=C(Cl)OCCl ; O=C(OCCl)Oc1ccc([N+](=O)[O-])cc1]

Under cooling on ice, an ethyl acetate solution of 5.7 g (44.3 mmol) of chloromethyl chloroformate was added to an ethyl acetate solution of 5.6 g (40.3 mmol) of p-nitrophenol and 3.2 g (40.3 mmol) of pyridine. The reaction solution was stirred at the same temperature for 30 minutes and then stirred at room temperature for 2 hours. Water was added to the reaction solution and stirred, and then the organic layer was isolated. The organic layer was washed with a 10% potassium hydrogen sulfate aqueous solution, a saturated sodium hydrogen carbonate aqueous solution, and saturated saline. The organic layer was dried over anhydrous sodium sulfate, and the solvent was distilled off under reduced pressure to obtain 8.6 g (83%) of the title compound.

¹H-NMR (CDCl₃, δ): 5.85 (2H, s), 7.43 (2H, d, J=9 Hz), 8.31 (2H, d, J=9 Hz)

### Example 2

### Carbonic acid iodomethyl 4-nitrophenyl ester

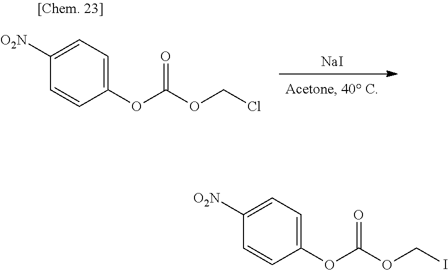 [SMILES: O=C(OCCl)Oc1ccc([N+](=O)[O-])cc1 ; "NaI" ; O=C(OCI)Oc1ccc([N+](=O)[O-])cc1]

An acetone suspension of 11.07 g (73.9 mmol) of sodium iodide and 8.55 g (36.9 mmol) of carbonic acid chloromethyl 4-nitrophenyl ester was stirred at 40° C. overnight. The reaction solution was condensed under reduced pressure, and diethyl ether was added to the residue and stirred. The insoluble matter was removed by filtration, and the organic layer was washed with water, a 10% sodium thiosulfate aqueous solution, and saturated saline. The organic layer was dried over anhydrous sodium sulfate, and the solvent was distilled off under reduced pressure to obtain 8.78 g (74%) of the title compound.

¹H-NMR (CDCl₃, δ): 6.07 (2H, s), 7.42 (2H, d, J=9 Hz), 8.30 (2H, d, J=9 Hz)

### Example 3

### Carbonic acid 1-chloroethyl 4-nitrophenyl ester

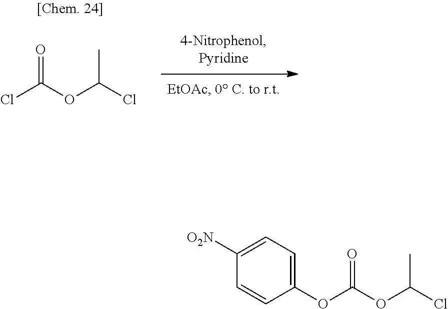

Under cooling on ice, an ethyl acetate solution of 6.3 g (44.3 mmcl) of 1-chloroethyl chloroformate was added to an ethyl acetate solution of 5.6 g (40.3 mmol) of p-nitrophenol and 3.2 g (40.3 mmol) of pyridine. The reaction solution was stirred at the same temperature for 30 minutes and then stirred at room temperature for 2 hours. Water was added to the reaction solution and stirred, and then the organic layer was isolated. The organic layer was washed with a 10% sodium thiosulfate aqueous solution, a 1 M sodium hydroxide aqueous solution, and saturated saline. The organic layer was dried over anhydrous sodium sulfate, and the solvent was distilled off under reduced pressure to obtain 7.5 g (76%) of the title compound.

¹H-NMR (CDCl₃, δ): 1.93 (3H, d, J=6 Hz), 6.51 (1H, q, J=6 Hz), 7.43 (2H, d, J=9 Hz), 8.30 (2H, d, J=9 Hz)

### Example 4

### Carbonic acid 1-iodoethyl 4-nitrophenyl ester

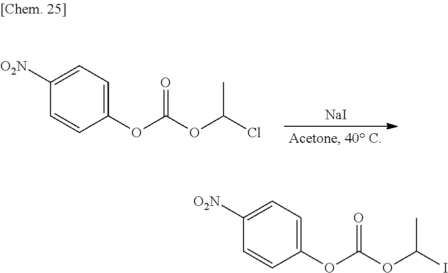 [SMILES: CC(Cl)OC(=O)Oc1ccc([N+](=O)[O-])cc1 ; "NaI" ; CC(I)OC(=O)Oc1ccc([N+](=O)[O-])cc1]

An acetone suspension of 6.10 g (40.7 mmol) of sodium iodide and 5.00 g (20.4 mmol) of carbonic acid 1-chloroethyl 4-nitrophenyl ester was stirred at 40° C. overnight. The reaction solution was condensed under reduced pressure, diethyl ether was added to the residue, and the organic layer was washed with water, a 10% sodium thiosulfate aqueous solution, and saturated saline. The organic layer was dried over anhydrous sodium sulfate, and then the solvent was distilled off under reduced pressure. The residue was purified by silica gel column chromatography (5% to 40% ethyl acetate/hexane) to obtain 3.00 g (molar ratio 2:3) (21%) of a mixture of the title compound, a starting material, and carbonic acid 1-chloroethyl 4-nitrophenyl ester.

¹H-NMR (CDCl₃, δ): 2.33 (3H, d, J=6 Hz), 6.84 (1H, q, J=6 Hz), 7.43 (2H, d, J=9 Hz), 8.30 (2H, d, J=9 Hz)

### Example 5

### N-[(1,1-dimethylethoxy)carbonyl]-β-alanine[[(4-nitrophenoxy)carbonyl]oxy]methyl ester

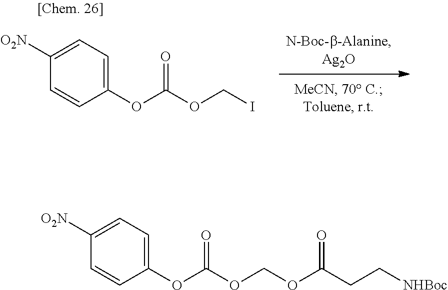 [SMILES: O=C(OCI)Oc1ccc([N+](=O)[O-])cc1 ; "N-Boc-b-Alanine,Ag2O" ; CC(C)(C)OC(=O)NCCC(=O)OCOC(=O)Oc1ccc([N+](=O)[O-])cc1]

3.48 g (15.0 mmol) of silver oxide (I) was added to a solution of 4.73 g (25.0 mmol) of N-[(1,1-dimethylethoxy)carbonyl]-β-alanine in 60 mL of acetonitrile. Under an argon stream, 30 mL of water was added to the mixed liquid and stirred at 70° C. for 1 hour. The reaction solution was filtered through celite, and the filtrate was condensed under reduced pressure. The residue was dried overnight with a vacuum pump to obtain 7.77 g of N-[(1,1-dimethylethoxy)carbonyl]-β-alanine silver salt. To a suspension of 2.96 g (10.0 mmol) of the obtained N-[(1,1-dimethylethoxy)carbonyl]-β-alanine silver salt in dry toluene, a solution of 1.62 g (5.0 mmol) of carbonic acid iodomethyl 4-nitrophenyl ester in dry toluene was added. The resultant solution was stirred at room temperature overnight. Ethyl acetate was added to the reaction solution, the insoluble matter was removed by filtration, and the solvent was distilled off under reduced pressure. The residue was purified by silica gel column chromatography (5% to 40% ethyl acetate/hexane) to obtain 276 mg (14%) of the title compound.

¹H-NMR (CDCl₃, δ): 1.44 (9H, s), 2.66 (2H, t, J=6 Hz), 3.39-3.50 (2H, m), 4.96 (1H, br s), 5.90 (2H, s), 7.42 (2H, d, J=9 Hz), 8.30 (2H, d, J=9 Hz)

### Example 6

### N-[(1,1-dimethylethoxy)carbonyl]-β-alanine-1-[[(4-nitrophenoxy)carbonyl]oxy]ethyl ester

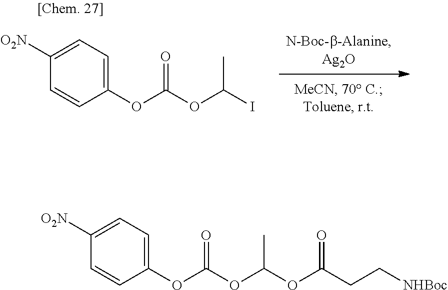 [SMILES: CC(I)OC(=O)Oc1ccc([N+](=O)[O-])cc1 ; "N-Boc-b-Alanine,Ag2O" ; CC(OC(=O)CCNC(=O)OC(C)(C)C)OC(=O)Oc1ccc([N+](=O)[O-])cc1]

3.48 g (15.0 mmol) of silver oxide (I) was added to a solution of 4.73 g (25.0 mmol) of N-[(1,1-dimethylethoxy)carbonyl]-β-alanine in 60 mL of acetonitrile. Under an argon stream, 30 mL of water was added to the mixed liquid and stirred at 70° C. for 1 hour. The reaction solution was filtered through celite, and the filtrate was condensed under reduced pressure. The residue was dried overnight with a vacuum pump to obtain 7.77 g of N-[(1,1-dimethylethoxy)carbonyl]-β-alanine silver salt. To a suspension of 2.52 g (8.5 mmol) of the obtained N-[(1,1-dimethylethoxy)carbonyl]-β-alanine silver salt in dry toluene, a solution of 1.50 g (2.1 mmol) of a mixture of carbonic acid 1-iodoethyl 4-nitrophenyl ester and carbonic acid 1-chloroethyl 4-nitrophenyl ester (mol ratio; 2:3) in dry toluene were added. The resultant solution was stirred at room temperature overnight. Ethyl acetate was added to the reaction solution, the insoluble matter was removed by filtration, and the solvent was distilled off under reduced pressure. The residue was purified by silica gel column chromatography (5% to 40% ethyl acetate/hexane) to obtain 116 mg (14%) of the title compound.

¹H-NMR (CDCl₃, δ): 1.43 (9H, s), 1.63 (3H, d, J=5 Hz), 2.61 (2H, t, J=6 Hz), 3.36-3.48 (2H, m), 4.95 (1H, br s), 6.86 (1H, q, J=5 Hz), 7.41 (2H, d, J=9 Hz), 8.29 (2H, d, J=9 Hz)

### Example 7

### N-ethyl-N-[2-hydroxy-2-(3-hydroxyphenyl)ethyl]carbamic acid chloromethyl ester

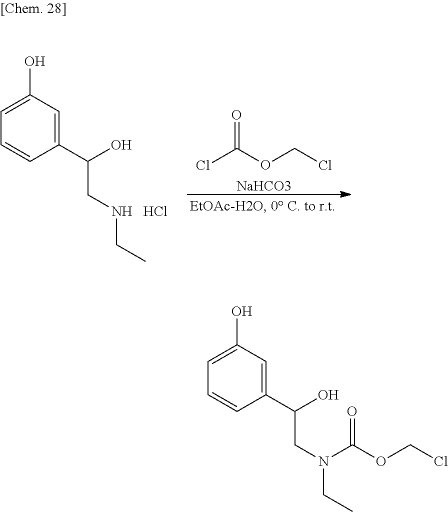

To a mixed solution of 1000 mg (4.6 mmol) of etilefrine hydrochloride and 1158 mg (13.8 mmol) of sodium hydrogen carbonate in ethyl acetate-water (5 mL: 2.5 mL), an ethyl acetate solution of 652 mg (5.1 mmol) of chloromethyl chloroformate was added dropwise under cooling on ice, and the resultant solution was stirred at room temperature for 3 hours and 30 minutes. The aqueous layer was removed, and the organic layer was washed with saturated saline, dried over anhydrous magnesium sulfate, and the solvent was distilled off under reduced pressure. The residue was purified by silica gel column chromatography (12% to 100% ethyl acetate/hexane) to obtain 575 mg (2.1 mmol) (46%) of the title compound.

¹H-NMR (CDCl₃, δ): 1.05-1.16 (3H, m), 3.20-3.33 (2H, m), 3.35-3.56 (2H, m), 4.85-4.99 (1H, m), 5.06-5.14 (1H, m), 5.75-5.87 (2H, m), 6.74-6.81 (1H, m), 6.82-6.98 (2H, m), 7.22 (1H, br t, J=8 Hz)

### Example 8

### N-ethyl-N-[2-hydroxy-2-(3-hydroxyphenyl)ethyl]carbamic acid[3-[(1,1-dimethylethoxy)carbonyl]amino-1-oxopropoxy]methyl ester

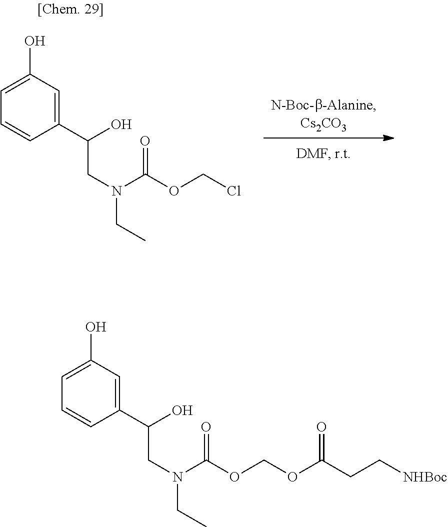 [SMILES: CCN(CC(O)c1cccc(O)c1)C(=O)OCCl ; CCN(CC(O)c1cccc(O)c1)C(=O)OCOC(=O)CCNC(=O)OC(C)(C)C]

4073 mg (12.5 mmol) of cesium carbonate was added to a methanol solution of 4730 mg (25.0 mmol) of N-[(1,1-dimethylethoxy)carbonyl]-β-alanine and stirred at room temperature for 30 minutes. The solvent was distilled off under reduced pressure, and dried overnight using a vacuum pump. 674 mg (2.1 mmol) of the obtained N-[(1,1-dimethylethoxy)carbonyl]-β-alanine cesium salt was collected and dissolved in N,N-dimethylformamide. An N,N-dimethylformamide solution of 575 mg (2.1 mmol) of N-ethyl-N-[2-hydroxy-2-(3-hydroxyphenyl)ethyl]carbamic acid chloromethyl ester was added to the reaction solution and stirred overnight. Water was added to the reaction solution and extracted with diethyl ether. The organic layer was washed with water, and then the aqueous layer was back-extracted again with diethyl ether. The obtained organic layer was combined, washed with saturated saline, and then dried over anhydrous magnesium sulfate, and the solvent was distilled off under reduced pressure. The residue was purified by silica gel column chromatography (33% to 60% ethyl acetate/hexane) to obtain 640 mg (1.5 mmol) (71%) of the title compound.

¹H-NMR (CDCl₃, δ): 1.02 (2H, br t, J=7 Hz), 1.12 (1H, br t, J=7 Hz), 1.44 (9H, br s), 2.61 (2H, t, J=7 Hz), 3.05-3.26 (1H, m), 3.27-3.47 (4H, m), 3.52 (1H, br s), 4.74-4.84 (1/2H, m), 4.92-4.98 (1/2H, m), 5.02 (1H, br s), 5.72-5.85 (2H, m), 6.73-6.82 (1H, m), 6.83-6.94 (2H, m), 7.21 (1H, br t, J=8 Hz)

### Example 9

### N-ethyl-N-[2-hydroxy-2-(3-hydroxyphenyl)ethyl]carbamic acid (3-amino-1-oxopropoxy)methyl ester hydrochloride

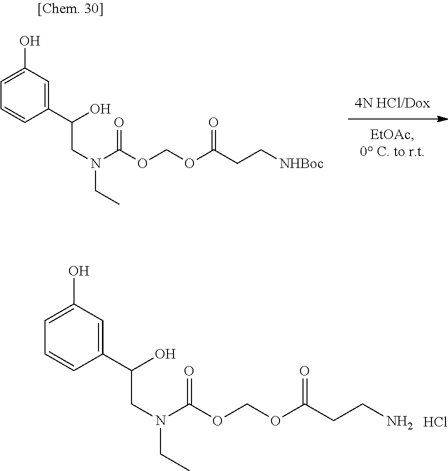 [SMILES: CCN(CC(O)c1cccc(O)c1)C(=O)OCOC(=O)CCNC(=O)OC(C)(C)C ; CCN(CC(O)c1cccc(O)c1)C(=O)OCOC(=O)CCN ; Cl]

3 mL of 4 N hydrochloric acid/dioxane solution was added to an ethyl acetate solution of 640 mg (1.5 mmol) of N-ethyl-N-[2-hydroxy-2-(3-hydroxyphenyl)ethyl]carbamic acid[3-[(1,1-dimethylethoxy)carbonyl]amino-1-oxopropoxy]methyl ester, and the reaction was left to stand still for 4 hours. The solvent was distilled off under reduced pressure, diisopropyl ether was added to the residue and stirred. The precipitated crystals were filtered and dissolved in ethanol. The solvent was distilled off under reduced pressure to obtain 507 mg (1.4 mmol) (93%) of the title compound.

¹H-NMR (CDCl₃, δ): 0.97-1.05 (3H, m), 2.73-2.80 (2H, m), 3.04 (2H, t, J=7 Hz), 3.13-3.31 (4H, m), 4.57-4.70 (11H, m), 5.39-5.46 (1H, m), 5.68 (11H, s), 5.74 (1H, s), 6.62-6.70 (1H, m), 6.70-6.80 (2H, m), 7.12 (11H, t, J=8 Hz), 8.00 (3H, br s), 9.35 (1H, s)

### Example 10

### [3-[[(etilefrine-carbonyl)oxy]methoxy]-3-oxopropyl]amino-chondroitin sulfate conjugate

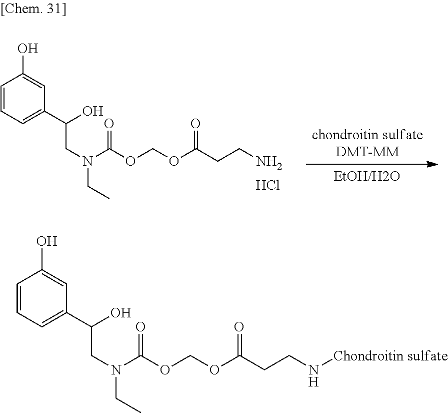

200 mg (0.4 mmol) of sodium chondroitin sulfate was dissolved in 4 mL of water, and 4 mL of ethanol was added dropwise thereto under stirring. To the mixed liquid, 50% ethanol aqueous solution of 42 mg (0.1 mmol) of N-ethyl-N-[2-hydroxy-2-(3-hydroxyphenyl)ethyl]carbamic acid (3-amino-1-oxopropoxy)methyl ester hydrochloride was added. Then, 50% ethanol aqueous solution of 54 mg (0.1 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 4 mL) until immediately before the reaction solution became cloudy. The reaction solution was added dropwise under stirring to 8 mL of 90% ethanol, and 12 mL of ethanol was added to the mixed liquid and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 211 mg of the title compound. Based on values of integral in ¹H-NMR, the introduction ratio of etilefrine per unit of whole disaccharide (glucuronic acid) of chondroitin sulfate was 22%.

### Example 11

### N-ethyl-N-[2-hydroxy-2-(3-hydroxyphenyl)ethyl]carbamic acid-1-chloro-2-methylpropyl ester

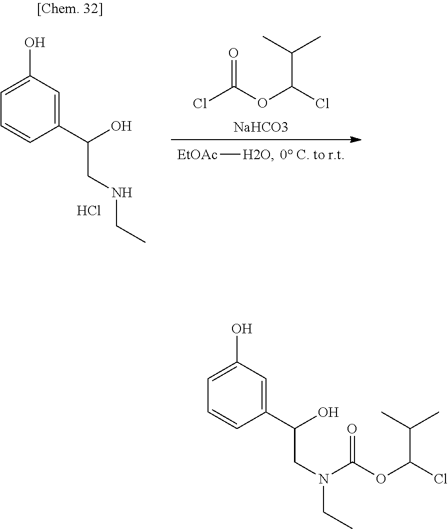

To a mixed solution of 1000 mg (4.6 mmol) of etilefrine hydrochloride and 1158 mg (13.8 mmol) of sodium hydrogen carbonate in ethyl acetate-water (5 mL: 10 mL), an ethyl acetate solution of 943 mg (5.5 mmol) of 1-chloro-2-methylpropyl chloroformate was added dropwise under cooling on ice, and the resultant solution was stirred at room temperature overnight. Under cooling on ice, an ethyl acetate solution of 351 mg (2.1 mmol) of 1-chloro-2-methylpropyl chloroformate was again added dropwise, and the reaction solution was stirred at room temperature overnight. The aqueous layer was removed, and the organic layer was washed with saturated saline, dried over anhydrous magnesium sulfate, and the solvent was distilled off under reduced pressure. The residue was purified by silica gel column chromatography (1% to 12% methanol/chloroform) and dried using a vacuum pump overnight to obtain 409 mg (1.3 mmol) (28%) of the title compound.

¹H-NMR (CDCl₃, δ): 1.01-1.17 (9H, m), 2.08-2.27 (1H, m), 3.12-3.57 (4H, m), 4.85-5.00 (1H, m), 6.33-6.44 (1H, m), 6.72-6.83 (1H, m), 6.84-6.96 (2H, m), 7.22 (1H, br t, J=8 Hz)

### Example 12

### N-ethyl-N-[2-hydroxy-2-(3-hydroxyphenyl)ethyl]carbamic acid 1-[3-[[(1,1-dimethylethoxy)carbonyl]amino]-2-methyl-1-oxopropoxy]-2-methylpropyl ester

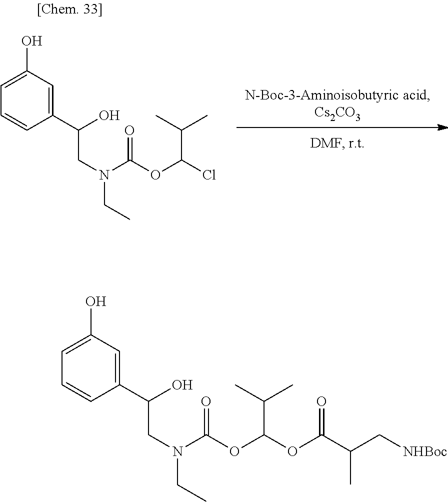 [SMILES: CCN(CC(O)c1cccc(O)c1)C(=O)OC(Cl)C(C)C ; "N-Boc-3-Aminoisobutyric acid,Cs2CO3" ; CCN(CC(O)c1cccc(O)c1)C(=O)OC(OC(=O)C(C)CNC(=O)OC(C)(C)C)C(C)C]

408 mg (1.3 mmol) of cesium carbonate was added to a methanol solution of 509 mg (2.5 mmol) of 3-[[(1,1-dimethylethoxy)carbonyl]amino]-2-methyl propanoic acid and stirred at room temperature for 10 minutes. The solvent was distilled off under reduced pressure, and dried overnight using a vacuum pump. 416 mg (1.3 mmol) was collected from the obtained 3-[[(1,1-dimethylethoxy)carbonyl]amino]-2-methyl propanoic acid cesium salt, and dissolved in N, N-dimethylformamide, an N,N-dimethylformamide solution of 409 mg (1.3 mmol) of N-ethyl-N-[2-hydroxy-2-(3-hydroxyphenyl)ethyl]carbamic acid-1-chloro-2-methylpropyl ester was added thereto and stirred overnight. 160 mg (0.5 mmol) of N-ethyl-N-[2-hydroxy-2-(3-hydroxyphenyl)ethyl]carbamic acid-1-chloro-2-methylpropyl ester was further added to the reaction solution and stirred for 3 days. Saturated sodium bicarbonate water was added to the reaction solution and extracted with diethyl ether. The obtained organic layer was washed with water and saturated saline, and then dried over anhydrous magnesium sulfate, and the solvent was distilled off under reduced pressure. The residue was purified by silica gel column chromatography (5% to 40% ethyl acetate/hexane) and dried using a vacuum pump overnight to obtain 109 mg (0.23 mmol) (18%) of the title compound.

¹H-NMR (CDCl₃, δ): 0.92-1.13 (9H, m), 1.16 (3H, t, J=7 Hz), 1.42 (9H, br s), 1.88-2.13 (2H, m), 2.72 (1H, br s), 2.87-3.48 (5H, m), 3.48-3.64 (1H, m), 4.75-4.97 (1H, m), 5.05-5.25 (1H, m), 6.52-6.65 (1H, m), 6.72-7.02 (3H, m), 7.12-7.23 (1H, m)

### Example 13

### N-ethyl-N-[2-hydroxy-2-(3-hydroxyphenyl)ethyl]carbamic acid 1-(3-amino-2-methyl-1-oxopropoxy)-2-methylpropyl ester trifluoroacetate

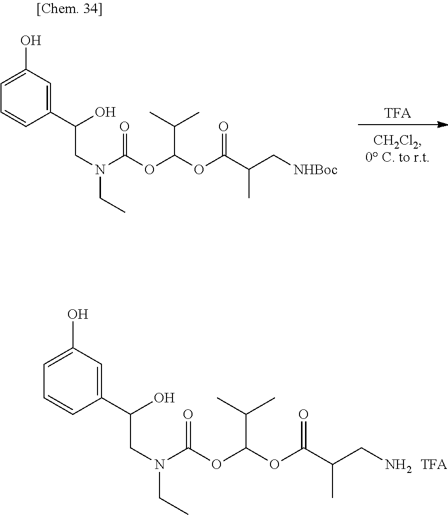 [SMILES: CCN(CC(O)c1cccc(O)c1)C(=O)OC(OC(=O)C(C)CNC(=O)OC(C)(C)C)C(C)C ; "TFA" ; CCN(CC(O)c1cccc(O)c1)C(=O)OC(OC(=O)C(C)CN)C(C)C]

0.2 mL of trifluoroacetic acid was slowly added to a methylene chloride solution of 47 mg (0.1 mmol) of N-ethyl-N-[2-hydroxy-2-(3-hydroxyphenyl)ethyl]carbamic acid 1-[3-[[(1,1-dimethylethoxy)carbonyl]amino]-2-methyl-1-oxopropoxy]-2-methylpropyl ester, and the reaction solution was left to stand still for 2 hours and 30 minutes. The solvent was distilled off under reduced pressure, diisopropyl ether was added to the residue and stirred. The precipitated crystals were filtered to obtain 25 mg (50%) of the title compound.

¹H-NMR (DMSO-d₆, δ): 0.91-1.06 (9H, m), 1.11-1.19 (3H, m), 1.97-2.08 (1H, m), 2.76-2.83 (1H, m), 2.83-2.92 (1H, m), 3.01-3.30 (5H, m), 4.57-4.67 (1H, m), 5.33-5.48 (1H, m), 6.45 (1/5H, d, J=4 Hz), 6.48 (4/5H, d, J=4 Hz), 6.61-6.85 (3H, m), 7.02-7.18 (1H, m), 7.79 (3H, br s), 9.22-9.39 (1H, m)

### Example 14

### 3-[(3,4,5-trimethoxybenzoyl)amino]-1-piperidinecarboxylic acid chloromethyl ester

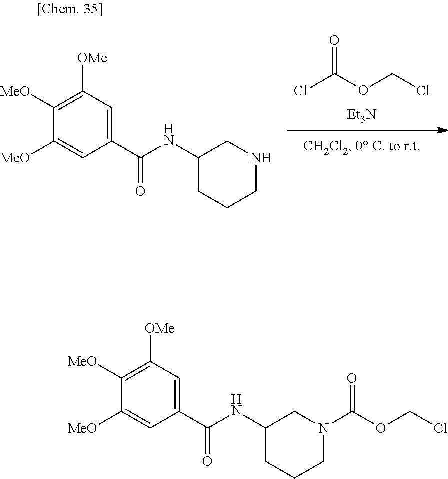 [SMILES: "Et3N" ; COc1cc(C(=O)NC2CCCNC2)cc(OC)c1OC ; O=C(Cl)OCCl ; COc1cc(C(=O)NC2CCCN(C(=O)OCCl)C2)cc(OC)c1OC]

A methylene chloride solution of 789 mg (6.1 mmol) of chloromethyl chloroformate was added under cooling on ice to a methylene chloride solution of 1500 mg (5.1 mmol) of troxipide and 619 mg (6.1 mmol) of triethylamine and stirred at room temperature overnight. The reaction solution was condensed under reduced pressure, ethyl acetate was added to the residue and washed with water, a 10% potassium hydrogen sulfate aqueous solution, and saturated saline. The organic layer was dried over anhydrous magnesium sulfate, and the solvent was distilled off under reduced pressure. The residue was purified by silica gel column chromatography (25% ethyl acetate/hexane) to obtain 1824 mg (4.7 mmol) (92%) of the title compound.

¹H-NMR (CDCl₃, δ): 1.55-1.84 (3H, m), 1.85-2.10 (1H, m), 3.29-3.79 (4H, m), 3.88 (3H, s), 3.91 (6H, s), 4.10-4.29 (1H, m), 5.61-5.75 (1H, m), 5.81-6.11 (3/2H, m), 6.39 (1/2H, br s), 6.89-7.06 (2H, m)

### Example 15

### 3-[(3,4,5-trimethoxybenzoyl)amino]-1-piperidinecarboxylic acid-1-chloroethyl ester

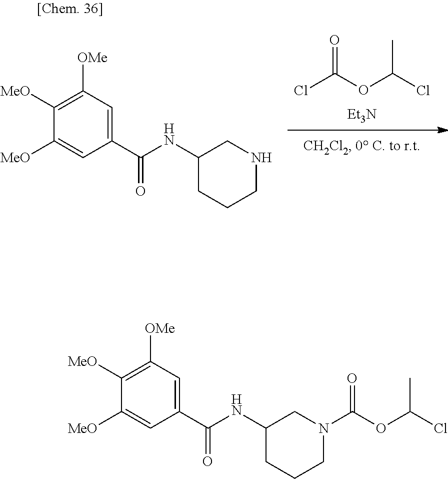

A methylene chloride solution of 874 mg (6.1 mmol) of 1-chloroethyl chloroformate was added under cooling on ice to a methylene chloride solution of 1500 mg (5.1 mmol) of troxipide and 619 mg (6.1 mmol) of triethylamine and stirred at room temperature overnight. The reaction solution was condensed under reduced pressure, ethyl acetate was added to the residue and washed with water, a 10% potassium hydrogen sulfate aqueous solution, and saturated saline. The organic layer was dried over anhydrous magnesium sulfate, and the solvent was distilled off under reduced pressure. The residue was purified by silica gel column chromatography (1% to 10% methanol/chloroform) to obtain 1146 mg (2.9 mmol) (56%) of the title compound.

¹H-NMR (CDCl₃, δ): 1.64-2.13 (7H, m), 3.14-3.31 (1H, m), 3.36-3.66 (2H, m), 3.81-3.97 (10H, m), 4.10-4.30 (1H, m), 5.88-6.14 (1/2H, m), 6.46-6.71 (3/2H, m), 6.90-7.10 (2H, m)

### Example 16

### 3-[(3,4,5-trimethoxybenzoyl)amino]-1-piperidinecarboxylic acid[3-[[(1,1-dimethylethoxy)carbonyl]amino]-1-oxopropoxy]methyl ester

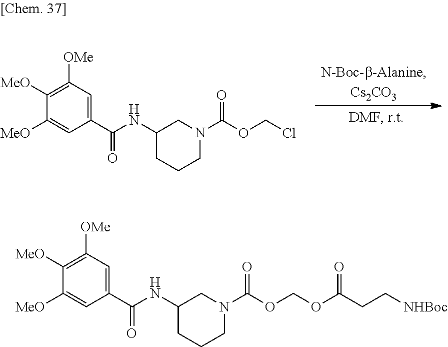 [SMILES: COc1cc(C(=O)NC2CCCN(C(=O)OCCl)C2)cc(OC)c1OC ; COc1cc(C(=O)NC2CCCN(C(=O)OCOC(=O)CCNC(=O)OC(C)(C)C)C2)cc(OC)c1OC]

4073 mg (12.5 mmol) of cesium carbonate was added to a methanol solution of 4730 mg (25.0 mmol) of N-[(1,1-dimethylethoxy)carbonyl]-β-alanine and stirred at room temperature for 30 minutes. The solvent was distilled off under reduced pressure, and dried overnight using a vacuum pump. 643 mg (2.0 mmol) was collected from the obtained N-[(1,1-dimethylethoxy)carbonyl]-β-alanine cesium salt and dissolved in N,N-dimethylformamide. An N,N-dimethylformamide solution of 775 mg (2.0 mmol) of 3-[(3,4,5-trimethoxybenzoyl)amino]-1-piperidinecarboxylic acid chloromethyl ester was added to the reaction solution and stirred overnight. Water was added to the reaction solution and extracted with diethyl ether. The organic layer was washed with water, and then the aqueous layer was back-extracted again with diethyl ether. The obtained organic layer was washed with saturated saline, and then dried over anhydrous magnesium sulfate, and the solvent was distilled off under reduced pressure. The residue was purified by silica gel column chromatography (33% ethyl acetate/hexane) to obtain 763 mg (1.4 mmol) (71%) of the title compound.

¹H-NMR (CDCl₃, δ): 1.42 (9H, s), 1.56-1.84 (2H, m), 1.86-2.10 (2H, m), 2.54 (2H, br s), 3.15-3.45 (4H, m), 3.55-3.79 (2H, m), 3.88-3.93 (9H, m), 4.05-4.27 (1H, m), 4.93 (1/2H, br s), 5.10 (1/2H, br s), 5.69-6.00 (5/2H, m), 6.37 (1/2H, br s), 6.90-7.05 (2H, m)

### Example 17

### 3-[(3,4,5-trimethoxybenzoyl)amino]-1-piperidinecarboxylic acid[[2-[[(1,1-dimethylethoxy)carbonyl]amino]acetyl]oxy]methyl ester

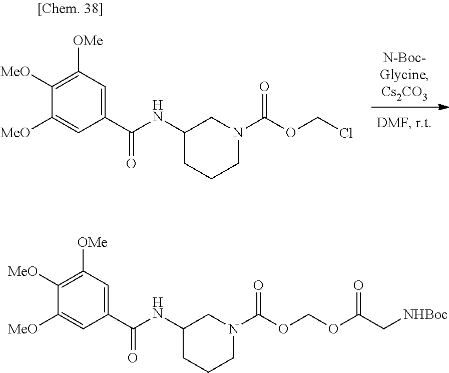 [SMILES: COc1cc(C(=O)NC2CCCN(C(=O)OCCl)C2)cc(OC)c1OC ; "N-Boc-Glycine,Cs2CO3" ; COc1cc(C(=O)NC2CCCN(C(=O)OCOC(=O)CNC(=O)OC(C)(C)C)C2)cc(OC)c1OC]

2037 mg (6.3 mmol) of cesium carbonate was added to a methanol solution of 2190 mg (12.5 mmol) of N-[(1,1-dimethylethoxy)carbonyl]-glycine and stirred at room temperature for 10 minutes. The solvent was distilled off under reduced pressure, and dried overnight using a vacuum pump. 159 mg (0.5 mmol) was collected from the obtained N-[(1,1-dimethylethoxy)carbonyl]-glycine cesium salt, and dissolved in N,N-dimethylformamide. An N,N-dimethylformamide solution of 200 mg (0.5 mmol) of 3-[(3,4,5-trimethoxybenzoyl)amino]-1-piperidinecarboxylic acid chloromethyl ester was added to the reaction solution and stirred overnight. Saturated sodium bicarbonate water was added to the reaction solution and extracted with diethyl ether. The obtained organic layer was washed with water and saturated saline, and then dried over anhydrous magnesium sulfate, and the solvent was distilled off under reduced pressure. The residue was purified by silica gel column chromatography (5% to 80% ethyl acetate/hexane) to obtain 113 mg (0.2 mmol) (40%) of the title compound.

¹H-NMR (CDCl₃, 8): 1.43 (9H, s), 1.56-1.84 (2H, m), 1.86-2.09 (2H, m), 3.14-3.43 (2H, m), 3.52-3.80 (2H, m), 3.84-3.97 (11H, m), 4.04-4.27 (1H, m), 4.88-5.15 (1H, m), 5.70-6.01 (5/2H, m), 6.37 (1/2 H, br s), 6.90-7.06 (2H, m)

### Example 18

### 3-[(3,4,5-trimethoxybenzoyl)amino]-1-piperidinecarboxylic acid 1-[3-[[(1,1-dimethylethoxy)carbonyl]amino]-1-oxopropoxy]ethyl ester

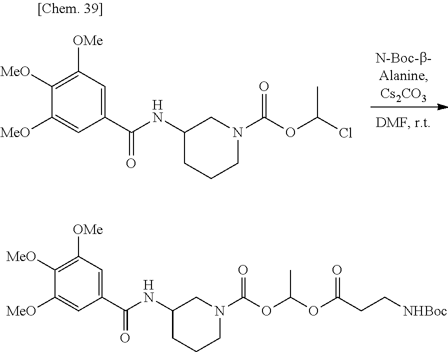 [SMILES: COc1cc(C(=O)NC2CCCN(C(=O)OC(C)Cl)C2)cc(OC)c1OC ; "N-Boc-b-Alanine,Cs2CO3" ; COc1cc(C(=O)NC2CCCN(C(=O)OC(C)OC(=O)CCNC(=O)OC(C)(C)C)C2)cc(OC)c1OC]

4073 mg (12.5 mmol) of cesium carbonate was added to a methanol solution of 4730 mg (25.0 mmol) of N-[(1,1-dimethylethoxy)carbonyl]-β-alanine and stirred at room temperature for 30 minutes. The solvent was distilled off under reduced pressure, and dried overnight using a vacuum pump. 481 mg (1.5 mmol) of the obtained N-[(1,1-dimethylethoxy)carbonyl]-β-alanine cesium salt was collected and dissolved in N,N-dimethylformamide. An N,N-dimethylformamide solution of 600 mg (1.5 mmol) of 3-[(3,4,5-trimethoxybenzoyl)amino]-1-piperidinecarboxylic acid-1-chloroethyl ester was added to the reaction solution and stirred overnight. Saturated sodium bicarbonate water was added to the reaction solution and extracted with diethyl ether. The obtained organic layer was washed with water and saturated saline, and then dried over anhydrous magnesium sulfate, and the solvent was distilled off under reduced pressure. The residue was purified by silica gel column chromatography (30% to 80% ethyl acetate/hexane) to obtain 231 mg (0.4 mmol) (28%) of the title compound.

¹H-NMR (CDCl₃, δ): 1.37-1.46 (9H, m), 1.51 (3H, d, J=5 Hz), 1.57-2.13 (4H, m), 2.28-2.62 (2H, m), 3.06-3.83 (6H, m), 3.87-3.93 (9H, m), 4.16 (1H, br s), 4.87-5.20 (1H, m), 6.10 (1/2H, br s), 6.44 (1/2H, br s), 6.79 (1H, br s), 7.01 (2H, s)

### Example 19

### 3-[(3,4,5-trimethoxybenzoyl)amino]-1-piperidinecarboxylic acid 1-[[2-[[(1,1-dimethylethoxy)carbonyl]amino]acetyl]oxy]ethyl ester

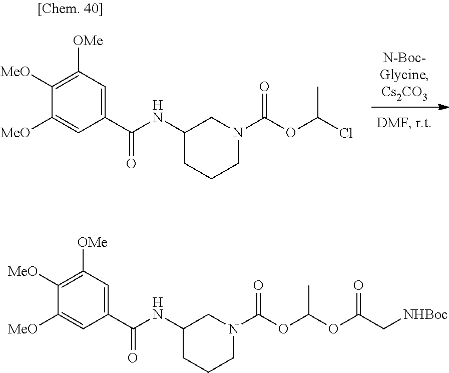 [SMILES: COc1cc(C(=O)NC2CCCN(C(=O)OC(C)Cl)C2)cc(OC)c1OC ; "N-Boc-Glycine,Cs2CO3" ; COc1cc(C(=O)NC2CCCN(C(=O)OC(C)OC(=O)CNC(=O)OC(C)(C)C)C2)cc(OC)c1OC]

2037 mg (6.3 mmol) of cesium carbonate was added to a methanol solution of 2190 mg (12.5 mmol) of N-[(1,1-dimethylethoxy)carbonyl]-glycine and stirred at room temperature for 10 minutes. The solvent was distilled off under reduced pressure, and dried overnight using a vacuum pump. 238 mg (0.8 mmol) was collected from the obtained N-[(1,1-dimethylethoxy)carbonyl]-glycine cesium salt, and dissolved in N,N-dimethylformamide. An N,N-dimethylformamide solution of 300 mg (0.8 mmol) of 3-[(3,4,5-trimethoxybenzoyl)amino]-1-piperidinecarboxylic acid-1-chloroethyl ester was added to the reaction solution and stirred overnight. Saturated sodium bicarbonate water was added to the reaction solution and extracted with diethyl ether. The obtained organic layer was washed with water and saturated saline, and then dried over anhydrous magnesium sulfate, and the solvent was distilled off under reduced pressure to obtain 213 mg (0.4 mmol) (51%) of the title compound.

¹H-NMR (CDCl₃, δ): 1.43 (9H, br s), 1.52 (3H, d, J=5 Hz), 1.57-2.08 (4H, m), 3.26-3.73 (4H, m), 3.75-3.99 (1H, m), 4.09-4.21 (11H, m), 4.80-5.02 (1H, m), 6.06 (1/2H, br s), 6.35 (1/2H, br s), 6.79-6.90 (1H, m), 7.00 (2H, s)

### Example 20

### 3-[(3,4,5-trimethoxybenzoyl)amino]-1-piperidinecarboxylic acid[(3-amino-1-oxopropoxy)]methyl ester hydrochloride

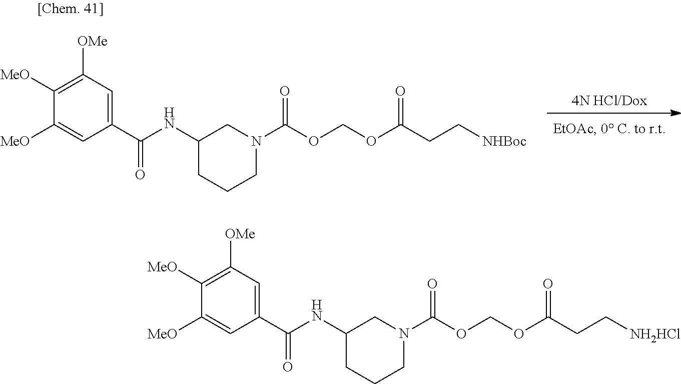

2 mL of 4 N hydrochloric acid/dioxane solution was added to an ethyl acetate solution of 763 mg (1.4 mmol) of 3-[(3,4,5-trimethoxybenzoyl)amino]-1-piperidinecarboxylic acid[[[3-[(1,1-dimethylethoxy)carbonyl]amino]-1-oxopropoxy]methyl ester, and the reaction solution was left to stand still for 3 hours. The solvent was distilled off under reduced pressure, diisopropyl ether was added to the residue and stirred. The precipitated crystals were filtered to obtain 703 mg (quantitative) of the title compound.

¹H-NMR (DMSO-d₆, δ): 1.39-1.53 (1H, m), 1.54-1.67 (1H, m), 1.73-1.85 (1H, m), 1.89-1.99 (1H, m), 2.75 (2H, br t, J=7 Hz), 2.79-2.93 (2H, m), 3.04 (2H, t, J=7 Hz), 3.71 (3H, s), 3.77-3.94 (8H, m), 3.96-4.12 (1H, m), 5.74 (2H, s), 7.18 (2H, s), 7.88 (3H, br s), 8.30 (1H, br s)

### Example 21

### 3-[(3,4,5-trimethoxybenzoyl)amino]-1-piperidinecarboxylic acid[(2-aminoacetyl)oxy]methyl ester hydrochloride

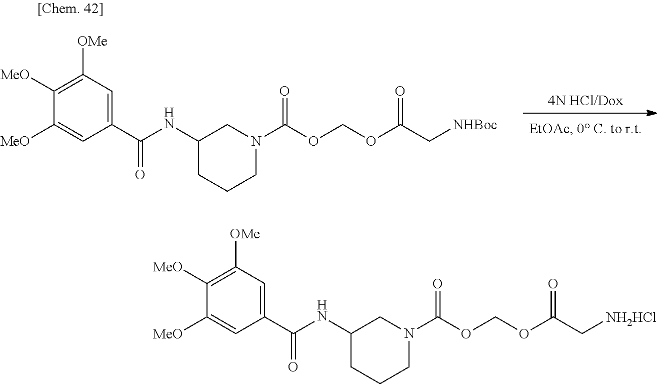

0.75 mL of 4 N hydrochloric acid/dioxane solution was added to an ethyl acetate solution of 113 mg (0.2 mmol) of 3-[(3,4,5-trimethoxybenzoyl)amino]-1-piperidinecarboxylic acid[[2-[[(1,1-dimethylethoxy)carbonyl]amino]acetyl]oxy]methyl ester, and the reaction solution was left to stand still for 3 hours. The solvent was distilled off under reduced pressure, diisopropyl ether was added to the residue and stirred. The precipitated crystals were filtered to obtain 88 mg (91%) of the title compound.

¹H-NMR (DMSO-d₆, δ): 1.40-1.53 (1H, m), 1.54-1.66 (1H, m), 1.74-1.85 (1H, m), 1.90-1.98 (1H, m), 2.71-2.94 (2H, m), 3.71 (3H, s), 3.79-3.95 (10H, m), 3.97-4.12 (1H, m), 5.83 (2H, br s), 7.18 (2H, s), 8.11 (3H, br s), 8.25-8.36 (1H, m)

### Example 22

### [3-[[(troxipide-carbonyl)oxy]methoxy]-3-oxopropyl]amino-chondroitin sulfate conjugate

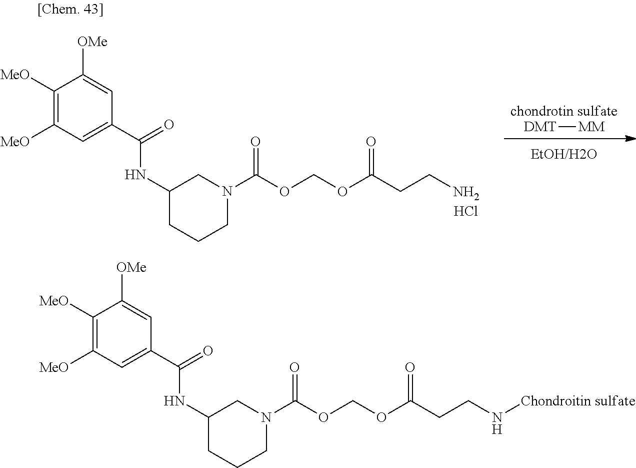

200 mg (0.39 mmol) of sodium chondroitin sulfate was dissolved in 4 mL of water, and 4 mL of ethanol was added dropwise thereto under stirring. To the mixed liquid, 50% ethanol aqueous solution of 37 mg (0.08 mmol) of 3-[(3,4,5-trimethoxybenzoyl)amino]-1-piperidinecarboxylic acid[(3-amino-1-oxopropoxy)]methyl ester hydrochloride was added. Then, 50% ethanol aqueous solution of 36 mg (0.08 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 4 mL) until immediately before the reaction solution became cloudy. The reaction solution was added dropwise under stirring to 8 mL of 90% ethanol, and 12 mL of ethanol was added to the mixed liquid and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 205 mg of the title compound. Based on values of integral in ¹H-NMR, the introduction ratio of troxipide per unit of whole disaccharide (glucuronic acid) of chondroitin sulfate was 19%.

### Example 23

### [2-[[(troxipide-carbonyl)oxy]methoxy]-2-oxoethyl]amino-chondroitin sulfate conjugate

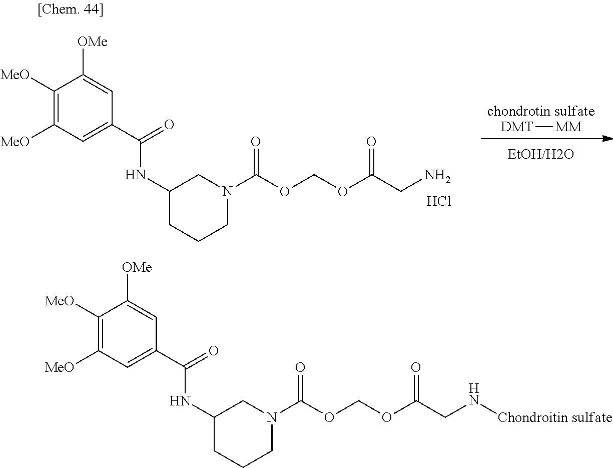

200 mg (0.39 mmol) of sodium chondroitin sulfate was dissolved in 4 mL of water, and 4 mL of ethanol was added dropwise thereto under stirring. To the mixed liquid, 50% ethanol aqueous solution of 36 mg (0.08 mmol) of 3-[(3,4,5-trimethoxybenzoyl)amino]-1-piperidinecarboxylic acid[(2-aminoacetyl)oxy]methyl ester hydrochloride was added. Then, 50% ethanol aqueous solution of 36 mg (0.08 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 4 mL) until immediately before the reaction solution became cloudy. The reaction solution was added dropwise under stirring to 8 mL of 90% ethanol, and 12 mL of ethanol was added to the mixed liquid and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 211 mg of the title compound. Based on values of integral in ¹H-NMR, the introduction ratio of troxipide per unit of whole disaccharide (glucuronic acid) of chondroitin sulfate was 27%.

### Example 24

### N-[2-(2-methoxyphenyl)-1-methylethyl]-N-methyl carbamic acid chloromethyl ester

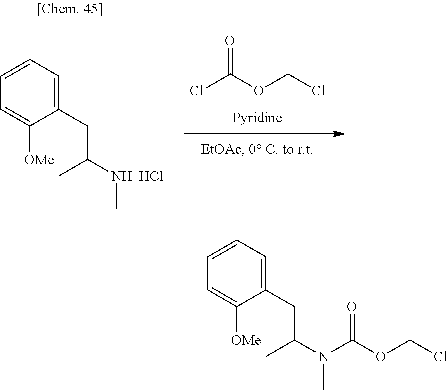 [SMILES: CNC(C)Cc1ccccc1OC ; Cl ; O=C(Cl)OCCl ; COc1ccccc1CC(C)N(C)C(=O)OCCl]

An ethyl acetate solution of 986 mg (7.7 mmol) of chloromethyl chloroformate was added under cooling on ice to an ethyl acetate solution of 1500 mg (7.0 mmol) of methoxyphenamine and 1210 mg (15.3 mmol) of pyridine and stirred at room temperature for 3 hours. The reaction solution was condensed under reduced pressure, ethyl acetate was added to the residue and washed with a 10% potassium hydrogen sulfate aqueous solution and saturated saline. The organic layer was dried over anhydrous magnesium sulfate, and the solvent was distilled off under reduced pressure. The residue was purified by silica gel column chromatography (30% to 80% ethyl acetate/hexane) to obtain 654 mg (2.4 mmol) (35%) of the title compound.

¹H-NMR (CDCl₃, δ): 1.17 (3H, d, J=7 Hz), 2.73-2.86 (5H, m), 3.83 (3H, s), 4.40-4.51 (1/2H, m), 4.51-4.60 (1/2H, m), 5.53-5.66 (1H, m), 5.66-5.74 (1H, m), 6.80-6.90 (2H, m), 7.02-7.12 (1H, m), 7.15-7.21 (1H, m)

### Example 25

### N-[2-(2-methoxyphenyl)-1-methylethyl]-N-methyl carbamic acid 1-chloroethyl ester

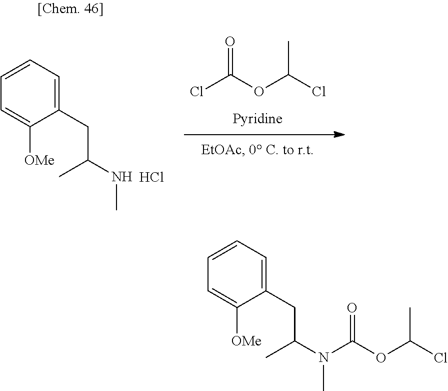

An ethyl acetate solution of 1094 mg (7.7 mmol) of 1-chloroethyl chloroformate was added under cooling on ice to an ethyl acetate solution of 1500 mg (7.0 mmol) of methoxyphenamine and 1210 mg (15.3 mmol) of pyridine and stirred at room temperature for 3 hours. The reaction solution was condensed under reduced pressure, ethyl acetate was added to the residue and washed with a 10% potassium hydrogen sulfate aqueous solution and saturated saline. The organic layer was dried over anhydrous magnesium sulfate, and the solvent was distilled off under reduced pressure. The residue was purified by silica gel column chromatography (30% to 80% ethyl acetate/hexane) to obtain 1215 mg (4.3 mmol) (61%) of the title compound.

¹H-NMR (CDCl₃, δ): 1.13-1.22 (3H, m), 1.52-1.57 (1H, m), 1.73-1.79 (2H, m), 2.70-2.86 (5H, m), 3.79-3.87 (3H, m), 4.42-4.51 (1/2H, m), 4.51-4.59 (1/2H, m), 6.37 (1/3H, q, J=6 Hz), 6.47-6.57 (2/3H, m), 6.80-6.88 (2H, m), 7.00-7.13 (1H, m), 7.14-7.22 (1H, m)

### Example 26

### N-[2-(2-methoxyphenyl)-1-methylethyl]-N-methyl carbamic acid[5-[[(1,1-dimethylethoxy)carbonyl]amino]-1-oxopentoxy]methyl ester

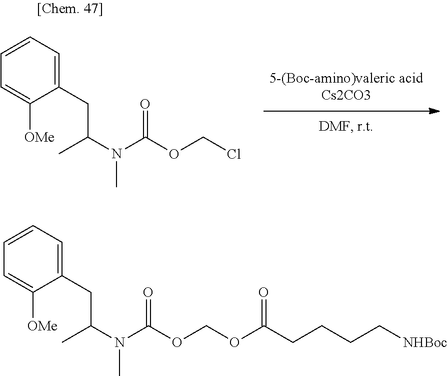 [SMILES: COc1ccccc1CC(C)N(C)C(=O)OCCl ; COc1ccccc1CC(C)N(C)C(=O)OCOC(=O)CCCCNC(=O)OC(C)(C)C]

2036.5 mg (6.3 mmol) of cesium carbonate was added to a methanol solution of 2716.0 mg (12.5 mmol) of 5-[[(1,1-dimethylethoxy)carbonyl]amino]pentanoic acid and stirred at room temperature for 10 minutes. The solvent in the mixed suspension was distilled off under reduced pressure, and dried overnight using a vacuum pump. 841 mg (2.4 mmol) was collected from the obtained 5-[[(1,1-dimethylethoxy)carbonyl]amino]pentanoic acid cesium salt and, dissolved in N,N-dimethylformamide, an N,N-dimethylformamide solution of 654 mg (2.4 mmol) of N-[2-(2-methoxyphenyl)-1-methylethyl]-N-methyl carbamic acid chloromethyl ester was added thereto and stirred overnight. Saturated sodium bicarbonate water was added to the reaction solution and extracted with diethyl ether. The organic layer was washed with water, and then the aqueous layer was back-extracted again with diethyl ether. The obtained organic layer was washed with saturated saline, and then dried over anhydrous magnesium sulfate, and the solvent was distilled off under reduced pressure. The residue was dried overnight using a vacuum pump to obtain 1002 mg (2.2 mmol) (92%) of the title compound.

¹H-NMR (CDCl₃, δ): 1.15 (3H, d, J=7 Hz), 1.42-1.46 (9H, m), 1.46-1.56 (2H, m), 1.57-1.68 (2H, m), 2.29-2.38 (2H, m), 2.71-2.83 (5H, m), 3.05-3.15 (2H, m), 3.83 (3H, s), 4.41-4.51 (1/2H, m), 4.51-4.64 (3/2H, m), 5.50-5.63 (1H, m), 5.66-5.75 (1H, m), 6.80-6.87 (2H, m), 7.00-7.12 (1H, m), 7.14-7.21 (1H, m)

### Example 27

### N-[2-(2-methoxyphenyl)-1-methylethyl]-N-methyl carbamic acid 1-[4-[[(1,1-dimethylethoxy)carbonyl]amino]-1-oxobutoxy]ethyl ester

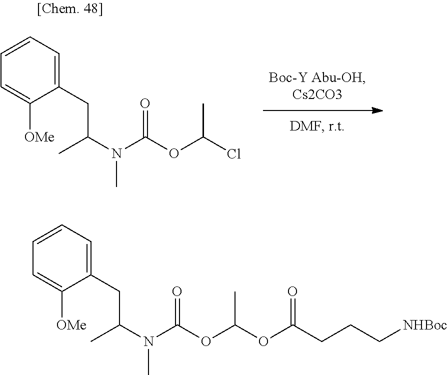 [SMILES: COc1ccccc1CC(C)N(C)C(=O)OC(C)Cl ; COc1ccccc1CC(C)N(C)C(=O)OC(C)OC(=O)CCCNC(=O)OC(C)(C)C]

2037 mg (6.3 mmol) of cesium carbonate was added to a methanol solution of 2540 mg (12.5 mmol) of 4-[[(1,1-dimethylethoxy)carbonyl]amino]butanoic acid and stirred at room temperature for 10 minutes. The solvent in the mixed suspension was distilled off under reduced pressure, and dried overnight using a vacuum pump. 352 mg (1.1 mmol) was collected from the obtained 4-[[(1,1-dimethylethoxy)carbonyl]amino]butanoic acid cesium salt and, dissolved in N,N-dimethylformamide, an N,N-dimethylformamide solution of 300 mg (1.1 mmol) of N-[2-(2-methoxyphenyl)-1-methylethyl]-N-methyl carbamic acid 1-chloroethyl ester was added thereto and stirred overnight. Saturated sodium bicarbonate water was added to the reaction solution and extracted with diethyl ether. The organic layer was washed with water, and then the aqueous layer was back-extracted again with diethyl ether. The obtained organic layer was washed with saturated saline, and then dried over anhydrous magnesium sulfate, and the solvent was distilled off under reduced pressure. The residue was purified by silica gel column chromatography (13% to 80% ethyl acetate/hexane) and dried using a vacuum pump overnight to obtain 282 mg (0.6 mmol) (59%) of the title compound.

¹H-NMR (CDCl₃, δ): 1.10-1.24 (4H, m), 1.37-1.49 (11H, m), 1.72-1.83 (2H, m), 2.23-2.38 (2H, m), 2.71-2.81 (5H, m), 3.08-3.19 (2H, m), 3.82 (2H, s), 3.84 (1H, s), 4.39-4.58 (1H, m), 4.69 (1H, br s), 6.56 (1/3H, q, J=5 Hz), 6.69-6.76 (2/3H, m), 6.81-6.88 (2H, m), 7.00-7.13 (1H, m), 7.18 (1H, td, J=8, 2 Hz)

### Example 28

### N-[2-(2-methoxyphenyl)-1-methylethyl]-N-methyl carbamic acid (5-amino-1-oxopentoxy)methyl ester hydrochloride

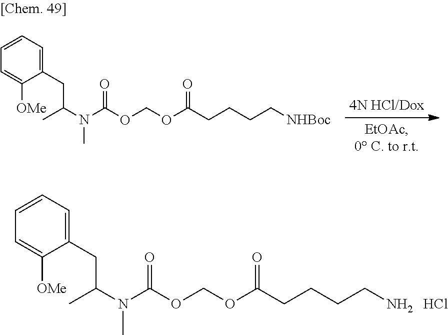 [SMILES: COc1ccccc1CC(C)N(C)C(=O)OCOC(=O)CCCCNC(=O)OC(C)(C)C ; COc1ccccc1CC(C)N(C)C(=O)OCOC(=O)CCCCN ; Cl]

1.5 mL of 4 N hydrochloric acid/dioxane solution was added to an ethyl acetate solution of 270 mg (0.6 mmol) of N-[2-(2-methoxyphenyl)-1-methylethyl]-N-methyl carbamic acid[5-[[(1,1-dimethylethoxy)carbonyl]amino]-1-oxopentoxy]methyl ester, and the reaction solution was left to stand still for 3 hours. The solvent was distilled off under reduced pressure to obtain 233 mg (quantitative) of the title compound.

¹H-NMR (DMSO-d₆, δ): 1.05-1.14 (3H, m), 1.48-1.63 (4H, m), 2.24-2.41 (2H, m), 2.64-2.74 (5H, m), 2.74-2.83 (2H, m), 3.76-3.81 (3H, m), 4.32-4.45 (1H, m), 5.39-5.53 (1H, m), 5.56-5.62 (1H, m), 6.80-6.86 (1H, m), 6.90-6.96 (1H, m), 7.02-7.09 (1H, m), 7.18 (1H, td, J=8, 2 Hz), 7.84 (3H, br s)

### Example 29

### [5-[[(methoxyphenamine-carbonyl)oxy]methoxy]-5-oxopentyl]amino-chondroitin sulfate conjugate

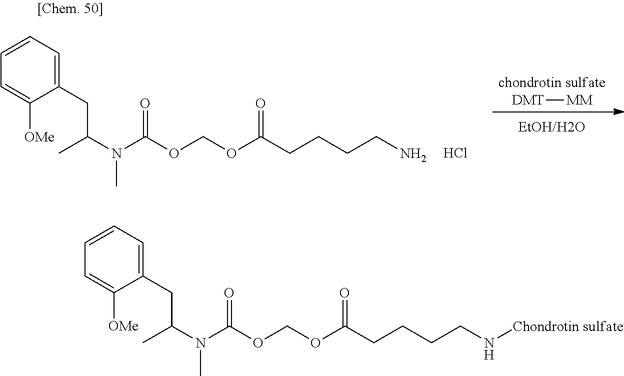

200 mg (0.39 mmol) of sodium chondroitin sulfate was dissolved in 4 mL of water, and 4 mL of ethanol was added dropwise thereto under stirring. To the mixed liquid, 50% ethanol aqueous solution of 30 mg (0.08 mmol) of N-[2-(2-methoxyphenyl)-1-methylethyl]-N-methyl carbamic acid (5-amino-1-oxopentoxy)methyl ester hydrochloride was added. Then, 50% ethanol aqueous solution of 36 mg (0.08 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 4 mL) until immediately before the reaction solution became cloudy. The reaction solution was added dropwise under stirring to 8 mL of 90% ethanol, and 12 mL of ethanol was added to the mixed liquid and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 170 mg of the title compound. Based on values of integral in ¹H-NMR, the introduction ratio of methoxyphenamine per unit of whole disaccharide (glucuronic acid) of chondroitin sulfate was 8%.

### Example 30

### N-[2-(2-methoxyphenyl)-1-methylethyl]-N-methyl carbamic acid 1-(4-amino-1-oxobutoxy)ethyl ester trifluoroacetate

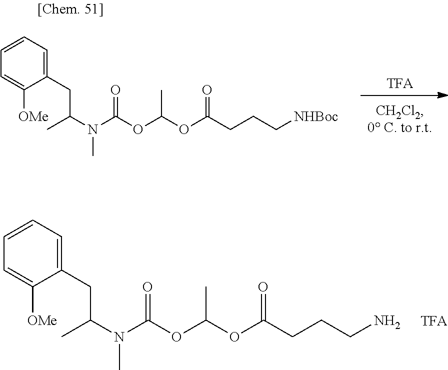 [SMILES: COc1ccccc1CC(C)N(C)C(=O)OC(C)OC(=O)CCCNC(=O)OC(C)(C)C ; COc1ccccc1CC(C)N(C)C(=O)OC(C)OC(=O)CCCN ; "TFA"]

0.5 mL of trifluoroacetic acid was slowly added to a methylene chloride solution of 282 mg (0.6 mmol) of N-[2-(2-methoxyphenyl)-1-methylethyl]-N-methyl carbamic acid 1-[4-[[(1,1-dimethylethoxy)carbonyl]amino]-1-oxobutoxy]ethyl ester, and the reaction solution was left to stand still for 2 hours. The solvent was distilled off under reduced pressure to obtain 331 mg (quantitative) of the title compound.

¹H-NMR (DMSO-d₆, δ): 1.04-1.15 (4H, m), 1.31 (1H, d, J=5 Hz), 1.38 (1H, d, J=5 Hz), 1.71-1.79 (2H, m), 2.27-2.46 (2H, m), 2.64-2.73 (5H, m), 2.75-2.85 (2H, m), 3.75-3.82 (3H, m), 4.32-4.42 (1H, m), 6.37 (1/3H, q, J=5 Hz), 6.51-6.64 (2/3H, m), 6.82-6.87 (1H, m), 6.91-6.98 (1H, m), 7.00-7.11 (1H, m), 7.14-7.21 (1H, m), 7.70 (3H, br s)

### Example 31

### [4-[1-[(methoxyphenamine-carbonyl)oxy]ethoxy]-4-oxobutyl]amino-chondroitin sulfate conjugate

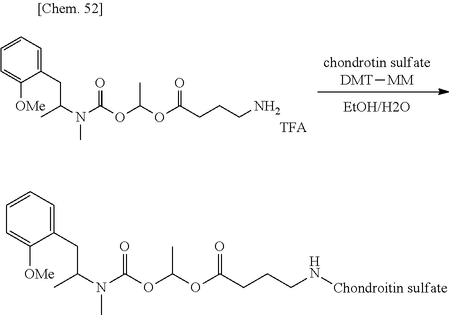

200 mg (0.39 mmol) of sodium chondroitin sulfate was dissolved in 4 mL of water, and 4 mL of ethanol was added dropwise thereto under stirring. To the mixed liquid, 50% ethanol aqueous solution of 41 mg (0.08 mmol) of N-[2-(2-methoxyphenyl)-1-methylethyl]-N-methyl carbamic acid 1-(4-amino-1-oxobutoxy)ethyl ester trifluoroacetate was added. Then, 50% ethanol aqueous solution of 36 mg (0.08 mmol) of 4-(4,6-dimethoxy-1,3,5-triazin-2-yl)-4-methylmorpholinium chloride (DMT-MM) was rapidly added thereto and stirred at room temperature overnight. 100 μL of a 20% sodium chloride aqueous solution was added to the reaction solution, the reaction solution was added dropwise under stirring to 12 mL of 90% ethanol, and 10 mL of ethanol was added to the mixed liquid and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 200 mg of the title compound. Based on values of integral in ¹H-NMR, the introduction ratio of methoxyphenamine per unit of whole disaccharide (glucuronic acid) of chondroitin sulfate was 5%.

### Reference Example 2

### 2-[[3-oxo-3-[[(troxipide-carbonyl)oxy]methoxy]propyl]amino]-2-oxoethyl-methoxy polyethylene glycol (20,000) conjugate

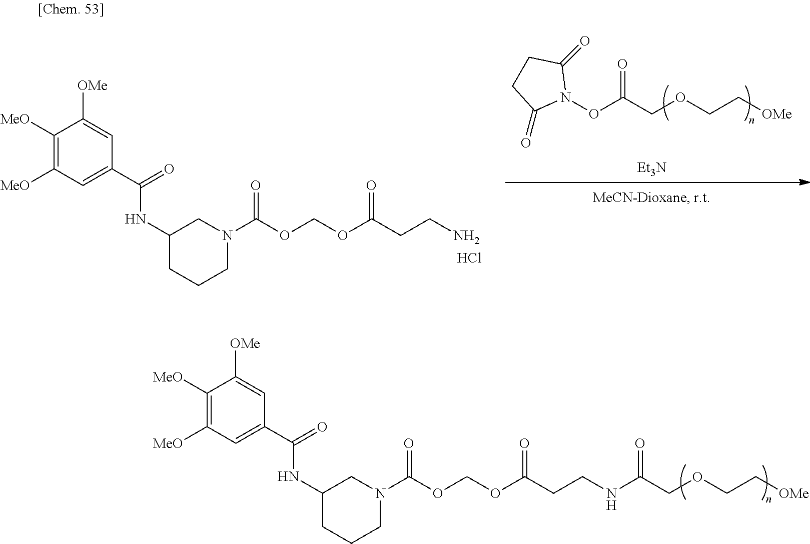

A mixed solution of 35 mg (0.07 mmol) of 3-[(3,4,5-trimethoxybenzoyl)amino]-1-piperidinecarboxylic acid[(3-amino-1-oxopropoxy)]methyl ester hydrochloride in acetonitrile/dioxane (1/1) was added to an acetonitrile solution of 294 mg (0.02 mmol) of methoxy polyethylene glycol (20,000) acetic acid N-hydroxysuccinimide ester and 15 mg (0.15 mmol) of triethylamine and stirred at room temperature overnight. The solvent was distilled off under reduced pressure, the residue was dissolved by adding 1 mL of acetonitrile. Under stirring the solution, isopropyl alcohol was slowly added dropwise until before the reaction solution became cloudy. The suspension was added dropwise to 12 mL of isopropyl alcohol under stirring, and the deposited precipitates were filtered. The precipitates on the filter were washed two times with isopropyl alcohol and one time with diethyl ether. The obtained precipitates were dried at room temperature overnight using a vacuum pump to obtain 285 mg of the title compound. Based on values of integral in ¹H-NMR, the introduction ratio of troxipide was 49.4%.

### Example 32

### 6-[[(1,1-dimethylethoxy)carbonyl]amino]hexanoic acid silver salt

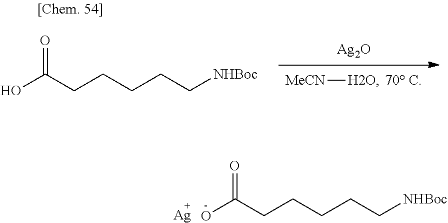

1.39 g (6.0 mmol) of silver oxide (I) was added to a solution of 2.32 g (10.0 mmol) of 6-[[(1,1-dimethylethoxy)carbonyl]amino]hexanoic acid in 20 mL of acetonitrile. Under an argon stream, 10 mL of water was added to the mixed liquid and stirred at 70° C. for 1 hour. The reaction solution was filtered through celite, and the filtrate was condensed under reduced pressure. The residue was dried overnight with a vacuum pump to obtain 2.73 g (8.1 mmol) (81%) of the title compound.

### Example 33

### 6-[[(1,1-dimethylethoxy)carbonyl]amino]hexanoic acid 1-[[(4-nitrophenoxy)carbonyl]oxy]ethyl ester

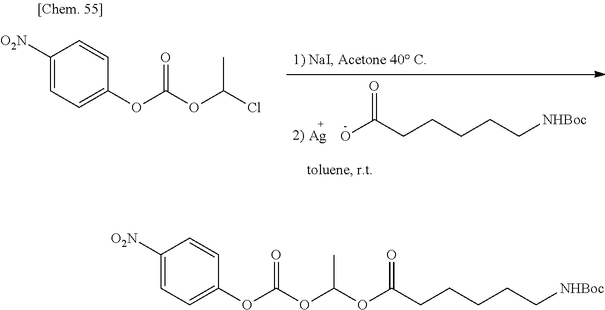

An acetone suspension of 7.12 g (47.5 mmol) of sodium iodide and 2.33 g (9.49 mmol) of carbonic acid 1-chloroethyl 4-nitrophenyl ester was stirred at 40° C. overnight. The reaction solution was filtered, the filtrate was condensed under reduced pressure, and toluene was added thereto and filtered again. The filtrate was condensed, and then a suspension of 2.73 g (8.1 mmol) of 6-[[(1,1-dimethylethoxy)carbonyl]amino]hexanoic acid silver salt in dry toluene was added thereto and stirred at room temperature overnight. Ethyl acetate was added to the reaction solution, the insoluble matter was removed by filtration, and the solvent was distilled off under reduced pressure. The residue was purified by silica gel column chromatography (5% to 30% ethyl acetate/hexane) to obtain 700 mg (1.6 mmol) (17%) of the title compound.

¹H NMR (CDCl₃, δ): 1.31-1.41 (2H, m), 1.44 (9H, s), 1.46-1.53 (2H, m), 1.61 (3H, d, J=5 Hz), 1.67 (2H, quin, J=8 Hz), 2.38 (2H, t, J=8 Hz), 3.05-3.17 (2H, m), 4.51 (1H, br s), 6.84 (1H, q, J=5 Hz), 7.41 (2H, d, J=10 Hz), 8.29 (2H, d, J=9 Hz)

### Example 34

### [3-[1-[(etilefrine-carbonyl)oxy]-2-methylpropoxy]-3-oxo-2-methylpropyl]amino-chondroitin sulfate conjugate

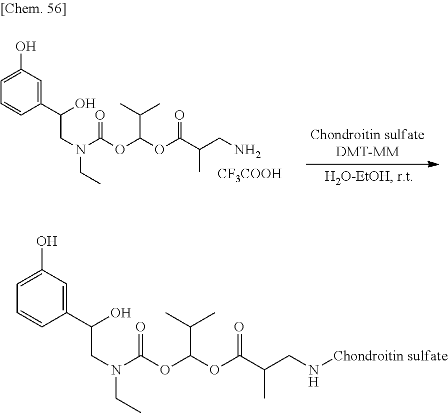

100 mg (0.2 mmol) of sodium chondroitin sulfate was dissolved in 2 mL of water, and 2 mL of ethanol was added dropwise thereto under stirring. To the mixed liquid, 50% ethanol aqueous solution of 19 mg (0.04 mmol) of N-ethyl-N-[2-hydroxy-2-(3-hydroxyphenyl)ethyl]carbamic acid 1-(3-amino-2-methyl-1-oxopropoxy)-2-methylpropyl ester trifluoroacetate was added. Then, 50% ethanol aqueous solution of 18 mg (0.04 mmol) of 4-(4,6-dimethoxy-1,3,5-triazin-2-yl)-4-methylmorpholinium chloride (DMT-MM) was rapidly added thereto and stirred at room temperature overnight. 50 μL of 20% sodium chloride aqueous solution was added to the reaction solution, and ethanol was further added dropwise (about 2 mL) until immediately before the reaction solution became cloudy. The reaction solution was added dropwise to 10 mL of 90% ethanol under stirring and left to stand still. The supernatant was decanted, and then 15 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 89 mg of the title compound. Based on values of integral in ¹H-NMR, the introduction ratio of etilefrine per unit of whole disaccharide (glucuronic acid) of chondroitin sulfate was 17%.

### Example 35

### 3-[(3,4,5-trimethoxybenzoyl)amino]-1-piperidinecarboxylic acid 1-[(3-amino-1-oxopropoxy)]ethyl ester trifluoroacetate

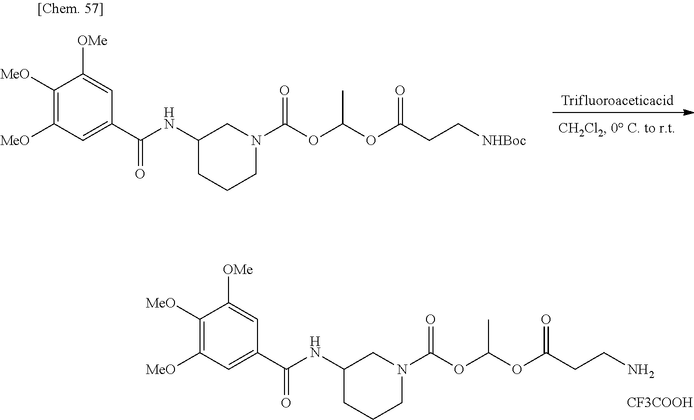 [SMILES: COc1cc(C(=O)NC2CCCN(C(=O)OC(C)OC(=O)CCNC(=O)OC(C)(C)C)C2)cc(OC)c1OC ; "Trifluoroaceticacid" ; COc1cc(C(=O)NC2CCCN(C(=O)OC(C)OC(=O)CCN)C2)cc(OC)c1OC ; "CF3COOH"]

0.1 mL of trifluoroacetic acid was slowly added to under cooling on ice to a methylene chloride solution of 74 mg (0.1 mmol) of 3-[(3,4,5-trimethoxybenzoyl)amino]-1-piperidinecarboxylic acid 1-[3-[[(1,1-dimethylethoxy)carbonyl]amino]-1-oxopropoxy]ethyl ester and stirred at room temperature for 8 hours. The solvent was distilled off under reduced pressure, diisopropyl ether was added to the residue and stirred. The precipitated crystals were filtered to obtain 58 mg (79%) of the title compound.

¹H-NMR (DMSO-d₆, δ): 1.36-1.52 (4H, m), 1.54-1.68 (1H, m), 1.73-1.85 (1H, m), 1.88-1.99 (1H, m), 2.62-2.96 (4H, m), 2.96-3.27 (2H, m), 3.72 (3H, s), 3.76-3.94 (8H, m), 3.99-4.07 (1H, m), 6.66-6.76 (1H, m), 7.16 (2H, s), 7.73 (3H, br s), 8.12 (1H, br d, J=7 Hz)

### Example 36

### [3-oxo-3-[1-[(troxipide-carbonyl)oxy]ethoxy]propyl]amino-chondroitin sulfate conjugate

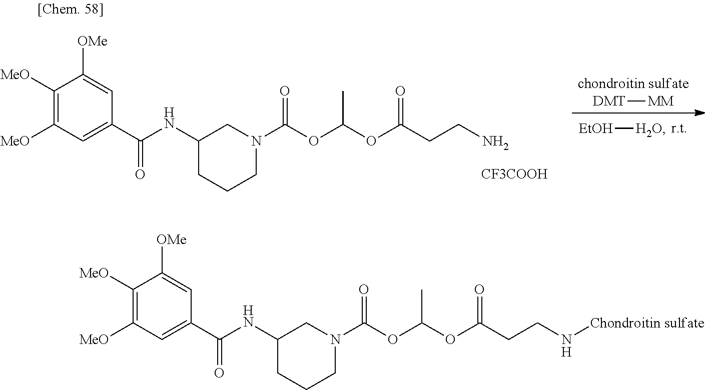

150 mg (0.30 mmol) of sodium chondroitin sulfate was dissolved in 3 mL of water, and 3 mL of ethanol was added dropwise thereto under stirring. To the mixed liquid, 50% of ethanol aqueous solution of 34 mg (0.06 mmol) of 3-[(3,4,5-trimethoxybenzoyl)amino]-1-piperidinecarboxylic acid 1-[(3-amino-1-oxopropoxy)]ethyl ester trifluoroacetate was added. Then, 50% of ethanol aqueous solution of 27 mg (0.06 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 2 mL) until immediately before the reaction solution became cloudy. The reaction solution was added dropwise to 9 mL of ethanol under stirring and left to stand still. The supernatant was decanted, and then 10 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 171 mg of the title compound. Based on values of integral in ¹H-NMR, the introduction ratio of troxipide per unit of whole disaccharide (glucuronic acid) of chondroitin sulfate was 14%.

### Example 37

### 3-[(3,4,5-trimethoxybenzoyl)amino]-1-piperidinecarboxylic acid 1-[(2-aminoacetyl)oxy]ethyl ester trifluoroacetate

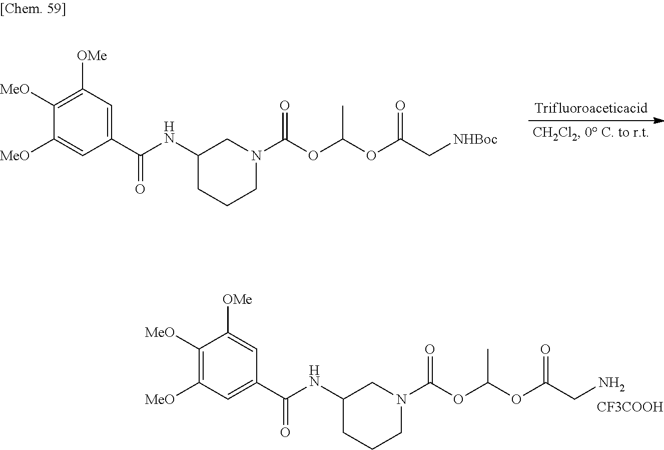 [SMILES: COc1cc(C(=O)NC2CCCN(C(=O)OC(C)OC(=O)CNC(=O)OC(C)(C)C)C2)cc(OC)c1OC ; COc1cc(C(=O)NC2CCCN(C(=O)OC(C)OC(=O)CN)C2)cc(OC)c1OC ; "CF3COOH"]

0.15 mL of trifluoroacetic acid was slowly added under cooling on ice to a methylene chloride solution of 106 mg (0.2 mmol) of 3-[(3,4,5-trimethoxybenzoyl)amino]-1-piperidinecarboxylic acid 1-[[2-[[(1,1-dimethylethoxy)carbonyl]amino]acetyl]oxy]ethyl ester and stirred at room temperature for 5 hours. The solvent was distilled off under reduced pressure, diisopropyl ether was added to the residue and stirred. The precipitated crystals were filtered to obtain 100 mg (90%) of the title compound.

¹H-NMR (DMSO-d₆, δ): 1.39-1.54 (4H, m), 1.55-1.67 (1H, m), 1.73-1.83 (1H, m), 1.86-2.04 (1H, m), 2.72-2.99 (2H, m), 3.72 (3H, s), 3.76-3.97 (10H, m), 4.00-4.10 (1H, m), 6.73-6.85 (1H, m), 7.16 (2H, s), 8.05-8.33 (4H, m)

### Example 38

### [2-[1-[(troxipide-carbonyl)oxy]ethoxy]-2-oxoethyl]amino-chondroitin sulfate conjugate

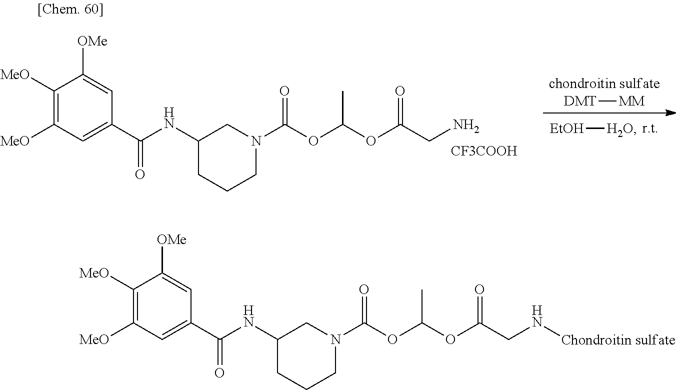

150 mg (0.30 mmol) of sodium chondroitin sulfate was dissolved in 3 mL of water, and 3 mL of ethanol was added dropwise thereto under stirring. To the mixed liquid, 50% ethanol aqueous solution of 33 mg (0.06 mmol) of 3-[(3,4,5-trimethoxybenzoyl)amino]-1-piperidinecarboxylic acid 1-[(2-aminoacetyl)oxy]ethyl ester trifluoroacetate was added. Then, 50% ethanol aqueous solution of 27 mg (0.06 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 2 mL) until immediately before the reaction solution became cloudy. The reaction solution was added dropwise to 9 mL of ethanol under stirring and left to stand still. The supernatant was decanted, and then 10 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 157 mg of the title compound. Based on values of integral in ¹H-NMR, the introduction ratio of troxipide per unit of whole disaccharide (glucuronic acid) of chondroitin sulfate was 18%.

### Example 39

### 3-[(3,4,5-trimethoxybenzoyl)amino]-1-piperidinecarboxylic acid 1-[2-[[(1,1-dimethylethoxy)carbonyl]amino]-2-methyl-1-oxopropoxy]ethyl ester

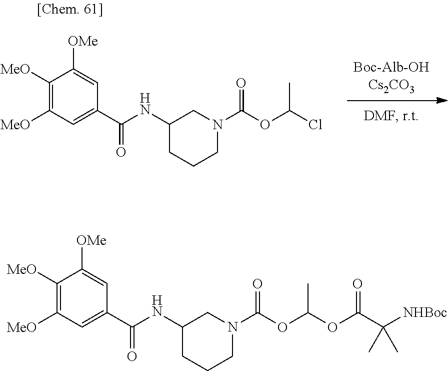 [SMILES: COc1cc(C(=O)NC2CCCN(C(=O)OC(C)Cl)C2)cc(OC)c1OC ; COc1cc(C(=O)NC2CCCN(C(=O)OC(C)OC(=O)C(C)(C)NC(=O)OC(C)(C)C)C2)cc(OC)c1OC]

79 mg (0.2 mmol) of cesium carbonate was added to a methanol solution of 91 mg (0.5 mmol) of 2-[[(1,1-dimethylethoxy)carbonyl]amino]-2-methyl propanoic acid and stirred at room temperature for 10 minutes. The solvent was distilled off under reduced pressure, and dried overnight using a vacuum pump. The obtained 2-[[(1,1-dimethylethoxy)carbonyl]amino]-2-methyl propanoic acid cesium salt was dissolved in N,N-dimethylformamide. An N,N-dimethylformamide solution of 150 mg (0.4 mmol) of 3-[(3,4,5-trimethoxybenzoyl)amino]-1-piperidinecarboxylic acid-1-chloroethyl ester was added to the reaction solution and stirred overnight. Water was added to the reaction solution and extracted three times with diethyl ether. The obtained organic layer was washed with saturated sodium bicarbonate water and saturated saline, and then dried over anhydrous magnesium sulfate, and the solvent was distilled off under reduced pressure. The residue was dried overnight using a vacuum pump to obtain 143 mg (0.3 mmol) (68%) of the title compound.

¹H-NMR (CDCl₃, δ): 1.33-1.54 (18H, m), 1.56-2.12 (4H, m), 2.96-3.71 (3H, m), 3.78-4.07 (10H, m), 4.09-4.34 (1H, m), 4.74-4.96 (1H, m), 6.76 (1H, q, J=5 Hz), 7.02 (2H, s)

### Example 40

### 3-[(3,4,5-trimethoxybenzoyl)amino]-1-piperidinecarboxylic acid 1-(2-amino-2-methyl-1-oxopropoxy)ethyl ester trifluoroacetate

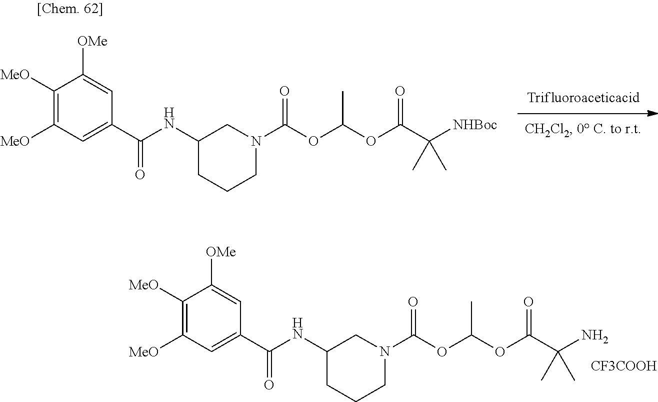 [SMILES: COc1cc(C(=O)NC2CCCN(C(=O)OC(C)OC(=O)C(C)(C)NC(=O)OC(C)(C)C)C2)cc(OC)c1OC ; COc1cc(C(=O)NC2CCCN(C(=O)OC(C)OC(=O)C(C)(C)N)C2)cc(OC)c1OC ; "CF3COOH"]

0.19 mL of trifluoroacetic acid was slowly added under cooling on ice to a methylene chloride solution of 143 mg (0.3 mmol) of 3-[(3,4,5-trimethoxybenzoyl)amino]-1-piperidinecarboxylic acid 1-[2-[[(1,1-dimethylethoxy)carbonyl]amino]-2-methyl-1-oxopropoxy]ethyl ester and stirred at room temperature for 4 hours. The solvent was distilled off under reduced pressure, diisopropyl ether was added to the residue and stirred. The precipitated crystals were filtered to obtain 145 mg (quantitative) of the title compound.

¹H-NMR (DMSO-d₆, δ): 1.32-1.65 (11H, m), 1.71-1.86 (1H, m), 1.87-1.99 (1H, m), 2.67-3.03 (2H, m), 3.70 (3H, s), 3.75-3.94 (8H, m), 3.96-4.10 (1H, m), 6.67-6.81 (1H, m), 7.15 (2H, s), 8.12-8.32 (1H, m), 8.46 (3H, br s)

### Example 41

### [1,1-dimethyl-2-oxo-2-[1-[(troxipide-carbonyl)oxy]ethoxy]ethyl]amino-chondroitin sulfate conjugate

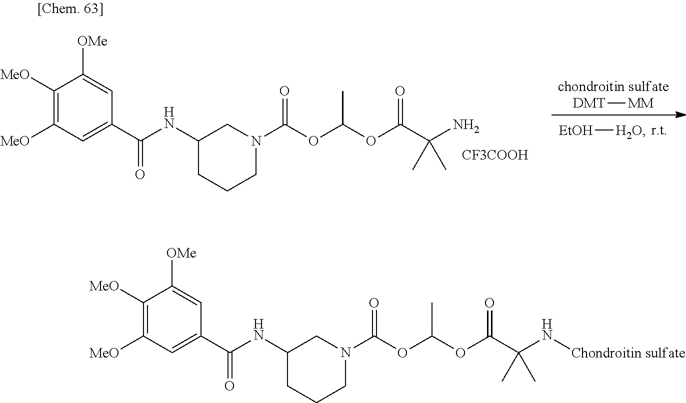

150 mg (0.30 mmol) of sodium chondroitin sulfate was dissolved in 3 mL of water, and 3 mL of ethanol was added dropwise thereto under stirring. To the mixed liquid, 50% ethanol aqueous solution of 34 mg (0.06 mmol) of 3-[(3,4,5-trimethoxybenzoyl)amino]-1-piperidinecarboxylic acid 1-(2-amino-2-methyl-1-oxopropoxy)ethyl ester trifluoroacetate was added. Then, 50% ethanol aqueous solution of 27 mg (0.06 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 2 mL) until immediately before the reaction solution became cloudy. The reaction solution was added dropwise to 9 mL of ethanol under stirring and left to stand still. The supernatant was decanted, and then 10 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 233 mg of the title compound. Based on values of integral in ¹H-NMR, the introduction ratio of troxipide per unit of whole disaccharide (glucuronic acid) of chondroitin sulfate was 2%.

### Example 42

### N-[2-(2-methoxyphenyl)-1-methylethyl]-N-methyl carbamic acid 1-[[[1-[[(1,1-dimethylethoxy)carbonyl]amino]cyclopentyl]carbonyl]oxy]ethyl ester

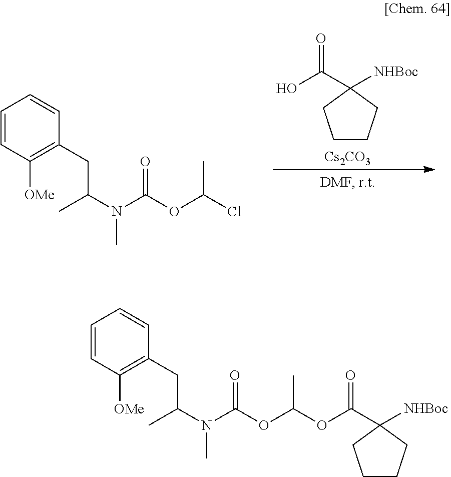 [SMILES: COc1ccccc1CC(C)N(C)C(=O)OC(C)Cl ; "Cs2CO3" ; CC(C)(C)OC(=O)NC1(C(=O)O)CCCC1 ; COc1ccccc1CC(C)N(C)C(=O)OC(C)OC(=O)C1(NC(=O)OC(C)(C)C)CCCC1]

254 mg (0.8 mmol) of cesium carbonate was added to a methanol solution of 325 mg (1.4 mmol) of 1-[[(1,1-dimethylethoxy)carbonyl]amino]cyclopentane carboxylic acid and stirred at room temperature for 10 minutes. The solvent in the mixed suspension was distilled off under reduced pressure, and dried overnight using a vacuum pump. 278 mg (0.8 mmol) was collected from the obtained 1-[[(1,1-dimethylethoxy)carbonyl]amino]cyclopentane carboxylic acid cesium salt and, dissolved in N,N-dimethylformamide, an N,N-dimethylformamide solution of 200 mg (0.7 mmol) of N-[2-(2-methoxyphenyl)-1-methylethyl]-N-methyl carbamic acid 1-chloroethyl ester was added thereto and stirred overnight. Water was added to the reaction solution and extracted with diethyl ether. The organic layer was washed with saturated sodium bicarbonate water and saturated saline, and then dried over anhydrous magnesium sulfate, and the solvent was distilled off under reduced pressure. The residue was dried overnight using a vacuum pump to obtain 259 mg (0.5 mmol) (77%) of the title compound.

¹H-NMR (CDCl₃, δ): 1.09-1.20 (3H, m), 1.23 (1H, d, J=5 Hz), 1.37-1.50 (11H, m), 1.66-1.79 (4H, m), 1.80-2.06 (2H, m), 2.10-2.21 (1H, m), 2.22-2.34 (1H, m), 2.67-2.83 (5H, m), 3.79-3.89 (3H, m), 4.37-4.46 (1/3 H, m), 4.47-4.59 (2/3 H, m), 4.86 (1H, br s), 6.59 (1/3 H, q, J=5 Hz), 6.69-6.80 (2/3 H, m), 6.80-6.91 (2H, m), 7.01-7.12 (1H, m), 7.18 (1H, td, J=8, 2 Hz)

### Example 43

### N-[2-(2-methoxyphenyl)-1-methylethyl]-N-methyl carbamic acid 1-[[(1-aminocyclopentyl)carbonyl]oxy]ethyl ester trifluoroacetate

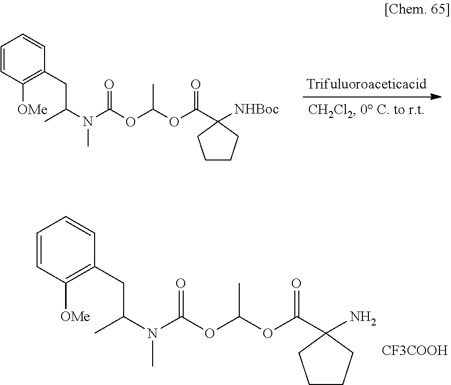 [SMILES: COc1ccccc1CC(C)N(C)C(=O)OC(C)OC(=O)C1(NC(=O)OC(C)(C)C)CCCC1 ; COc1ccccc1CC(C)N(C)C(=O)OC(C)OC(=O)C1(N)CCCC1 ; "CF3COOH"]

0.41 mL of trifluoroacetic acid was slowly added under cooling on ice to a methylene chloride solution of 259 mg (0.5 mmol) of N-[2-(2-methoxyphenyl)-1-methylethyl]-N-methyl carbamic acid 1-[[[1-[[(1,1-dimethylethoxy)carbonyl]amino]cyclopentyl]carbonyl]oxy]ethyl ester and stirred at room temperature for 6 hours. The solvent was distilled off under reduced pressure, hexane was added to the residue and stirred. The supernatant was decanted and dried with a vacuum pump overnight to obtain 226 mg of the title compound.

¹H-NMR (DMSO-d₆, δ): 1.06-1.16 (3H, m), 1.22 (1H, d, J=5 Hz), 1.37-1.49 (2H, m), 1.57-1.95 (6H, m), 1.99-2.21 (2H, m), 2.65-2.76 (5H, m), 3.71-3.85 (3H, m), 4.31-4.49 (1H, m), 6.41 (1/3 H, q, J=5 Hz), 6.57-6.69 (2/3 H, m), 6.79-6.88 (1H, m), 6.91-6.97 (1H, m), 6.99-7.12 (1H, m), 7.13-7.32 (1H, m), 8.45 (3H, br s)

### Example 44

### [1-[[1-[(methoxyphenamine-carbonyl)oxy]ethoxy]carbonyl]cyclopentyl]amino-chondroitin sulfate conjugate

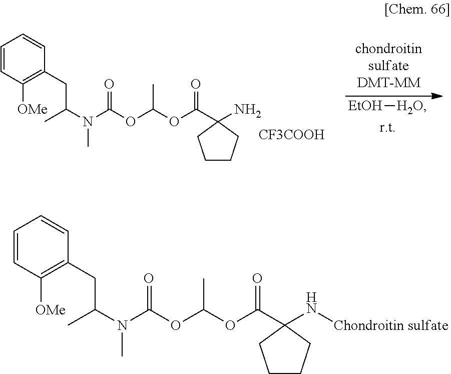

150 mg (0.30 mmol) of sodium chondroitin sulfate was dissolved in 3 mL of water, and 3 mL of ethanol was added dropwise thereto under stirring. To the mixed liquid, 50% ethanol aqueous solution of 29 mg (0.06 mmol) of N-[2-(2-methoxyphenyl)-1-methylethyl]-N-methyl carbamic acid 1-[[(1-aminocyclopentyl)carbonyl]oxy]ethyl ester trifluoroacetate was added. Then, 50% ethanol aqueous solution of 27 mg (0.06 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 2 mL) until immediately before the reaction solution became cloudy. The reaction solution was added dropwise to 9 mL of ethanol under stirring and left to stand still. The supernatant was decanted, and then 10 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 221 mg of the title compound. Based on values of integral in ¹H-NMR, the introduction ratio of methoxyphenamine per unit of whole disaccharide (glucuronic acid) of chondroitin sulfate was 1%.

### Example 45

### N-[2-(2-methoxyphenyl)-1-methylethyl]-N-methyl carbamic acid 1-[[[1-[[(1,1-dimethylethoxy)carbonyl]amino]cyclopropyl]carbonyl]oxy]ethyl ester

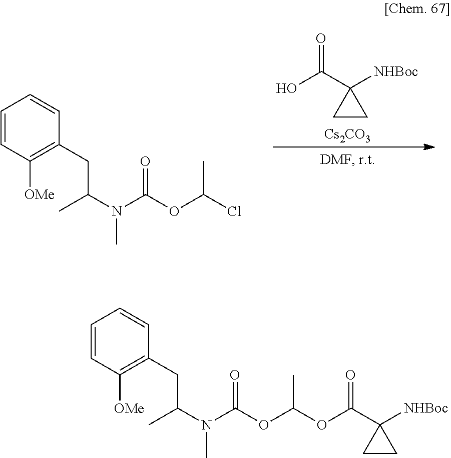

312 mg (1.0 mmol) of cesium carbonate was added to a methanol solution of 350 mg (1.7 mmol) of 1-[[(1,1-dimethylethoxy)carbonyl]amino]cyclopropane carboxylic acid and stirred at room temperature for 10 minutes. The solvent in the mixed suspension was distilled off under reduced pressure, and dried overnight using a vacuum pump. 257 mg (0.8 mmol) was collected from the obtained 1-[[(1,1-dimethylethoxy)carbonyl]amino]cyclopropane carboxylic acid cesium salt and, dissolved in N, N-dimethylformamide, and an N,N-dimethylformamide solution of 200 mg (0.7 mmol) of N-[2-(2-methoxyphenyl)-1-methylethyl]-N-methyl carbamic acid 1-chloroethyl ester ester was added thereto and stirred overnight. Water was added to the reaction solution and extracted with diethyl ether. The organic layer was washed with saturated sodium bicarbonate water and saturated saline, and then dried over anhydrous magnesium sulfate, and the solvent was distilled off under reduced pressure. The residue was dried overnight using a vacuum pump to obtain 148 mg (0.3 mmol) (47%) of the title compound.

¹H-NMR (CDCl₃, δ): 1.07-1.23 (6H, m), 1.35-1.54 (13H, m), 2.67-2.85 (5H, m), 3.80-3.85 (3H, m), 4.34-4.47 (1/2 H, m), 4.49-4.60 (1/2 H, m), 5.08 (1H, br s), 6.53-6.61 (1/3 H, m), 6.65-6.78 (2/3 H, m), 6.80-6.92 (2H, m), 6.99-7.14 (1H, m), 7.14-7.23 (1H, m)

### Example 46

### N-[2-(2-methoxyphenyl)-1-methylethyl]-N-methyl carbamic acid 1-[[(1-aminocyclopropyl)carbonyl]oxy]ethyl ester trifluoroacetate

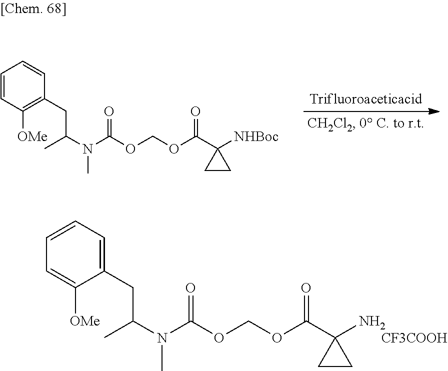 [SMILES: COc1ccccc1CC(C)N(C)C(=O)OCOC(=O)C1(NC(=O)OC(C)(C)C)CC1 ; COc1ccccc1CC(C)N(C)C(=O)OCOC(=O)C1(N)CC1 ; "CF3COOH"]

0.25 mL of trifluoroacetic acid was slowly added under cooling on ice to a methylene chloride solution of 148 mg (0.3 mmol) of N-[2-(2-methoxyphenyl)-1-methylethyl]-N-methyl carbamic acid 1-[[[1-[[(1,1-dimethylethoxy)carbonyl]amino]cyclopropyl]carbonyl]oxy]ethyl ester and stirred at room temperature for 6 hours. The solvent was distilled off under reduced pressure, hexane was added to the residue and stirred. The supernatant was decanted and dried with a vacuum pump overnight to obtain 144 mg of the title compound.

¹H-NMR (DMSO-d₆, δ): 1.06-1.19 (4H, m), 1.22-1.49 (6H, m), 2.65-2.75 (5H, m), 3.75-3.82 (3H, m), 4.31-4.48 (1H, m), 6.39 (1/3 H, q, J=5 Hz), 6.52-6.60 (2/3 H, m), 6.81-6.88 (1H, m), 6.90-6.98 (1H, m), 7.00-7.10 (1H, m), 7.14-7.32 (1H, m), 8.68 (3H, br s)

### Example 47

### [1-[[1-[(methoxyphenamine-carbonyl)oxy]ethoxy]carbonyl]cyclopropyl]amino-chondroitin sulfate conjugate

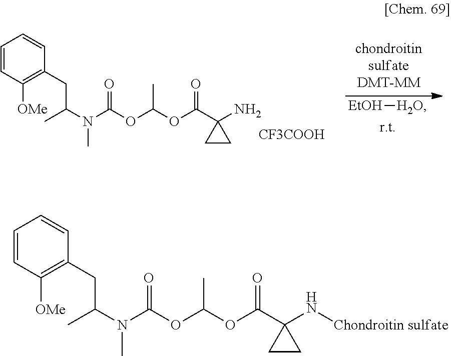

150 mg (0.30 mmol) of sodium chondroitin sulfate was dissolved in 3 mL of water, and 3 mL of ethanol was added dropwise thereto under stirring. To the mixed liquid, 50% ethanol aqueous solution of 27 mg (0.06 mmol)N-[2-(2-methoxyphenyl)-1-methylethyl]-N-methyl carbamic acid 1-[[(1-aminocyclopropyl)carbonyl]oxy]ethyl ester trifluoroacetate was added. Then, 50% ethanol aqueous solution of 27 mg (0.06 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 2 mL) until immediately before the reaction solution became cloudy. The reaction solution was added dropwise to 9 mL of ethanol under stirring and left to stand still. The supernatant was decanted, and then 10 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 145 mg of the title compound. Based on values of integral in ¹H-NMR, the introduction ratio of methoxyphenamine per unit of whole disaccharide (glucuronic acid) of chondroitin sulfate was 10%.

### Example 48

### N-methyl-N-[4-(trifluoromethyl)phenyl]carbamic acid chloromethyl ester

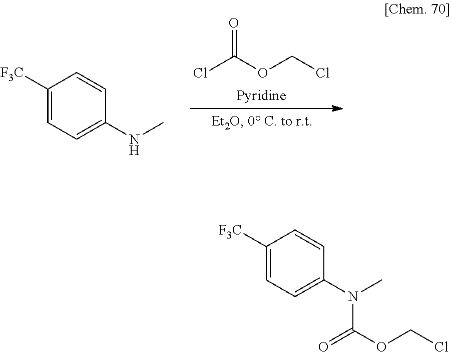 [SMILES: CNc1ccc(C(F)(F)F)cc1 ; O=C(Cl)OCCl ; CN(C(=O)OCCl)c1ccc(C(F)(F)F)cc1]

A diethyl ether solution of 496 mg (3.9 mmol) of chloromethyl chloroformate was added under cooling on ice to a diethyl ether solution of 613 mg (3.5 mmol) of N-methyl-4-(trifluoromethyl)aniline and 305 mg (3.9 mmol) of pyridine and stirred at room temperature for 1 hour. The reaction solution was filtered, and the solvent was distilled off under reduced pressure to obtain 926 mg (3.5 mmol) (quantitative) of the title compound.

¹H-NMR (CDCl₃, δ): 3.38 (3H, s), 5.78 (2H, s), 7.39 (2H, br d, J=8 Hz), 7.64 (2H, d, J=9 Hz)

### Example 49

### N-methyl-N-[4-(trifluoromethyl)phenyl]carbamic acid[3-[[(1,1-dimethylethoxy)carbonyl]amino]-1-oxobutoxy]methyl ester

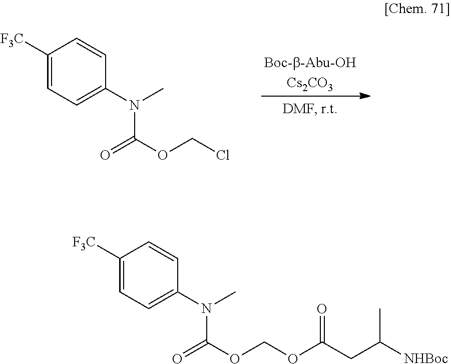 [SMILES: CN(C(=O)OCCl)c1ccc(C(F)(F)F)cc1 ; "Cs2CO3" ; CC(CC(=O)OCOC(=O)N(C)c1ccc(C(F)(F)F)cc1)NC(=O)OC(C)(C)C]

408 mg (1.3 mmol) of cesium carbonate was added to a methanol solution of 509 mg (2.5 mmol) of 3-[[(1,1-dimethylethoxy)carbonyl]amino]butyric acid and stirred at room temperature for 30 minutes. The solvent in the mixed suspension was distilled off under reduced pressure, and dried overnight using a vacuum pump. 275 mg (0.8 mmol) was collected from the obtained 3-[[(1,1-dimethylethoxy)carbonyl]amino]butyric acid cesium salt and, dissolved in N,N-dimethylformamide, an N,N-dimethylformamide solution of 200 mg (0.8 mmol) of N-methyl-N-[4-(trifluoromethyl)phenyl]carbamic acid chloromethyl ester was added thereto and stirred overnight. Water was added to the reaction solution and extracted with diethyl ether. The organic layer was washed with saturated sodium bicarbonate water and saturated saline, and then dried over anhydrous magnesium sulfate, and the solvent was distilled off under reduced pressure. The residue was dried overnight using a vacuum pump to obtain 275 mg (0.6 mmol) (84%) of the title compound.

1H-NMR (CDCl3, δ): 1.21 (3H, d, J=7 Hz), 1.43 (9H, s), 2.48-2.66 (2H, m), 3.36 (3H, s), 3.96-4.16 (1H, m), 4.82 (1H, br s), 5.79 (2H, s), 7.32-7.47 (2H, m), 7.62 (2H, d, J=9 Hz)

### Example 50

### N-methyl-N-[4-(trifluoromethyl)phenyl]carbamic acid (3-amino-1-oxobutoxy)methyl ester hydrochloride

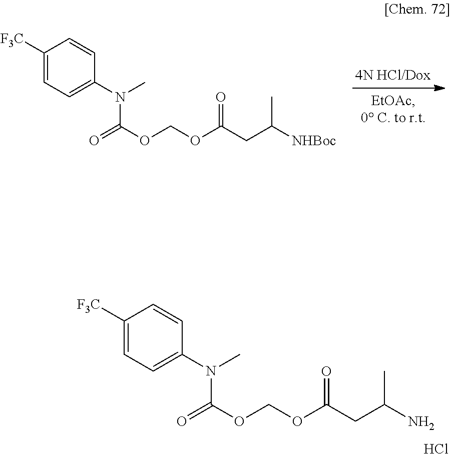 [SMILES: CC(CC(=O)OCOC(=O)N(C)c1ccc(C(F)(F)F)cc1)NC(=O)OC(C)(C)C ; CC(N)CC(=O)OCOC(=O)N(C)c1ccc(C(F)(F)F)cc1 ; Cl]

1.6 mL of 4 N hydrochloric acid/dioxane solution was added to an ethyl acetate solution of 275 mg (0.6 mmol) of N-methyl-N-[4-(trifluoromethyl)phenyl]carbamic acid[3-[[(1,1-dimethylethoxy)carbonyl]amino]-1-oxobutoxy]methyl ester, and the reaction solution was left to stand still for 3 hours. The solvent was distilled off under reduced pressure, diisopropyl ether was added to the residue and stirred. The precipitated crystals were filtered to obtain 253 mg (quantitative) of the title compound. 1H-NMR (DMSO-d₆, δ): 1.22 (3H, d, J=7 Hz), 2.67 (1H, dd, J=17, 8 Hz), 2.81 (1H, dd, J=17, 6 Hz), 3.30 (3H, s), 3.47-3.56 (1H, m), 5.71-5.77 (2H, m), 7.57 (2H, d, J=8 Hz), 7.76 (2H, d, J=8 Hz), 8.02 (3H, br s)

### Example 51

### [1-methyl-3-[[[[methyl[4-(trifluoromethyl)phenyl]amino]carbonyl]oxy]methoxy]-3-oxopropyl]amino-chondroitin sulfate conjugate

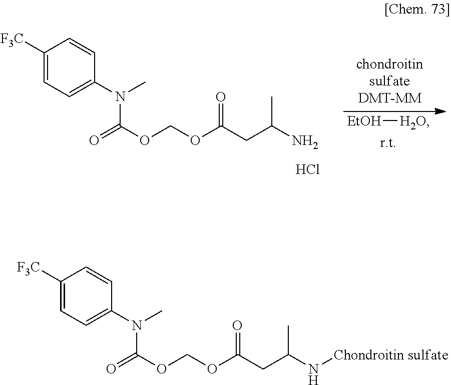

150 mg (0.30 mmol) of sodium chondroitin sulfate was dissolved in 3 mL of water, and 3 mL of ethanol was added dropwise thereto under stirring. To the mixed liquid, 50% ethanol aqueous solution of 22 mg (0.06 mmol) of N-methyl-N-[4-(trifluoromethyl)phenyl]carbamic acid (3-amino-1-oxobutoxy)methyl ester hydrochloride was added. Then, 50% ethanol aqueous solution of 27 mg (0.06 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 2 mL) until immediately before the reaction solution became cloudy. The reaction solution was added dropwise to 9 mL of ethanol under stirring and left to stand still. The supernatant was decanted, and then 10 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 153 mg of the title compound. Based on values of integral in ¹H-NMR, the introduction ratio of N-methyl-4-(trifluoromethyl)aniline per unit of whole disaccharide (glucuronic acid) of chondroitin sulfate was 10%.

### Example 52

### N-methyl-N-[4-(trifluoromethyl)phenyl]carbamic acid[(2S)-2-[[(1,1-dimethylethoxy)carbonyl]amino]-1-oxopropoxy]methyl ester

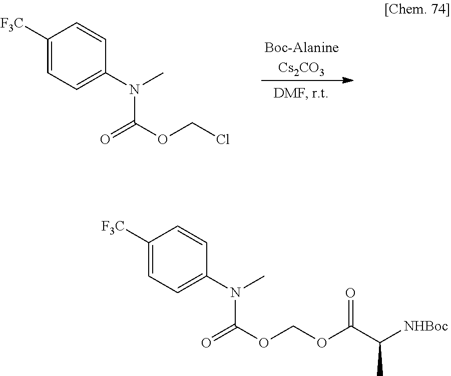 [SMILES: CN(C(=O)OCCl)c1ccc(C(F)(F)F)cc1 ; "Boc-AlanineCs2CO3" ; C[C@H](NC(=O)OC(C)(C)C)C(=O)OCOC(=O)N(C)c1ccc(C(F)(F)F)cc1]

73 mg (0.2 mmol) of cesium carbonate was added to a methanol solution of 78 mg (0.4 mmol) of N-[(1,1-dimethylethoxy)carbonyl]-L-alanine and stirred at room temperature for 10 minutes. The solvent in the mixed suspension was distilled off under reduced pressure. The obtained N-[(1,1-dimethylethoxy)carbonyl]-L-alanine cesium salt was dissolved in N,N-dimethylformamide, and an N,N-dimethylformamide solution of of 100 mg (0.4 mmol) of N-methyl-N-[4-(trifluoromethyl)phenyl]carbamic acid chloromethyl ester was added thereto and stirred overnight. Water was added to the reaction solution and extracted with diethyl ether. The organic layer was washed with saturated sodium bicarbonate water and saturated saline, and then dried over anhydrous magnesium sulfate, and the solvent was distilled off under reduced pressure. The residue was dried overnight using a vacuum pump to obtain 150 mg (0.4 mmol) (quantitative) of the title compound. 1H-NMR (CDCl₃, δ): 1.40 (3H, d, J=7 Hz), 1.44 (9H, s), 3.36 (3H, s), 4.27-4.41 (1H, m), 4.97 (1H, br s), 5.76-5.89 (2H, m), 7.32-7.47 (2H, m), 7.62 (2H, d, J=8 Hz)

### Example 53

### N-methyl-N-[4-(trifluoromethyl)phenyl]carbamic acid[(2S)-2-amino-1-oxopropoxy]methyl ester hydrochloride

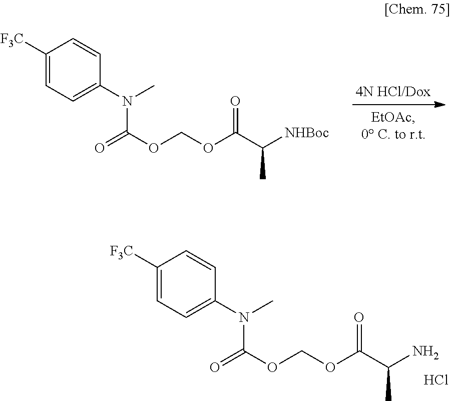 [SMILES: C[C@H](NC(=O)OC(C)(C)C)C(=O)OCOC(=O)N(C)c1ccc(C(F)(F)F)cc1 ; C[C@H](N)C(=O)OCOC(=O)N(C)c1ccc(C(F)(F)F)cc1 ; Cl]

1.5 mL of 4 N hydrochloric acid/dioxane solution was added to an ethyl acetate solution of 147 mg (0.3 mmol) of N-methyl-N-[4-(trifluoromethyl)phenyl]carbamic acid[(2S)-2-[[(1,1-dimethylethoxy)carbonyl]amino]-1-oxopropoxy]methyl ester, and the reaction solution was left to stand still for 8 hours. The solvent was distilled off under reduced pressure, diisopropyl ether was added to the residue and stirred. The precipitated crystals were filtered to obtain 96 mg (0.3 mmol) (79%) of the title compound.

1H-NMR (DMSO-d₆, δ): 1.41 (3H, d, J=7 Hz), 3.31 (3H, s), 4.18 (1H, q, J=7 Hz), 5.77-5.88 (2H, m), 7.58 (2H, d, J=8 Hz), 7.77 (2H, d, J=8 Hz), 8.43 (3H, br s)

### Example 54

### [(1S)-1-methyl-2-[[[[methyl[4-(trifluoromethyl)phenyl]amino]carbonyl]oxy]methoxy]-2-oxoethyl]amino-chondroitin sulfate conjugate

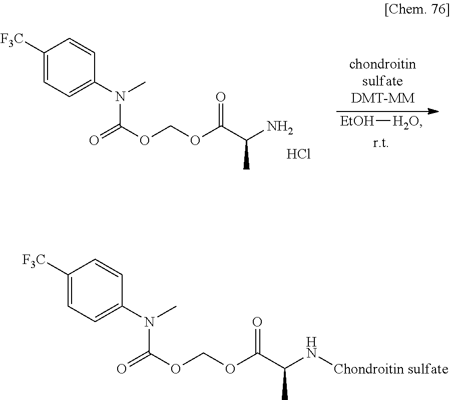

150 mg (0.30 mmol) of sodium chondroitin sulfate was dissolved in 3 mL of water, and 3 mL of ethanol was added dropwise thereto under stirring. To the mixed liquid, 50% ethanol aqueous solution of 21 mg (0.06 mmol) of N-methyl-N-[4-(trifluoromethyl)phenyl]carbamic acid[(2S)-2-amino-1-oxopropoxy]methyl ester hydrochloride was added. Then, 50% ethanol aqueous solution of 27 mg (0.06 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 2 mL) until immediately before the reaction solution became cloudy. The reaction solution was added dropwise to 9 mL of ethanol under stirring and left to stand still. The supernatant was decanted, and then 10 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 148 mg of the title compound. Based on values of integral in ¹H-NMR, the introduction ratio of N-methyl-4-(trifluoromethyl)aniline per unit of whole disaccharide (glucuronic acid) of chondroitin sulfate was 15%.

### Example 55

### N-methyl-N-[4-(trifluoromethyl)phenyl]carbamic acid[(2S)-2-[[(1,1-dimethylethoxy)carbonyl]amino]-3-methyl-1-oxobutoxy]methyl ester

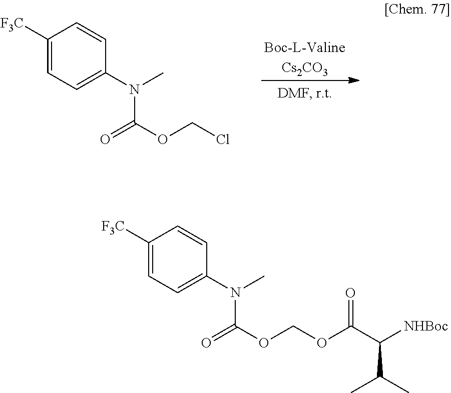 [SMILES: CN(C(=O)OCCl)c1ccc(C(F)(F)F)cc1 ; "Boc-L-ValineCs2CO3" ; CC(C)[C@H](NC(=O)OC(C)(C)C)C(=O)OCOC(=O)N(C)c1ccc(C(F)(F)F)cc1]

73 mg (0.2 mmol) of cesium carbonate was added to a methanol solution of 89 mg (0.4 mmol) of N-[(1,1-dimethylethoxy)carbonyl]-L-valine and stirred at room temperature for 10 minutes. The solvent in the mixed suspension was distilled off under reduced pressure. The obtained N-[(1,1-dimethylethoxy)carbonyl]-L-valine cesium salt was dissolved in N,N-dimethylformamide, and an N,N-dimethylformamide solution of 100 mg (0.4 mmol) of N-methyl-N-[4-(trifluoromethyl)phenyl]carbamic acid chloromethyl ester was added thereto and stirred overnight. Water was added to the reaction solution and extracted with diethyl ether. The organic layer was washed with saturated sodium bicarbonate water and saturated saline, and then dried over anhydrous magnesium sulfate, and the solvent was distilled off under reduced pressure. The residue was dried overnight using a vacuum pump to obtain 130 mg (0.3 mmol) (78%) of the title compound.

1H-NMR (CDCl₃, δ): 0.86 (3H, d, J=7 Hz), 0.95 (3H, d, J=7 Hz), 1.44 (9H, s), 2.07-2.22 (1H, m), 3.35 (3H, s), 4.26 (1H, br dd, J=8, 4 Hz), 4.96 (1H, br d, J=8 Hz), 5.70-5.94 (2H, m), 7.32-7.42 (2H, m), 7.62 (2H, d, J=9 Hz)

### Example 56

### N-methyl-N-[4-(trifluoromethyl)phenyl]carbamic acid[(2S)-2-amino-3-methyl-1-oxobutoxy]methyl ester hydrochloride

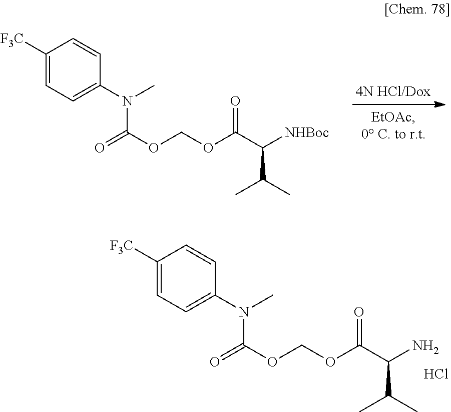 [SMILES: CC(C)[C@H](NC(=O)OC(C)(C)C)C(=O)OCOC(=O)N(C)c1ccc(C(F)(F)F)cc1 ; CC(C)[C@H](N)C(=O)OCOC(=O)N(C)c1ccc(C(F)(F)F)cc1 ; Cl]

2 mL of 4 N hydrochloric acid/dioxane solution was added to an ethyl acetate solution of 126 mg (0.3 mmol) of N-methyl-N-[4-(trifluoromethyl)phenyl]carbamic acid[(2S)-2-[[(1,1-dimethylethoxy)carbonyl]amino]-3-methyl-1-oxobutoxy]methyl ester, and the reaction solution was left to stand still for 5 hours. The solvent was distilled off under reduced pressure, diisopropyl ether was added to the residue and stirred. The precipitated crystals were filtered to obtain 103 mg (0.3 mmol) (quantitative) of the title compound.

1H-NMR (DMSO-d₆, δ): 0.92 (3H, d, J=7 Hz), 0.96 (3H, d, J=7 Hz), 2.11-2.23 (1H, m), 3.30 (3H, s), 4.02 (1H, br d, J=4 Hz), 5.74-5.95 (2H, m), 7.56 (2H, d, J=9 Hz), 7.77 (2H, d, J=9 Hz), 8.44 (3H, br s)

### Example 57

### [(1S)-2-methyl-1-[[[[methyl[4-(trifluoromethyl)phenyl]amino]carbonyl]oxy]methoxy]carbonyl]propyl]amino-chondroitin sulfate conjugate

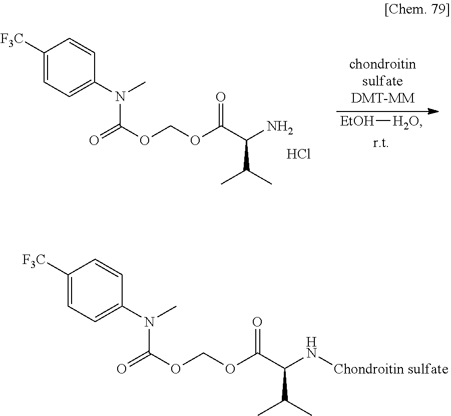

150 mg (0.30 mmol) of sodium chondroitin sulfate was dissolved in 3 mL of water, and 3 mL of ethanol was added dropwise thereto under stirring. To the mixed liquid, 50% ethanol aqueous solution of 23 mg (0.06 mmol) of N-methyl-N-[4-(trifluoromethyl)phenyl]carbamic acid[(2S)-2-amino-3-methyl-1-oxobutoxy]methyl ester hydrochloride was added. Then, 50% ethanol aqueous solution of 27 mg (0.06 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 2 mL) until immediately before the reaction solution became cloudy. The reaction solution was added dropwise to 9 mL of ethanol under stirring and left to stand still. The supernatant was decanted, and then 10 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 147 mg of the title compound. Based on values of integral in ¹H-NMR, the introduction ratio of N-methyl-4-trifluoromethylaniline per unit of whole disaccharide (glucuronic acid) of chondroitin sulfate was 16%.

### Example 58

### N-methyl-N-[4-(trifluoromethyl)phenyl]carbamic acid[(2S)-3,3-dimethyl-2-[[(1,1-dimethylethoxy)carbonyl]amino]-1-oxobutoxy]methyl ester

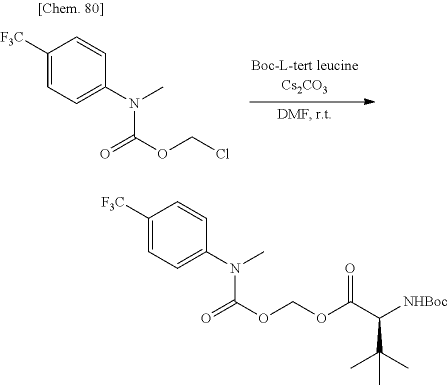 [SMILES: CN(C(=O)OCCl)c1ccc(C(F)(F)F)cc1 ; CN(C(=O)OCOC(=O)[C@@H](NC(=O)OC(C)(C)C)C(C)(C)C)c1ccc(C(F)(F)F)cc1]

73 mg (0.2 mmol) of cesium carbonate was added to a methanol solution of 95 mg (0.4 mmol) of N-[(1,1-dimethylethoxy)carbonyl]-L-tert-leucine and stirred at room temperature for 10 minutes. The solvent in the mixed suspension was distilled off under reduced pressure. The obtained N-[(1,1-dimethylethoxy)carbonyl]-L-tert-leucine cesium salt was dissolved in N,N-dimethylformamide, and an N,N-dimethylformamide solution of 100 mg (0.4 mmol) of N-methyl-N-[4-(trifluoromethyl)phenyl]carbamic acid chloromethyl ester was added thereto and stirred overnight. Water was added to the reaction solution and extracted with diethyl ether. The organic layer was washed with saturated sodium bicarbonate water and saturated saline, and then dried over anhydrous magnesium sulfate, and the solvent was distilled off under reduced pressure. The residue was dried overnight using a vacuum pump to obtain 100 mg (0.2 mmol) (58%) of the title compound.

1H-NMR (CDCl₃, δ): 0.95 (9H, s), 1.43 (9H, s), 3.35 (3H, s), 4.01-4.23 (1H, m), 5.05 (1H, br d, J=8 Hz), 5.72-5.92 (2H, m), 7.36 (2H, br d, J=8 Hz), 7.61 (2H, d, J=8 Hz)

### Example 59

### N-methyl-N-[4-(trifluoromethyl)phenyl]carbamic acid[(2S)-2-amino-3,3-dimethyl-1-oxobutoxy]methyl ester hydrochloride

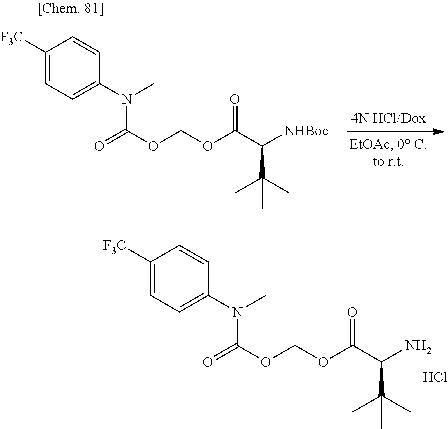 [SMILES: CN(C(=O)OCOC(=O)[C@@H](NC(=O)OC(C)(C)C)C(C)(C)C)c1ccc(C(F)(F)F)cc1 ; CN(C(=O)OCOC(=O)[C@@H](N)C(C)(C)C)c1ccc(C(F)(F)F)cc1 ; Cl]

1.5 mL of 4 N hydrochloric acid/dioxane solution was added to an ethyl acetate solution of 94 mg (0.2 mmol) of N-methyl-N-[4-(trifluoromethyl)phenyl]carbamic acid[(2S)-3,3-dimethyl-2-[[(1,1-dimethylethoxy)carbonyl]amino]-1-oxobutoxy]methyl ester, and the reaction solution was left to stand still for 5 hours. The solvent was distilled off under reduced pressure, diisopropyl ether was added to the residue and stirred. The precipitated crystals were filtered to obtain 77 mg (0.2 mmol) (quantitative) of the title compound.

1H-NMR (DMSO-d₆, δ): 0.97 (9H, s), 3.30 (3H, s), 3.82 (1H, s), 5.70-5.97 (2H, m), 7.56 (2H, d, J=9 Hz), 7.76 (2H, d, J=9 Hz), 7.90-8.43 (3H, br s)

### Example 60

### [(1S)-2,2-dimethyl-1-[[[[[methyl[4-(trifluoromethyl)phenyl]amino]carbonyl]oxy]methoxy]carbonyl]propyl]amino-chondroitin sulfate conjugate

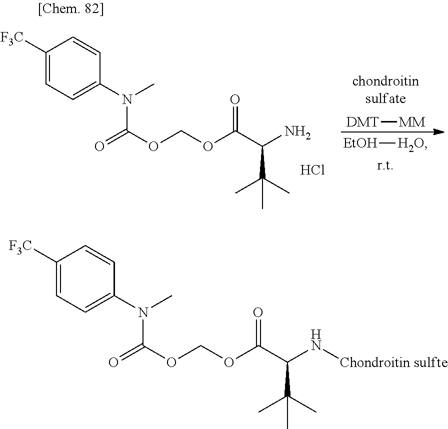

150 mg (0.30 mmol) of sodium chondroitin sulfate was dissolved in 3 mL of water, and 3 mL of ethanol was added dropwise thereto under stirring. To the mixed liquid, 50% ethanol aqueous solution of 24 mg (0.06 mmol) of N-methyl-N-[4-(trifluoromethyl)phenyl]carbamic acid[(2S)-2-amino-3,3-dimethyl-1-oxobutoxy]methyl ester hydrochloride was added. Then, 50% ethanol aqueous solution of 27 mg (0.06 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 3 mL) until immediately before the reaction solution became cloudy. The reaction solution was added dropwise to 9 mL of ethanol under stirring and left to stand still. The supernatant was decanted, and then 10 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 151 mg of the title compound. Based on values of integral in ¹H-NMR, the introduction ratio of N-methyl-4-(trifluoromethyl)aniline per unit of whole disaccharide (glucuronic acid) of chondroitin sulfate was 20%.

### Example 61

### [2-(2,6-dimethylphenoxy)-1-methylethyl]carbamic acid 1-[6-[[(1,1-dimethylethoxy)carbonyl]amino]-1-oxohexyloxy]ethyl ester

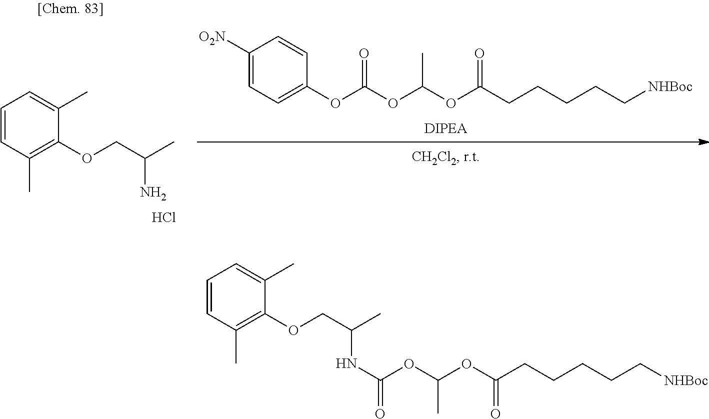 [SMILES: Cc1cccc(C)c1OCC(C)N ; CC(OC(=O)CCCCCNC(=O)OC(C)(C)C)OC(=O)Oc1ccc([N+](=O)[O-])cc1 ; Cl ; Cc1cccc(C)c1OCC(C)NC(=O)OC(C)OC(=O)CCCCCNC(=O)OC(C)(C)C]

A methylene chloride solution of 121 mg (0.27 mmol) of 6-[[(1,1-dimethylethoxy)carbonyl]amino]hexanoic acid 1-[[(4-nitrophenoxy)carbonyl]oxy]ethyl ester was added to a methylene chloride solution of 59 mg (0.27 mmol) of mexiletine hydrochloride and 71 mg (0.55 mmol) of diisopropylethylamine and stirred at room temperature overnight. The solvent was distilled off under reduced pressure, and the residue was purified by silica gel column chromatography (10% to 35% ethyl acetate/hexane) to obtain 56 mg (0.12 mmol) (44%) of the title compound.

1H-NMR (CDCl₃, δ): 1.30-1.38 (2H, m), 1.39-1.51 (17H, m), 1.59-1.71 (2H, m), 2.26 (6H, s), 2.27-2.41 (2H, m), 3.10 (2H, br s), 3.67-3.75 (1H, m), 3.75-3.87 (1H, m), 4.06 (1H, br s), 4.56 (1H, br s), 5.18 (1H, br s), 6.81-6.88 (1H, m), 6.90-6.95 (1H, m), 6.98-7.02 (2H, m)

### Example 62

### [2-(2,6-dimethylphenoxy)-1-methylethyl]carbamic acid 1-(6-amino-1-oxohexyloxy)ethyl ester trifluoroacetate

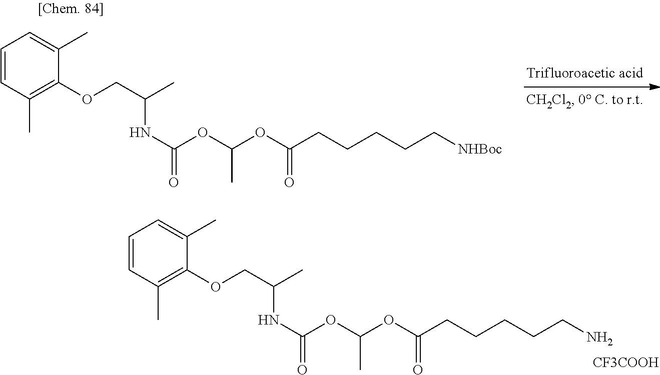

0.1 mL of trifluoroacetic acid was slowly added under cooling on ice to a methylene chloride solution of 56 mg (0.12 mmol) of [[2-(2,6-dimethylphenoxy)]-1-methylethyl]carbamic acid 1-[6-[[(1,1-dimethylethoxy)carbonyl]amino]-1-oxohexyloxy]ethyl ester and stirred at room temperature for 6 hours. The solvent was distilled off under reduced pressure, diisopropyl ether was added to the residue and stirred. The supernatant was decanted and dried with a vacuum pump overnight to obtain 37 mg of the title compound.

¹H-NMR (DMSO-d₆, δ): 1.18-1.24 (3H, m), 1.25-1.36 (2H, m), 1.40 (3H, t, J=5.7 Hz), 1.45-1.58 (4H, m), 2.20 (6H, s), 2.23-2.34 (2H, m), 2.70-2.81 (2H, m), 3.61-3.66 (2H, m), 3.80-3.91 (1H, m), 6.67-6.73 (1H, m), 6.90 (1H, t, J=8 Hz), 7.00 (2H, d, J=8 Hz), 7.51-7.73 (4H, m)

### Example 63

### [6-[1-[(mexiletine-carbonyl)oxy]ethoxy]-6-oxohexyl]amino-chondroitin sulfate conjugate

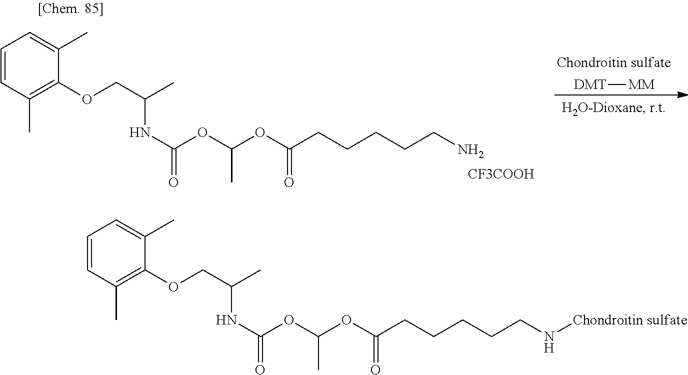

100 mg (0.2 mmol) of sodium chondroitin sulfate was dissolved in 2 mL of water, and 2 mL of dioxane was added dropwise thereto under stirring. To the mixed liquid, 50% dioxane aqueous solution of 19 mg (0.04 mmol) of [[2-(2,6-dimethylphenoxy)]-1-methylethyl]carbamic acid 1-(6-amino-1-oxohexyloxy)ethyl ester trifluoroacetate was added. Then, 50% ethanol aqueous solution of 18 mg (0.04 mmol) of 4-(4,6-dimethoxy-1,3,5-triazin-2-yl)-4-methylmorpholinium chloride (DMT-MM) was rapidly added thereto and stirred at room temperature overnight. 50 μL of 20% sodium chloride aqueous solution was added to the reaction solution, and ethanol was further added dropwise (about 2 mL) until immediately before the reaction solution became cloudy. The reaction solution was added dropwise to 10 mL of 90% ethanol under stirring and left to stand still. The supernatant was decanted, and then 15 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 104 mg of the title compound. Based on values of integral in ¹H-NMR, the introduction ratio of mexiletine per unit of whole disaccharide (glucuronic acid) of chondroitin sulfate was 14%.

### Example 64

### [2-(2,6-dimethylphenoxy)-1-methylethyl]carbamic acid[3-[[(1,1-dimethylethoxy)carbonyl]amino]-1-oxopropoxy]methyl ester

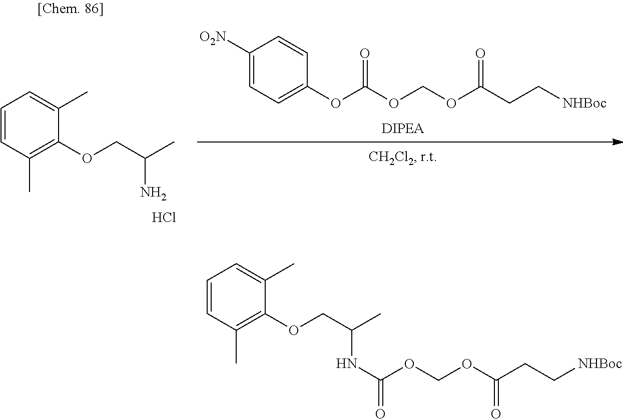 [SMILES: Cc1cccc(C)c1OCC(C)N ; CC(C)(C)OC(=O)NCCC(=O)OCOC(=O)Oc1ccc([N+](=O)[O-])cc1 ; Cl ; Cc1cccc(C)c1OCC(C)NC(=O)OCOC(=O)CCNC(=O)OC(C)(C)C]

A methylene chloride solution of 175 mg (0.45 mmol) of N-[(1,1-dimethylethoxy)carbonyl]-β-alanine[[(4-nitrophenoxy)carbonyl]oxy]methyl ester was added to a methylene chloride solution of 98 mg (0.45 mmol) of mexiletine hydrochloride and 117 mg (0.91 mmol) of diisopropylethylamine and stirred at room temperature overnight. The solvent was distilled off under reduced pressure and the residue was purified by silica gel column chromatography (10% to 50% ethyl acetate/hexane). The obtained crude product was purified again by silica gel column chromatography (10% ethyl acetate/toluene) to obtain 149 mg (0.35 mmol) (78%) of the title compound.

1H-NMR (CDCl₃, δ): 1.39-1.47 (12H, m), 2.25 (6H, s), 2.59 (2H, t, J=6 Hz), 3.34-3.47 (2H, m), 3.73 (1H, dd, J=9, 4 Hz), 3.80 (1H, dd, J=9, 4 Hz), 4.02-4.18 (1H, m), 5.01 (1H, br s), 5.26 (1H, br d, J=8 Hz), 5.77 (2H, s), 6.90-6.95 (1H, m), 7.00 (2H, d, J=8 Hz)

### Example 65

### [2-(2,6-dimethylphenoxy)-1-methylethyl]carbamic acid(3-amino-1-oxopropoxy)methyl ester hydrochloride

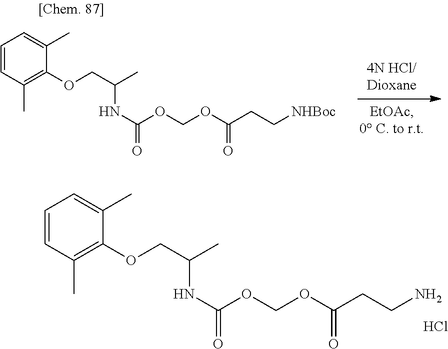 [SMILES: Cc1cccc(C)c1OCC(C)NC(=O)OCOC(=O)CCNC(=O)OC(C)(C)C ; Cc1cccc(C)c1OCC(C)NC(=O)OCOC(=O)CCN ; Cl]

4 mL of 4 N hydrochloric acid/dioxane solution was added to an ethyl acetate solution of 149 mg (0.35 mmol) of [[2-(2,6-dimethylphenoxy)]-1-methylethyl]carbamic acid[3-[[(1,1-dimethylethoxy)carbonyl]amino]-1-oxopropoxy]methyl ester, and the reaction solution was left to stand still for 6 hours. The solvent was distilled off under reduced pressure, diisopropyl ether was added to the residue and stirred. The supernatant was decanted and dried with a vacuum pump overnight to obtain 112 mg (0.31 mmol) (89%) of the title compound.

¹H-NMR (DMSO-d₆, δ): 1.23 (3H, d, J=7 Hz), 2.20 (6H, s), 2.74 (2H, t, J=7 Hz), 3.02 (2H, t, J=7 Hz), 3.64 (2H, d, J=6 Hz), 3.84-3.95 (1H, m), 5.69 (2H, s), 6.87-6.94 (1H, m), 7.01 (2H, d, J=7 Hz), 7.72 (1H, d, J=8 Hz), 8.00 (3H, br s)

### Example 66

### [3-[[(mexiletine-carbonyl)oxy]methoxy]-3-oxopropyl]amino-chondroitin sulfate conjugate

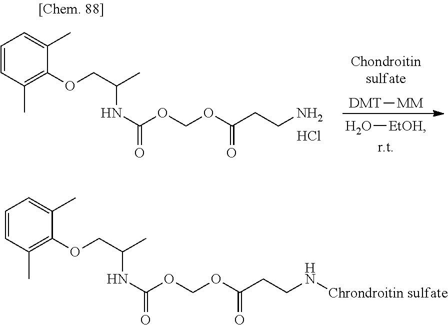

200 mg (0.4 mmol) of sodium chondroitin sulfate was dissolved in 4 mL of water, and 4 mL of ethanol was added dropwise thereto under stirring. To the mixed liquid, 50% ethanol aqueous solution of 28 mg (0.08 mmol) of [[1-methyl-2-(2,6-dimethylphenoxy)]ethyl]carbamic acid(3-amino-1-oxopropoxy)methyl ester hydrochloride was added. Then, 50% ethanol aqueous solution of 22 mg (0.05 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 3 mL) until immediately before the reaction solution became cloudy. The reaction solution was added dropwise to 11 mL of 90% ethanol under stirring and left to stand still. The supernatant was decanted, and then 15 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight with a vacuum pump to obtain 207 mg of the title compound. Based on values of integral in ¹H-NMR, the introduction ratio of mexiletine per unit of whole disaccharide (glucuronic acid) of chondroitin sulfate was 11%.

### Example 67

### [2-[[(E)-[5-methoxy-1-[4-(trifluoromethyl)phenyl]pentylidene]amino]oxy]ethyl]carbamic acid 1-[6-[[(1,1-dimethylethoxy)carbonyl]amino]-1-oxohexyloxy]ethyl ester

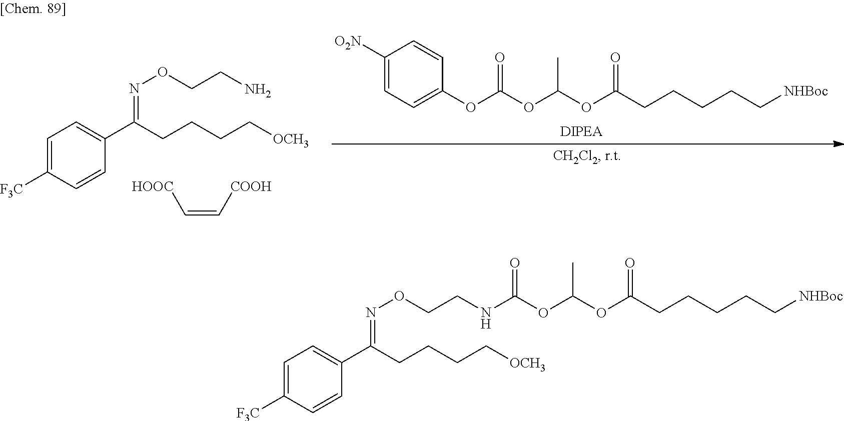 [SMILES: COCCCC/C(=N\OCCN)c1ccc(C(F)(F)F)cc1 ; O=C(O)/C=C\C(=O)O ; CC(OC(=O)CCCCCNC(=O)OC(C)(C)C)OC(=O)Oc1ccc([N+](=O)[O-])cc1 ; COCCCC/C(=N\OCCNC(=O)OC(C)OC(=O)CCCCCNC(=O)OC(C)(C)C)c1ccc(C(F)(F)F)cc1]

A methylene chloride solution of 101 mg (0.23 mmol) of 6-[[(1,1-dimethylethoxy)carbonyl]amino]hexanoic acid 1-[[(4-nitrophenoxy)carbonyl]oxy]ethyl ester was added to a methylene chloride solution of 200 mg (0.46 mmol) of fluvoxamine maleate and 89 mg (0.69 mmol) of diisopropylethylamine and stirred at room temperature overnight. The solvent was distilled off under reduced pressure, and the residue was purified by silica gel column chromatography (10% to 50% ethyl acetate/hexane) to obtain 134 mg (0.19 mmol, purity of 90 mol %) (84%) of the title compound.

1H-NMR (CDCl₃, δ): 1.21-1.41 (2H, m), 1.41-1.50 (14H, m), 1.58-1.69 (6H, m), 2.25-2.35 (2H, m), 2.79 (2H, t, J=7 Hz), 3.02-3.17 (2H, m), 3.32 (3H, s), 3.35-3.44 (2H, m), 3.47-3.66 (2H, m), 4.27 (2H, t, J=5 Hz), 4.58 (1H, br s), 5.37 (1H, br s), 6.83 (1H, q, J=5 Hz), 7.62 (2H, d, J=8 Hz), 7.74 (2H, d, J=8 Hz)

### Example 68

### [2-[[(E)-[5-methoxy-1-[4-(trifluoromethyl)phenyl]pentylidene]amino]oxy]ethyl]carbamic acid 1-(6-amino-1-oxohexyloxy)ethyl ester trifluoroacetate

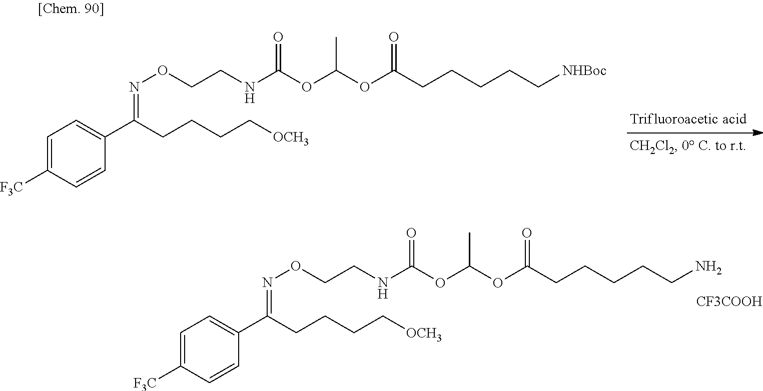 [SMILES: COCCCC/C(=N\OCCNC(=O)OC(C)OC(=O)CCCCCNC(=O)OC(C)(C)C)c1ccc(C(F)(F)F)cc1 ; COCCCC/C(=N\OCCNC(=O)OC(C)OC(=O)CCCCCN)c1ccc(C(F)(F)F)cc1 ; "CF3COOH"]

0.15 mL of trifluoroacetic acid was slowly added under cooling on ice to a methylene chloride solution of 133 mg (0.19 mmol, purity of 90 mol %) of [2-[[(E)-[5-methoxy-1-[4-(trifluoromethyl)phenyl]pentylidene]amino]oxy]ethyl]carbamic acid 1-[6-[[(1,1-dimethylethoxy)carbonyl]amino]-1-oxohexyloxy]ethyl ester and stirred at room temperature for 5 hours. The solvent was distilled off under reduced pressure and the residue was dissolved in toluene. Hexane was added thereto and the precipitates were deposited. The supernatant was decanted and dried with a vacuum pump overnight to obtain 123 mg (quantitative) of the title compound.

¹H-NMR (DMSO-d6, δ): 1.22-1.35 (2H, m), 1.37 (3H, d, J=5 Hz), 1.43-1.55 (8H, m), 2.27 (2H, t, J=7 Hz), 2.71-2.81 (4H, m), 3.19 (3H, s), 3.23-3.41 (4H, m), 4.15 (2H, t, J=6 Hz), 6.67 (1H, q, J=5 Hz), 7.54 (1H, t, J=6 Hz), 7.61 (3H, br s), 7.77 (2H, d, J=8 Hz), 7.86 (2H, d, J=8 Hz)

### Example 69

### [6-[1-[(fluvoxamine-carbonyl)oxy]ethoxy]-6-oxohexyl]amino-chondroitin sulfate conjugate

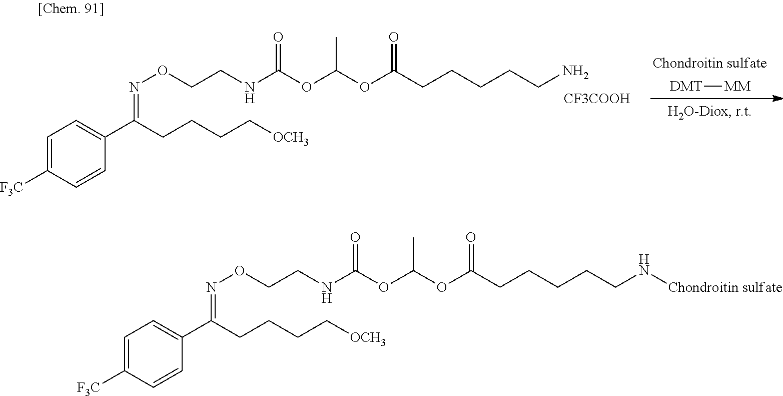

100 mg (0.2 mmol) of sodium chondroitin sulfate was dissolved in 2 mL of water, and 2 mL of dioxane was added dropwise thereto under stirring. To the mixed liquid, 50% ethanol aqueous solution of 25 mg (0.04 mmol) of [2-[[(E)-[5-methoxy-1-[4-(trifluoromethyl)phenyl]pentylidene]amino]oxy]ethyl]carbamic acid 1-(6-amino-1-oxohexyloxy)ethyl ester trifluoroacetate was added. Then, 50% aqueous ethanol of 18 mg (0.04 mmol) of 4-(4,6-dimethoxy-1,3,5-triazin-2-yl)-4-methylmorpholinium chloride (DMT-MM) was rapidly added thereto and stirred at room temperature overnight. 50 μL of 20% sodium chloride aqueous solution was added to the reaction solution, and ethanol was further added dropwise (about 2 mL) until immediately before the reaction solution became cloudy. The reaction solution was added dropwise to 10 mL of 90% ethanol under stirring and left to stand still. The supernatant was decanted, and then 15 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 103 mg of the title compound. Based on values of integral in ¹H-NMR, the introduction ratio of fluvoxamine per unit of whole disaccharide (glucuronic acid) of chondroitin sulfate was 11%.

### Example 70

### [2-[[(E)-[5-methoxy-1-[4-(trifluoromethyl)phenyl]pentylidene]amino]oxy]ethyl]carbamic acid 1-chloro-2-methylpropyl ester

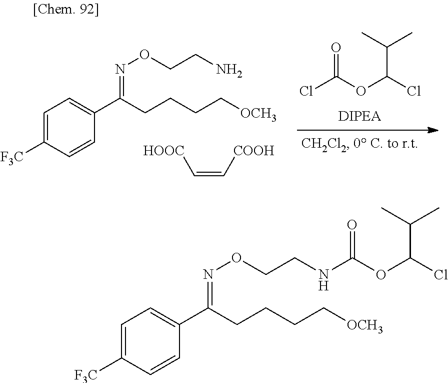

A methylene chloride solution of 189 mg (1.11 mmol) of 1-chloro-2-methylpropyl chloroformate was added under cooling on ice to a methylene chloride solution of 300 mg (0.69 mmol) of fluvoxamine maleate and 223 mg (1.73 mmol) of diisopropylethylamine and stirred at room temperature for 4 hours. The reaction solution was condensed, and then the residue was dissolved in ethyl acetate and washed with a 10% potassium hydrogen sulfate aqueous solution, saturated sodium bicarbonate water, and saturated saline. After drying with anhydrous magnesium sulfate, the solvent was distilled off under reduced pressure and the residue was purified by silica gel column chromatography (10% ethyl acetate/hexane). The solvent was distilled off under reduced pressure, and the residue was dried overnight using a vacuum pump to obtain 140 mg (0.31 mmol) (45%) of the title compound.

¹H-NMR (CDCl₃, δ): 1.04 (3H, d, J=6 Hz), 1.05 (3H, d, J=6 Hz), 1.60-1.66 (4H, m), 2.09-2.20 (1H, m), 2.79 (2H, t, J=8 Hz), 3.33 (3H, s), 3.40 (2H, t, J=6 Hz), 3.58 (2H, dd, J=10, 5 Hz), 4.26-4.33 (2H, m), 5.53 (1H, br s), 6.34 (1H, d, J=4 Hz), 7.62 (2H, d, J=8 Hz), 7.74 (2H, d, J=8 Hz)

### Example 71

### [2-[[(E)-[5-methoxy-1-[4-(trifluoromethyl)phenyl]pentylidene]amino]oxy]ethyl]carbamic acid 1-[(2S)-2-[[(1,1-dimethylethoxy)carbonyl]amino]-1-oxo-2-phenylethoxy]-2-methylpropyl ester

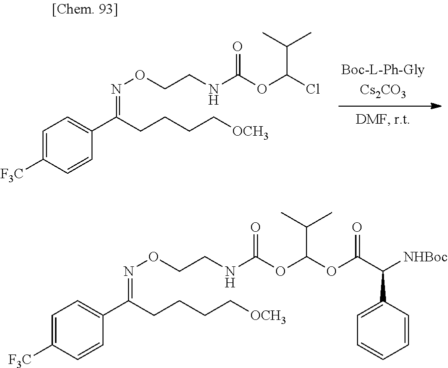 [SMILES: COCCCC/C(=N\OCCNC(=O)OC(Cl)C(C)C)c1ccc(C(F)(F)F)cc1 ; "Boc-L-Ph-GlyCs2CO3" ; COCCCC/C(=N\OCCNC(=O)OC(OC(=O)[C@@H](NC(=O)OC(C)(C)C)c1ccccc1)C(C)C)c1ccc(C(F)(F)F)cc1]

358 mg (1.1 mmol) of cesium carbonate was added to a methanol solution of 502 mg (2.0 mmol) of N-[(1,1-dimethylethoxy)carbonyl]-L-2-phenylglycine and stirred at room temperature for 30 minutes. The solvent in the mixed suspension was distilled off under reduced pressure, and dried overnight using a vacuum pump. 366 mg (0.95 mmol) was collected from the obtained N-[(1,1-dimethylethoxy)carbonyl]-L-2-phenylglycine cesium salt, and dissolved in N,N-dimethylformamide, an N,N-dimethylformamide solution of 140 mg (0.31 mmol) of [2-[[(E)-[5-methoxy-1-[4-(trifluoromethyl)phenyl]pentylidene]amino]oxy]ethyl]carbamic acid 1-chloro-2-methylpropyl ester was added thereto and stirred overnight. Water was added to the reaction solution and extracted with diethyl ether. The organic layer was washed with saturated sodium bicarbonate water and saturated saline, and then dried over anhydrous magnesium sulfate, and the solvent was distilled off under reduced pressure. The residue was purified by silica gel column chromatography (20% ethyl acetate/hexane). The solvent was distilled off under reduced pressure, and the residue was dried overnight using a vacuum pump to obtain 71 mg (0.11 mmol) (34%) of the title compound.

1H-NMR (CDCl₃, δ): 0.63-0.75 (3H, m), 0.94 (3H, d, J=7 Hz), 1.22-1.49 (9H, m), 1.56-1.68 (4H, m), 1.78-1.90 (1/2 H, m), 1.95-2.10 (1/2 H, m), 2.71-2.86 (2H, m), 3.26 (1H, s), 3.32 (2H, s), 3.35-3.45 (3H, m), 3.48-3.64 (1H, m), 4.07-4.23 (1H, m), 4.29 (1H, t, J=5 Hz), 5.17-5.25 (1/2 H, m), 5.28-5.36 (1H, m), 5.36-5.45 (1/2H, m), 5.47-5.59 (1/2 H, m), 5.67-5.77 (1/2 H, m), 6.58-6.69 (1H, m), 7.28-7.39 (5H, m), 7.59-7.63 (2H, m), 7.69-7.77 (2H, m)

### Example 72

### [2-[[(E)-[5-methoxy-1-[4-(trifluoromethyl)phenyl]pentylidene]amino]oxy]ethyl]carbamic acid 1-[(2S)-2-amino-1-oxo-2-phenylethoxy]-2-methylpropyl ester trifluoroacetate

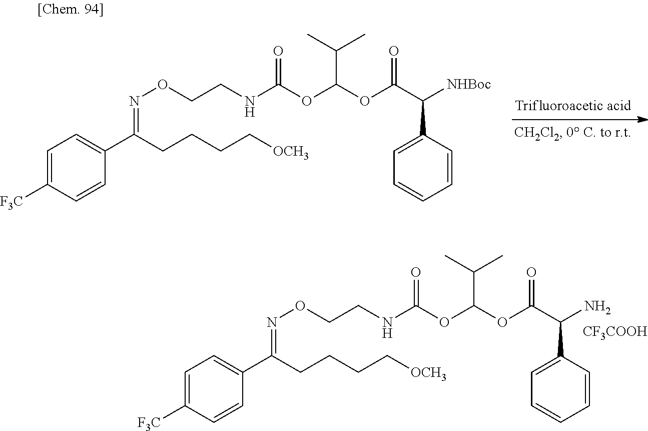 [SMILES: COCCCC/C(=N\OCCNC(=O)OC(OC(=O)[C@@H](NC(=O)OC(C)(C)C)c1ccccc1)C(C)C)c1ccc(C(F)(F)F)cc1 ; COCCCC/C(=N\OCCNC(=O)OC(OC(=O)[C@@H](N)c1ccccc1)C(C)C)c1ccc(C(F)(F)F)cc1 ; "CF3COOH"]

0.25 mL of trifluoroacetic acid was slowly added under cooling on ice to a solution of 69 mg (0.10 mmol) of [2-[[(E)-[5-methoxy-1-[4-(trifluoromethyl)phenyl]pentylidene]amino]oxy]ethyl]carbamic acid 1-[(2S)-2-[[(1,1-dimethylethoxy)carbonyl]amino]-1-oxo-2-phenylethoxy]-2-methylpropyl ester in 1 mL of methylene chloride and stirred at room temperature for 3 hours. The solvent was distilled off under reduced pressure, hexane and diisopropyl ether were added to the residue, and the mixture was stirred. The supernatant was decanted and dried with a vacuum pump overnight to obtain 64 mg (0.09 mmol) (95%) of the title compound.

¹H-NMR (DMSO-d₆, δ): 0.57 (3/2 H, d, J=7 Hz), 0.59 (3/2 H, d, J=7 Hz), 0.86-0.92 (3H, m), 1.41-1.54 (4H, m), 1.71-1.80 (1/2 H, m), 1.90-1.98 (1/2 H, m), 2.71-2.82 (2H, m), 3.18 (3H, s), 3.23-3.32 (4H, m), 3.37-3.45 (1H, m), 3.98-4.12 (1H, m), 4.12-4.22 (1H, m), 5.35-5.42 (1H, m), 6.57 (1/2 H, d, J=5 Hz), 6.59 (1/2 H, d, J=5 Hz), 7.39-7.54 (5H, m), 7.77 (2H, dd, J=9, 2 Hz), 7.86 (2H, d, J=9 Hz), 8.78-8.99 (3H, m)

### Example 73

### [[2-[1-[(fluvoxamine-carbonyl)oxy]-2-methylpropoxy]-2-oxo-1-phenyl]ethyl]amino-chondroitin sulfate conjugate

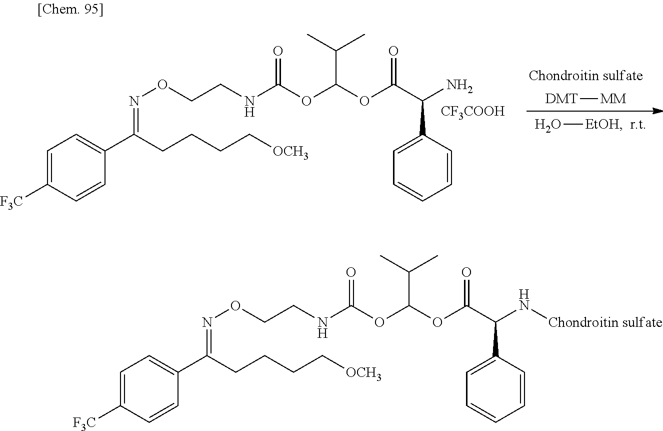

200 mg (0.4 mmol) of sodium chondroitin sulfate was dissolved in 4 mL of water, and 4 mL of ethanol was added dropwise thereto under stirring. To the mixed liquid, 50% ethanol aqueous solution of 53 mg (0.08 mmol) of [2-[[(E)-[5-methoxy-1-[4-(trifluoromethyl)phenyl]pentylidene]amino]oxy]ethyl]carbamic acid 1-[(2S)-2-amino-1-oxo-2-phenylethoxy]-2-methylpropyl ester trifluoroacetate was added. Then, 50% ethanol aqueous solution of 22 mg (0.05 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 4 mL) until immediately before the reaction solution became cloudy. The reaction solution was added dropwise to 12 mL of 90% ethanol under stirring and left to stand still. The supernatant was decanted, and then 15 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 226 mg of the title compound. Based on values of integral in ¹H-NMR, the introduction ratio of fluvoxamine per unit of whole disaccharide (glucuronic acid) of chondroitin sulfate was 11%.

### Example 74

### [4-(3-ethyl-2,6-dioxo-3-piperidinyl)phenyl]carbamic acid chloromethyl ester

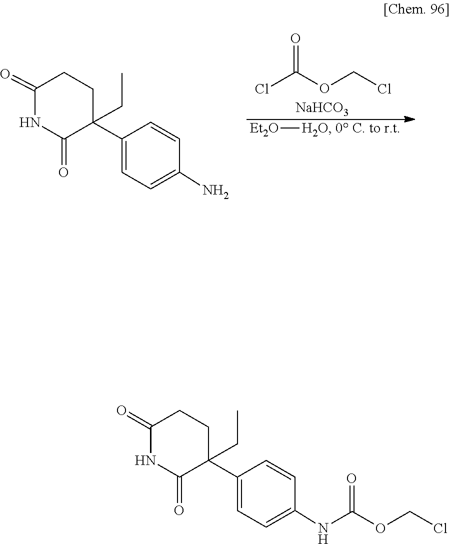

To a mixed solution of 2000 mg (8.6 mmol) of DL-aminoglutethimide and 2170 mg (25.8 mmol) of sodium hydrogen carbonate in diethyl ether-water (3 mL: 3 mL), a diethyl ether solution of 1221 mg (9.47 mmol) of chloromethyl chloroformate was added under cooling on ice, and the resultant solution was stirred at room temperature for 30 minutes. The aqueous layer was removed, ethyl acetate was added, and the organic layer was washed with water and saturated saline. The residue was dried over anhydrous magnesium sulfate, the solvent was distilled off under reduced pressure, and the residue was dried overnight using a vacuum pump to obtain 2514 mg (7.7 mmol) (90%) of the title compound.

¹H-NMR (CDCl₃, δ): 0.87 (3H, t, J=7 Hz), 1.91 (1H, dq, J=14, 7 Hz), 2.04 (1H, dq, J=14, 7 Hz), 2.22 (1H, td, J=14, 5 Hz), 2.32-2.46 (2H, m), 2.56-2.64 (1H, m), 5.83 (2H, s), 6.87 (1H, br s), 7.25 (2H, d, J=9 Hz), 7.43 (2H, br d, J=8 Hz), 7.91 (1H, s)

### Example 75

### [4-(3-ethyl-2,6-dioxo-3-piperidinyl)phenyl]carbamic acid iodomethyl ester

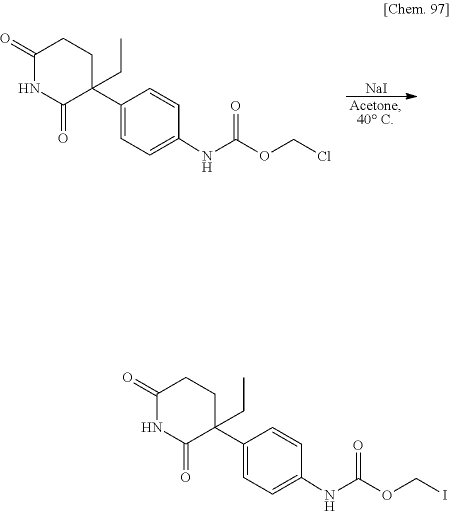 [SMILES: CCC1(c2ccc(NC(=O)OCCl)cc2)CCC(=O)NC1=O ; "NaI" ; "Acetone,40° C." ; CCC1(c2ccc(NC(=O)OCI)cc2)CCC(=O)NC1=O]

An acetone suspension of 296 mg (2.0 mmol) of sodium iodide and 475 mg (1.5 mmol) of [4-(3-ethyl-2,6-dioxo-3-piperidinyl)phenyl]carbamic acid chloromethyl ester was stirred at 40° C. overnight. The reaction solution was condensed under reduced pressure, and diethyl ether was added to the residue and stirred. The insoluble matter was removed by filtration, and the solvent was distilled off under reduced pressure to obtain 615 mg (quantitative) of the title compound. The obtained compound was used for the next step without purification.

### Example 76

### [4-[3-ethyl-2,6-dioxo-3-piperidinyl)phenyl]carbamic acid[2-[[(1,1-dimethylethoxy)carbonyl]amino]-2-methyl-1-oxopropoxy]methyl ester

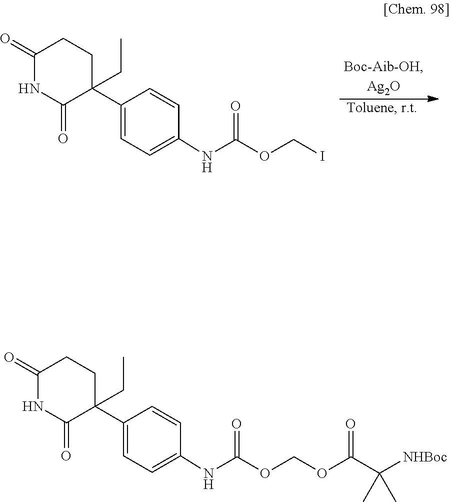 [SMILES: CCC1(c2ccc(NC(=O)OCI)cc2)CCC(=O)NC1=O ; "Toluene, r.t." ; "Boc-Aib-OH,Ag2O" ; CCC1(c2ccc(NC(=O)OCOC(=O)C(C)(C)NC(=O)OC(C)(C)C)cc2)CCC(=O)NC1=O]

383 mg (1.7 mmol) of silver oxide (I) was added to a solution of 610 mg (3.0 mmol) of 2-[[(1,1-dimethylethoxy)carbonyl]amino]-2-methyl propanoic acid in 10 mL of acetonitrile. Under an argon stream, 5 mL of water was added to the mixed liquid and stirred at 70° C. for 2 hours. The reaction solution was filtered through celite, and the filtrate was condensed under reduced pressure. The residue was dried overnight with a vacuum pump to obtain 932 mg of 2-[[(1,1-dimethylethoxy)carbonyl]amino]-2-methyl propanoic acid silver salt. To a suspension of 223 mg (0.72 mmol) of the obtained 2-[[(1,1-dimethylethoxy)carbonyl]amino]-2-methyl propanoic acid silver salt in dry toluene, a solution of 298 mg (0.72 mmol) of [4-(3-ethyl-2,6-dioxo-3-piperidinyl)phenyl]carbamic acid iodomethyl ester in dry toluene was added. The resultant solution was stirred at room temperature overnight. Ethyl acetate was added to the reaction solution, the insoluble matter was removed by filtration, and the solvent was distilled off under reduced pressure. The residue was purified by silica gel column chromatography (15% to 80% ethyl acetate/hexane) to obtain 91 mg (0.18 mmol) (26%) of the title compound.

¹H-NMR (CDCl₃, 5): 0.86 (3H, t, J=8 Hz), 1.37 (9H, s), 1.46 (3H, s), 1.50 (3H, s), 1.90 (1H, dq, J=15, 7 Hz), 1.97-2.07 (1H, m), 2.17-2.26 (1H, m), 2.32-2.45 (2H, m), 2.55-2.64 (1H, m), 4.91 (1H, br s), 5.86 (2H, s), 6.91 (1H, br s), 7.22 (2H, d, J=8 Hz), 7.40 (2H, br d, J=8 Hz), 7.79 (1H, s)

### Example 77

### [4-(3-ethyl-2,6-dioxo-3-piperidinyl)phenyl]carbamic acid(2-amino-2-methyl-1-oxopropoxy)methyl ester hydrochloride

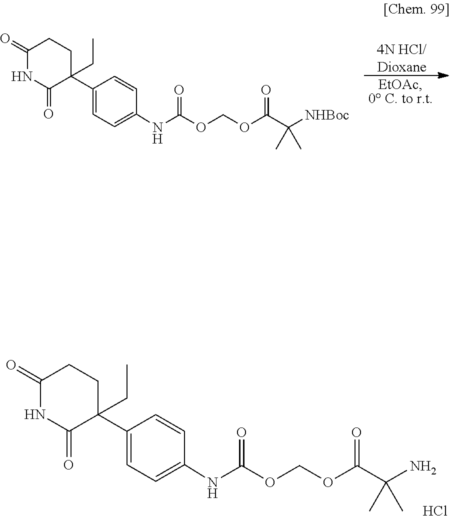 [SMILES: CCC1(c2ccc(NC(=O)OCOC(=O)C(C)(C)NC(=O)OC(C)(C)C)cc2)CCC(=O)NC1=O ; "EtOAc,0° C. to r.t." ; "4N HCl/Dioxane" ; CCC1(c2ccc(NC(=O)OCOC(=O)C(C)(C)N)cc2)CCC(=O)NC1=O ; Cl]

1.5 mL of 4 N hydrochloric acid/dioxane solution was added to an ethyl acetate solution of 90 mg (0.18 mmol) of [4-[3-ethyl-2,6-dioxo-3-piperidinyl)phenyl]carbamic acid[2-[[(1,1-dimethylethoxy)carbonyl]amino]-2-methyl-1-oxopropoxy]methyl ester, and the reaction solution was left to stand still for 10 hours. The solvent was distilled off under reduced pressure, diisopropyl ether was added to the residue and stirred. The precipitated crystals were filtered to obtain 64 mg (0.15 mmol) (83%). of the title compound.

1H-NMR (DMSO-d₆, δ): 0.75 (3H, t, J=7 Hz), 1.45 (3H, s), 1.49 (3H, s), 1.73-1.91 (2H, m), 2.06-2.20 (2H, m), 2.27-2.38 (1H, m), 2.41-2.48 (1H, m), 5.88 (2H, s), 7.24 (2H, d, J=8 Hz), 7.47 (2H, br d, J=8 Hz), 8.73 (3H, br s), 10.15 (1H, br s), 10.82 (1H, s)

### Example 78

### [4-[3-ethyl-2,6-dioxo-3-piperidinyl)phenyl]carbamic acid[2-[[[(1,1-dimethylethoxy)carbonyl]amino]methyl]-3-methyl-1-oxobutoxy]methyl ester

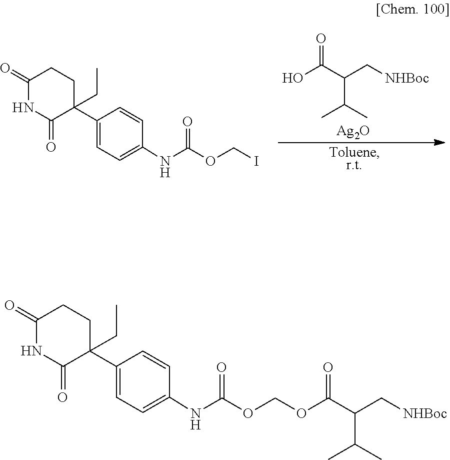

1127 mg (4.9 mmol) of silver oxide (I) was added to a solution of 1874 mg (8.1 mmol) of 2-[[[(1,1-dimethylethoxy)carbonyl]amino]methyl]-3-methyl butanoic acid in 20 mL of acetonitrile. Under an argon stream, 10 mL of water was added to the mixed liquid and stirred at 70° C. for 1 hour. The reaction solution was filtered through celite, and the filtrate was condensed under reduced pressure. The residue was dried with a vacuum pump to obtain 1875 mg of 2-[[[(1,1-dimethylethoxy)carbonyl]amino]methyl]-3-methyl butanoic acid silver salt. To a suspension of 360 mg (1.1 mmol) of the obtained 2-[[[(1,1-dimethylethoxy)carbonyl]amino]methyl]-3-methyl butanoic acid silver salt in dry toluene, a solution of 296 mg (0.71 mmol) of [4-(3-ethyl-2,6-dioxo-3-piperidinyl)phenyl]carbamic acid iodomethyl ester in dry toluene was added. The resultant solution was stirred at room temperature for 5 hours. Ethyl acetate was added to the reaction solution, the insoluble matter was removed by filtration, and the solvent was distilled off under reduced pressure. The residue was purified by silica gel column chromatography (15% to 80% ethyl acetate/toluene) to obtain 48 mg (0.09 mmol) (13%) of the title compound.

¹H-NMR (CDCl₃, δ): 0.87 (3H, t, J=7 Hz), 0.93-1.04 (6H, m), 1.35-1.46 (9H, m), 1.90 (1H, dq, J=14, 7 Hz), 1.95-2.08 (2H, m), 2.17-2.27 (1H, m), 2.32-2.45 (2H, m), 2.47-2.54 (1H, m), 2.56-2.66 (1H, m), 3.19-3.32 (1H, m), 3.40-3.54 (1H, m), 4.84 (1H, br s), 5.80-5.89 (2H, m), 6.91 (1H, br s), 7.24 (2H, d, J=9 Hz), 7.36-7.46 (2H, m), 7.81 (1H, s)

### Example 79

### [4-(3-ethyl-2,6-dioxo-3-piperidinyl)phenyl]carbamic acid[2-(aminomethyl)-3-methyl-1-oxobutoxy]methyl ester hydrochloride

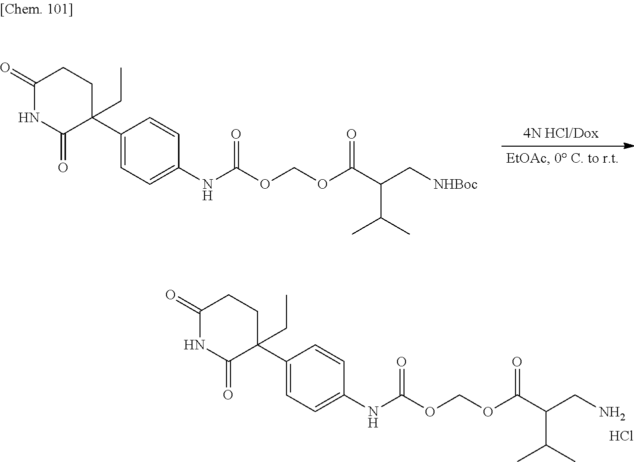 [SMILES: CCC1(c2ccc(NC(=O)OCOC(=O)C(CNC(=O)OC(C)(C)C)C(C)C)cc2)CCC(=O)NC1=O ; CCC1(c2ccc(NC(=O)OCOC(=O)C(CN)C(C)C)cc2)CCC(=O)NC1=O ; Cl]

0.6 mL of 4 N hydrochloric acid/dioxane solution was added to an ethyl acetate solution of 48 mg (0.09 mmol) of [4-[3-ethyl-2,6-dioxo-3-piperidinyl)phenyl]carbamic acid[2-[[[(1,1-dimethylethoxy)carbonyl]amino]methyl]-3-methyl-1-oxobutoxy]methyl ester, and the reaction solution was left to stand still for 10 hours. The solvent was distilled off under reduced pressure, diisopropyl ether was added to the residue and stirred. The precipitated crystals were filtered to obtain 34 mg (0.08 mmol) (83%). of the title compound.

1H-NMR (DMSO-d₆, δ): 0.75 (3H, t, J=7 Hz), 0.85-0.96 (6H, m), 1.71-1.91 (2H, m), 1.93-2.05 (1H, m), 2.08-2.18 (2H, m), 2.28-2.39 (1H, m), 2.40-2.48 (1H, m), 2.61-2.70 (1H, m), 2.91-2.99 (1H, m), 3.01-3.14 (1H, m), 5.75 (1H, d, J=6 Hz), 5.86 (1H, d, J=6 Hz), 7.23 (2H, d, J=9 Hz), 7.46 (2H, br d, J=8 Hz), 7.99 (3H, br s), 10.08 (1H, br s), 10.82 (1H, s)

### Example 80

### [2-[[[(aminoglutethimide-carbonyl)oxy]methoxy]carbonyl]-3-methylbutyl]amino-chondroitin sulfate conjugate

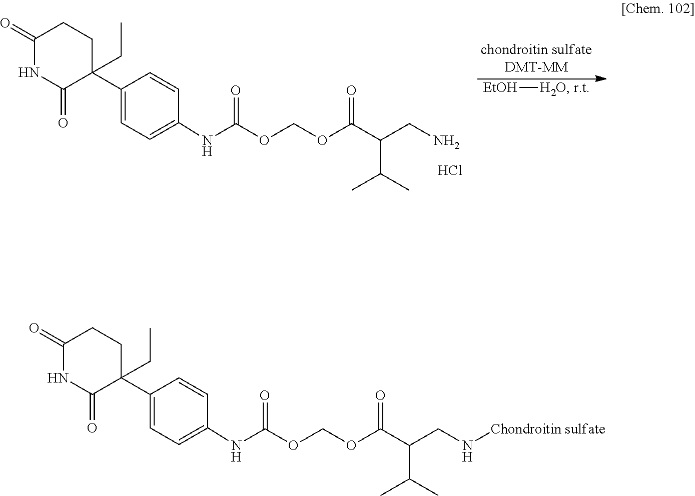

150 mg (0.3 mmol) of sodium chondroitin sulfate was dissolved in 3 mL of water, and 3 mL of ethanol was added dropwise thereto under stirring. To the mixed liquid, 50% ethanol aqueous solution of 27 mg (0.06 mmol) of [4-(3-ethyl-2,6-dioxo-3-piperidinyl)phenyl]carbamic acid[2-(aminomethyl)-3-methyl-1-oxobutoxy]methyl ester hydrochloride was added. Then, 50% ethanol aqueous solution of 27 mg (0.06 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 1 mL) until immediately before the reaction solution became cloudy. The reaction solution was added dropwise to 9 mL of 90% ethanol under stirring and left to stand still. The supernatant was decanted, and then 10 mL of 90% ethanol was added thereto and stirred. Precipitates were isolated using a centrifuge, washing with 90% ethanol was performed two times, washing with ethanol was performed two times, and washing with diethyl ether was further performed two times. The obtained precipitates were dried overnight using a vacuum pump to obtain 151 mg of the title compound. Based on values of integral in ¹H-NMR, the introduction ratio of aminoglutethimide per unit of whole disaccharide (glucuronic acid) of chondroitin sulfate was 15%.

### Example 81

### [3-[[(etilefrine-carbonyl)oxy]methoxy]-3-oxopropyl]amino-chondroitin sulfate (20,000) conjugate

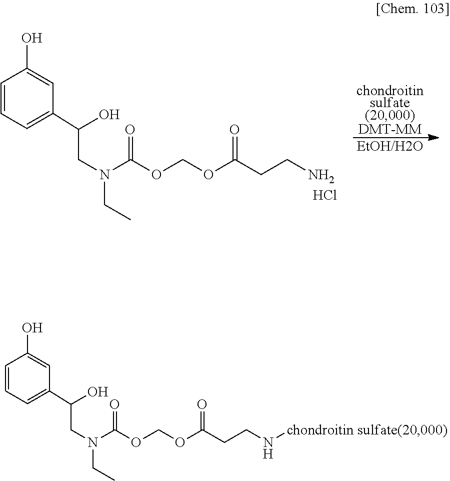

200 mg (0.4 mmol) of sodium chondroitin sulfate (20,000) was dissolved in 4 mL of water, and 4 mL of ethanol was added dropwise thereto under stirring. To the mixed liquid, 50% ethanol aqueous solution of 71 mg (0.2 mmol) of N-ethyl-N-[2-hydroxy-2-(3-hydroxyphenyl)ethyl]carbamic acid(3-amino-1-oxopropoxy)methyl ester hydrochloride was added. Then, 50% ethanol aqueous solution of 90 mg (0.2 mmol) of 4-(4,6-dimethoxy-1,3,5-triazin-2-yl)-4-methylmorpholinium chloride (DMT-MM) was rapidly added thereto and stirred at room temperature overnight. 100 μL of 20% sodium chloride aqueous solution was added to the reaction solution, and ethanol was further added dropwise (about 5 mL) until immediately before the reaction solution became cloudy. The reaction solution was added dropwise to 8 mL of 90% ethanol under stirring. The supernatant was decanted, and the precipitates were dissolved in 16 mL of a 50% ethanol aqueous solution. 50 mL of ethanol was added to the resultant solution under stirring, and then the supernatant was decanted. 10 mL of 90% ethanol was added thereto, precipitates were isolated using a centrifuge, washing with 90% ethanol was performed two times, washing with ethanol was performed two times, and washing with diethyl ether was further performed two times. The obtained precipitates were dried overnight with a vacuum pump to obtain 233 mg of the title compound. Based on values of integral in 1H-NMR, the introduction ratio of etilefrine per unit of whole disaccharide (glucuronic acid) of chondroitin sulfate was 37.4%.

### Example 82

### [3-[[(etilefrine-carbonyl)oxy]methoxy]-3-oxopropyl]amino-chondroitin sulfate (40,000) conjugate

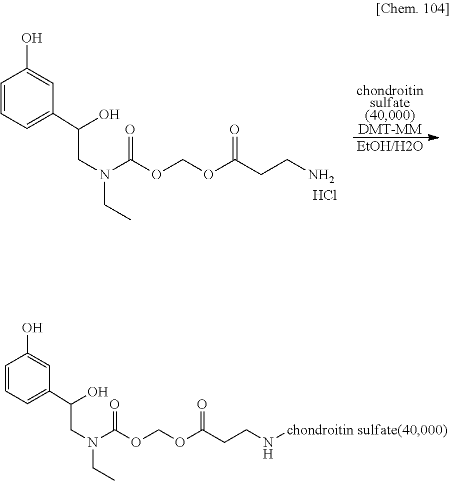

200 mg (0.39 mmol) of sodium chondroitin sulfate (40,000) was dissolved in 4 mL of water, and 4 mL of ethanol was added dropwise to the resultant solution under stirring. To the mixed liquid, 50% ethanol aqueous solution of 72 mg (0.20 mmol) of N-ethyl-N-[2-hydroxy-2-(3-hydroxyphenyl)ethyl]carbamic acid(3-amino-1-oxopropoxy)methyl ester hydrochloride was added. Then, 50% ethanol aqueous solution of 91 mg (0.20 mmol) of 4-(4,6-dimethoxy-1,3,5-triazin-2-yl)-4-methylmorpholinium chloride (DMT-MM) was rapidly added thereto and stirred at room temperature overnight. 333 μL of 20% sodium chloride aqueous solution was added to the reaction solution, and ethanol was further added dropwise (3 mL) until immediately before the reaction solution became cloudy. The reaction solution was added dropwise to 13 mL of ethanol under stirring. Precipitates were isolated using a centrifuge, washing with 90% ethanol was performed two times, washing with ethanol was performed two times, and washing with diethyl ether was further performed one time. The obtained precipitates were dried overnight with a vacuum pump to obtain 239 mg of the title compound. Based on values of integral in 1H-NMR, the introduction ratio of etilefrine per unit of whole disaccharide (glucuronic acid) of chondroitin sulfate was 34.7%.

### Example 83

### [3-[[(etilefrine-carbonyl)oxy]methoxy]-3-oxopropyl]amino-hyaluronic acid (650,000) conjugate

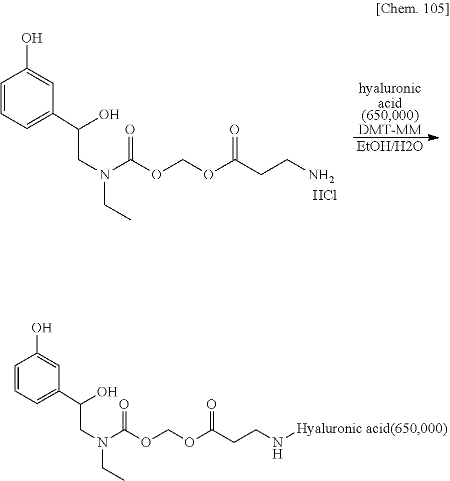

10 mL of ethanol was added dropwise to 10 g (0.25 mmol) of 1% sodium hyaluronate (650,000) aqueous solution under stirring. To the mixed liquid, 50% ethanol aqueous solution of 18 mg (0.05 mmol) of N-ethyl-N-[2-hydroxy-2-(3-hydroxyphenyl)ethyl]carbamic acid(3-amino-1-oxopropoxy)methyl ester hydrochloride was added. Then, 50% ethanol aqueous solution of 11 mg (0.02 mmol) of 4-(4,6-dimethoxy-1,3,5-triazin-2-yl)-4-methylmorpholinium chloride (DMT-MM) was rapidly added thereto and stirred at room temperature overnight. The reaction solution was dissolved by adding 500 mg of sodium chloride, and 30 mL of ethanol was added dropwise thereto to form precipitates. The supernatant was decanted, and then 10 mL of ethanol was added to the precipitates and stirred. The supernatant was decanted again. Then, the precipitates were isolated using a centrifuge, washing with 90% ethanol was performed two times, washing with ethanol was performed two times, and washing with diethyl ether was performed two times. The obtained precipitates were dried overnight with a vacuum pump to obtain 83 mg of the title compound. Based on values of integral in 1H-NMR, the introduction ratio of etilefrine per unit of whole disaccharide (glucuronic acid) of hyaluronic acid was 9.1%.

### Example 84

### [3-[[(troxipide-carbonyl)oxy]methoxy]-3-oxopropyl]amino-chondroitin sulfate (20,000) conjugate

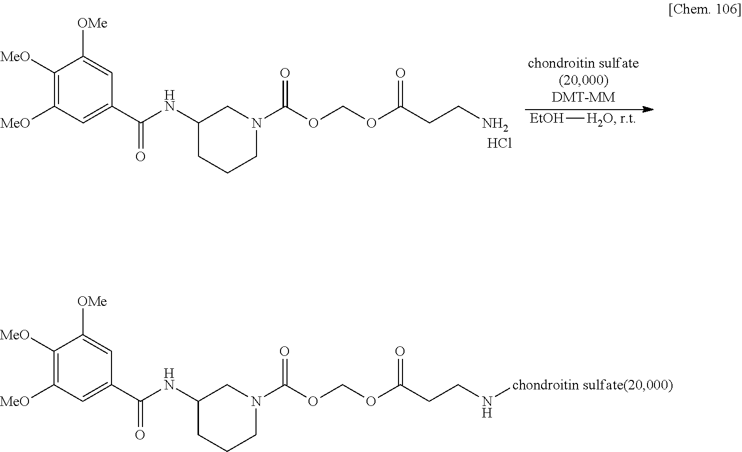

250 mg (0.49 mmol) of sodium chondroitin sulfate (20,000) was dissolved in 5 mL of water, and 5 mL of ethanol was added dropwise thereto under stirring. To the mixed liquid, 50% ethanol aqueous solution of 116 mg (0.24 mmol) of 3-[(3,4,5-trimethoxybenzoyl)amino]-1-piperidinecarboxylic acid[(3-amino-1-oxopropoxy)]methyl ester hydrochloride was added. Then, 50% ethanol aqueous solution of 113 mg (0.24 mmol) of 4-(4,6-dimethoxy-1,3,5-triazin-2-yl)-4-methylmorpholinium chloride (DMT-MM) was rapidly added thereto and stirred at room temperature overnight. 417 μL of 20% sodium chloride aqueous solution was added to the reaction solution, and ethanol was further added dropwise (4 mL) until immediately before the reaction solution became cloudy. The reaction solution was added dropwise to 15 mL of ethanol under stirring. Precipitates were isolated using a centrifuge, washing with 90% ethanol was performed two times, washing with ethanol was performed two times, and washing with diethyl ether was further performed one time. The obtained precipitates were dried overnight with a vacuum pump to obtain 321 mg of the title compound. Based on values of integral in 1H-NMR, the introduction ratio of troxipide per unit of whole disaccharide (glucuronic acid) of chondroitin sulfate was 35.7%.

### Example 85

### [3-[[(troxipide-carbonyl)oxy]methoxy]-3-oxopropyl]amino-chondroitin sulfate (40,000) conjugate

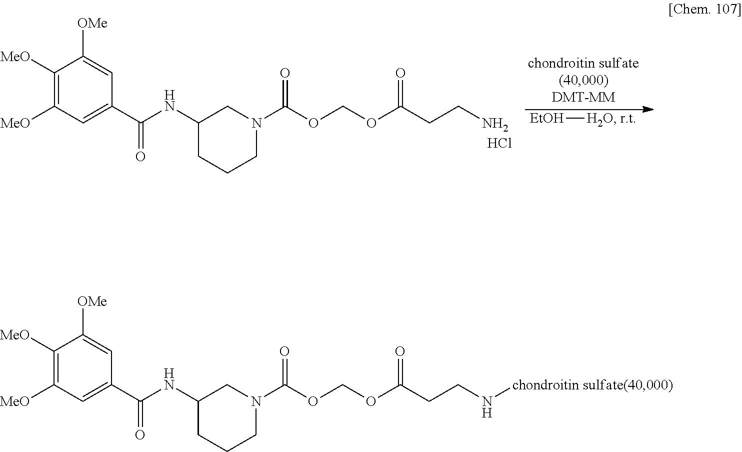

200 mg (0.39 mmol) of sodium chondroitin sulfate (40,000) was dissolved in 4 mL of water, and 4 mL of ethanol was added dropwise to the resultant solution under stirring. To the mixed liquid, 50% ethanol aqueous solution of 94 mg (0.20 mmol) of 3-[(3,4,5-trimethoxybenzoyl)amino]-1-piperidinecarboxylic acid[(3-amino-1-oxopropoxy)]methyl ester hydrochloride was added. Then, 50% ethanol aqueous solution of 91 mg (0.20 mmol) of 4-(4,6-dimethoxy-1,3,5-triazin-2-yl)-4-methylmorpholinium chloride (DMT-MM) was rapidly added thereto and stirred at room temperature overnight. 333 μL of 20% sodium chloride aqueous solution was added to the reaction solution, and ethanol was further added dropwise (3 mL) until immediately before the reaction solution became cloudy. The reaction solution was added dropwise to 13 mL of ethanol under stirring. Precipitates were isolated using a centrifuge, washing with 90% ethanol was performed two times, washing with ethanol was performed two times, and washing with diethyl ether was further performed one time. The obtained precipitates were dried overnight with a vacuum pump to obtain 240 mg of the title compound. Based on values of integral in 1H-NMR, the introduction ratio of troxipide per unit of whole disaccharide (glucuronic acid) of chondroitin sulfate was 39.6%.

### Example 86

### [3-[[(troxipide-carbonyl)oxy]methoxy]-3-oxopropyl]amino-hyaluronic acid (70,000) conjugate

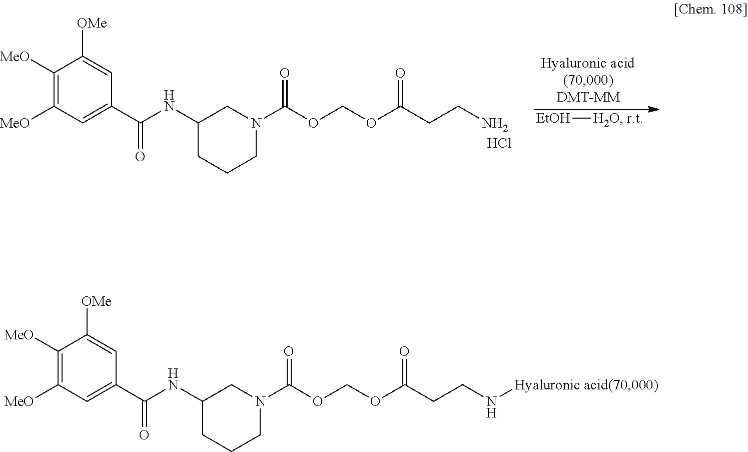

15 mL of ethanol was added dropwise to 15 g (0.37 mmol) of 1% sodium hyaluronate (70,000) aqueous solution under stirring. To the mixed liquid, 50% ethanol aqueous solution of 53 me (0.11 mmol) of 3-1 (3.4.5-trimethoxybenzoyl)amino]-1-piperidinecarboxylic acid[(3-amino-1-oxopropoxy)]methyl ester hydrochloride was added. Then, 50% ethanol aqueous solution of 25 mg (0.06 mmol) of 4-(4,6-dimethoxy-1,3,5-triazin-2-yl)-4-methylmorpholinium chloride (DMT-MM) was rapidly added thereto and stirred at room temperature overnight. The reaction solution was dissolved by adding 750 mg of sodium chloride, and 45 mL of ethanol was added dropwise thereto to form precipitates. The supernatant was decanted, and then 15 mL of ethanol was added to the precipitates and stirred. The supernatant was decanted again. Then, the precipitates were isolated using a centrifuge, washing with 90% ethanol was performed two times, washing with ethanol was performed two times, and washing with diethyl ether was performed two times. The obtained precipitates were dried overnight with a vacuum pump to obtain 158 mg of the title compound. Based on values of integral in 1H-NMR, the introduction ratio of troxipide per unit of whole disaccharide (glucuronic acid) of hyaluronic acid was 12.8%.

### Example 87

### [3-[[(troxipide-carbonyl)oxy]methoxy]-3-oxopropyl]amino-hyaluronic acid (100,000) conjugate

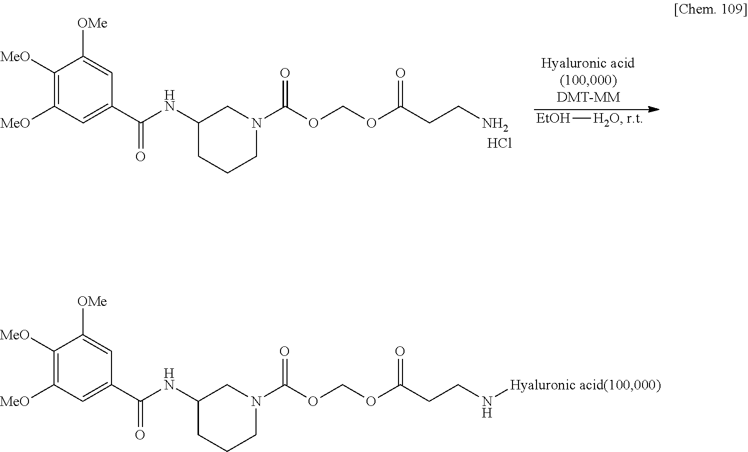

15 mL of ethanol was added dropwise to 15 g (0.37 mmol) of 1% sodium hyaluronate (100,000) aqueous solution under stirring. To the mixed liquid, 50% ethanol aqueous solution of 53 mg (0.11 mmol) of 3-[(3,4,5-trimethoxybenzoyl)amino]-1-piperidinecarboxylic acid[(3-amino-1-oxopropoxy)]methyl ester hydrochloride was added. Then, 50% ethanol aqueous solution of 25 mg (0.06 mmol) of 4-(4,6-dimethoxy-1,3,5-triazin-2-yl)-4-methylmorpholinium chloride (DMT-MM) was rapidly added thereto and stirred at room temperature overnight. The reaction solution was dissolved by adding 750 mg of sodium chloride, and 45 mL of ethanol was added dropwise thereto to form precipitates. The supernatant was decanted, and then 15 mL of ethanol was added to the precipitates and stirred. The supernatant was decanted again. Then, the precipitates were isolated using a centrifuge, washing with 90% ethanol was performed two times, washing with ethanol was performed two times, and washing with diethyl ether was performed two times. The obtained precipitates were dried overnight using a vacuum pump to obtain 139 mg of the title compound. Based on values of integral in 1H-NMR, the introduction ratio of troxipide per unit of whole disaccharide (glucuronic acid) of hyaluronic acid was 13.5%.

### Example 88

### [3-[[(troxipide-carbonyl)oxy]methoxy]-3-oxopropyl]amino-carboxymethyl cellulose conjugate

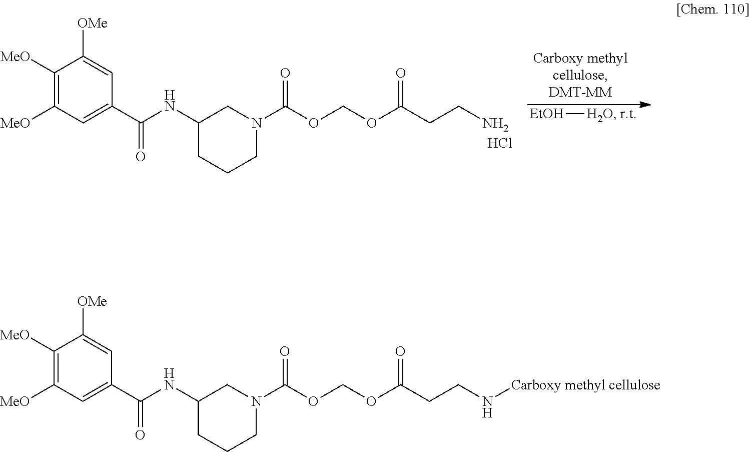

20 mL of ethanol was added dropwise to 20 g (0.88 mmol) of 1% carboxymethyl cellulose aqueous solution under stirring. To the mixed liquid, 50% ethanol aqueous solution of 21 mg (0.04 mmol) of 3-[(3,4,5-trimethoxybenzoyl)amino]-1-piperidinecarboxylic acid[(3-amino-1-oxopropoxy)]methyl ester hydrochloride salt was added. Then, 50% ethanol aqueous solution of 25 mg (0.04 mmol) of 4-(4,6-dimethoxy-1,3,5-triazin-2-yl)-4-methylmorpholinium chloride (DMT-MM) was rapidly added thereto and stirred at room temperature overnight. The reaction solution was dissolved by adding 2 mL of 20% sodium chloride aqueous solution, and 60 mL of ethanol was added dropwise thereto to form precipitates. The supernatant was decanted, and then 30 mL of 90% ethanol was added to the precipitates and stirred. The supernatant was decanted again. Then, the precipitates were isolated using a centrifuge, washing with 90% ethanol was performed two times, washing with ethanol was performed two times, and washing with diethyl ether was performed two times. The obtained precipitates were dried overnight using a vacuum pump to obtain 201 mg of the title compound. According to the measurement result (255 nm) of the spectrophotometer, the introduction ratio of troxipide was 2%.

### Example 89

### [3-[[(troxipide-carbonyl)oxy]methoxy]-3-oxopropyl]amino-alginic acid conjugate

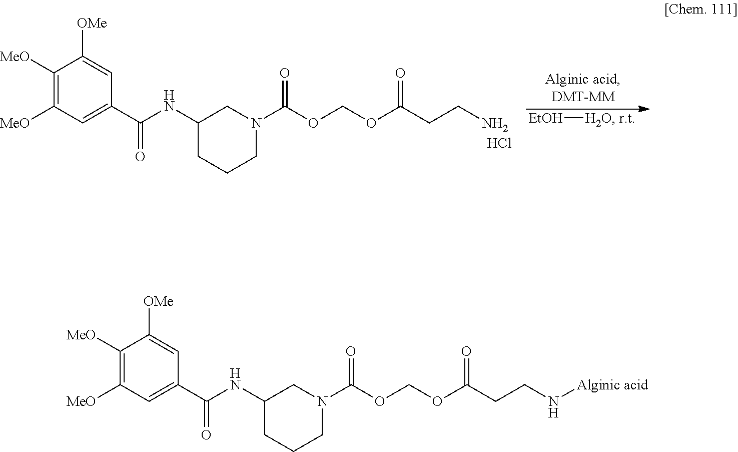

20 mL of ethanol was added dropwise to 20 g (1.01 mmol) of 1% sodium alginate aqueous solution under stirring. To the mixed liquid, 50% ethanol aqueous solution of 24 mg (0.05 mmol) of 3-[(3,4,5-trimethoxybenzoyl)amino]-1-piperidinecarboxylic acid[(3-amino-1-oxopropoxy)]methyl ester hydrochloride was added. Then, 50% ethanol aqueous solution of 23 mg (0.05 mmol) of 4-(4,6-dimethoxy-1,3,5-triazin-2-yl)-4-methylmorpholinium chloride (DMT-MM) was rapidly added thereto and stirred at room temperature overnight. The reaction solution was dissolved by adding 3 mL of a 20% sodium chloride aqueous solution, and 400 mL of acetone was added dropwise thereto to form precipitates. The supernatant was decanted, and then 100 mL of 90% acetone was added to the precipitates and stirred. The supernatant was decanted again. Then, the precipitates were isolated using a centrifuge, and washing with 90% acetone was performed two times, washing with acetone was performed two times, and washing with diethyl ether was performed two times. The obtained precipitates were dried overnight with a vacuum pump to obtain 183 mg of the title compound. According to the measurement result (255 nm) of the spectrophotometer, the introduction ratio of troxipide was 2%.

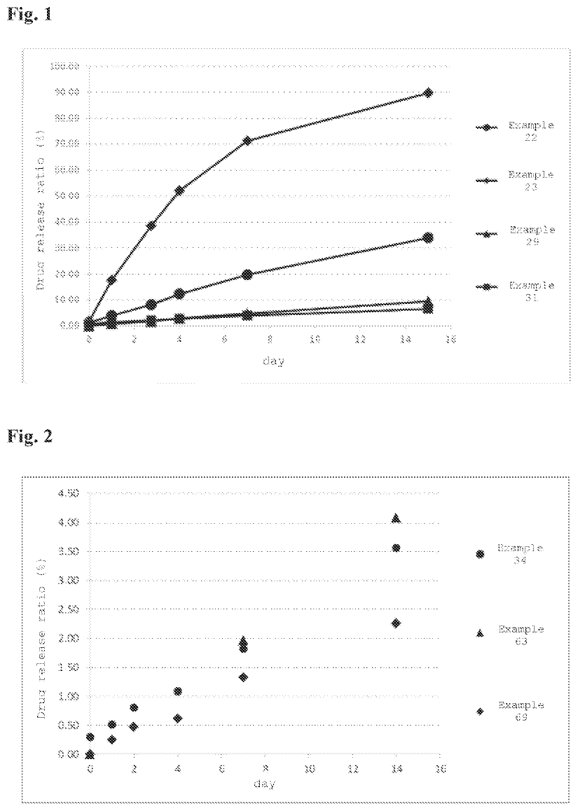

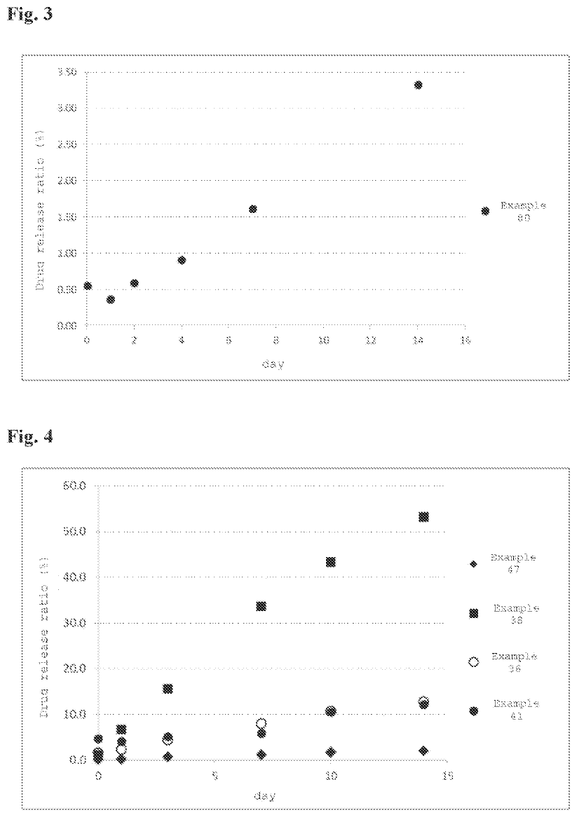

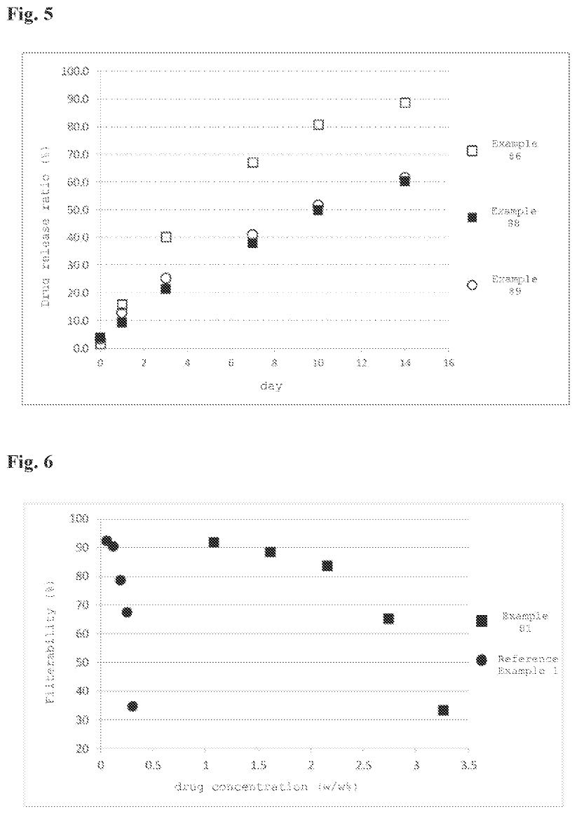

### Test Example 1 Drug Release Test of Drug-Polymer Conjugate

[Operation] Each evaluation polymer conjugate presented in Table 1 was dissolved in a concentration of 1.5 mg/mL in a sodium phosphate buffer solution having a pH of 7.0 and then dispensed. The state immediately after dissolving was defined as an initial state (on day 0 of storage), the reaction solution was diluted to 0.5 mg/mL with an aqueous solution of acetonitrile/physiological saline (1:1), the drug-polymer conjugate and the release drug present in the solution were analyzed by SEC-HPLC. Other dispensed injection liquid was stored under the condition of 36° C. immediately after dissolving, and the dispensed injection liquid after each time elapsed was analyzed in the similar manner. From a ratio of the release drug peak area and the drug-polymer conjugate peak area at each time point which had been obtained in this way, a drug release ratio (%) was calculated. The relation between the time and the drug release ratio (Drug Release) is as shown in FIGS. 1, 2, 3, 4, and 5.

The HPLC conditions are as follows.

Column: TSGgel α-3000 (7.8 mm×300 mm)

Flow rate: 0.5 mL/min

Temperature: 35° C.

Mobile phase: acetonitrile/physiological saline=1/2

Drug release ratio=release drug peak area/(release drug peak area+conjugate peak area)×100

[Results] As shown in FIGS. 1, 2, 3, 4, and 5, the drug release amount of each novel conjugate of a primary amine compound or a secondary amine compound with an acidic polysaccharide polymer (i.e., the compound of the present invention) is increased depending on the time. As a result, it is possible to impart a sustained-release property to the conjugate regardless of the structure of the primary amine compound or the secondary amine compound having bioactivity.

As shown in FIGS. 1, 2, 3, 4, and 5, the release rate of the primary amine compound or the secondary amine compound having bioactivity is very finely and widely controlled by the linker structure.

TABLE 1

| Number of | | |
|---|---|---|
| example | Compound name | Structural formula |
| Example 22 | [3-[[(troxipide- carbonyl)oxy]methoxy]-3- oxopropyl]amino- chondroitin sulfate conjugate | 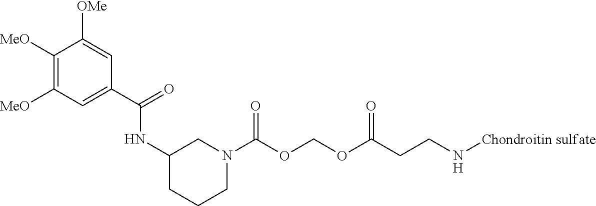 |
| Example 23 | [2-[[(troxipide- carbonyl)oxy]methoxy]-2- oxoethyl]amino- chondroitin sulfate conjugate | 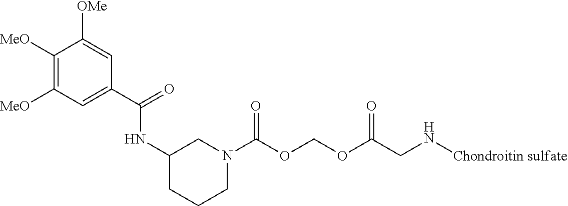 |
| Example 29 | [5-[[(methoxyphenamine- carbonyl)oxy]methoxy]-5- oxopentanoyl]amino- chondroitin sulfate conjugate | 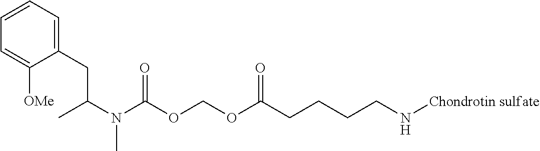 |
| Example 31 | [4-[1- [(methoxyphenamine- carbonyl)oxy]ethoxy]-4- oxobutanoyl]amino- chondroitin sulfate conjugate | 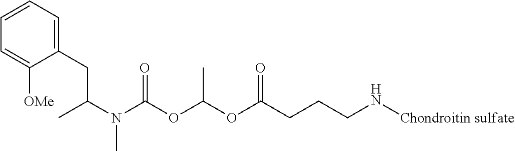 |

TABLE 2

| Number of | | |
|---|---|---|
| example | Compound name | Structural formula |
| Example 34 | [3-[1-(etilefrine- carbonyl)oxy]- 2-methylpropoxy]-3-oxo-2- methylpropyl]amino- chondroitin sulfate conjugate | 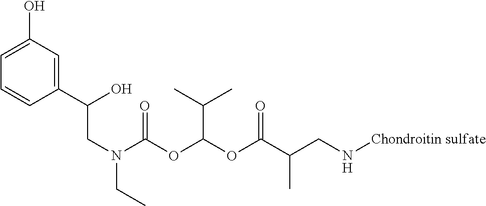 |
| Example 63 | [6-[1-[(mexiletine- carbonyl)oxy]ethoxy]-6- oxohexyl]amino- chondroitin sulfate conjugate | 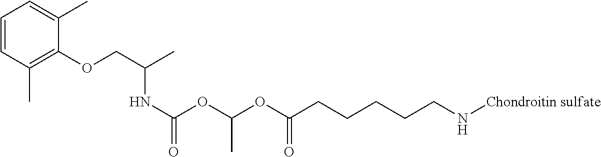 |
| Example 69 | [6-[1-[(fluvoxamine- carbonyl)oxy]ethoxy]-6- oxohexyl]amino- chondroitin sulfate conjugate | 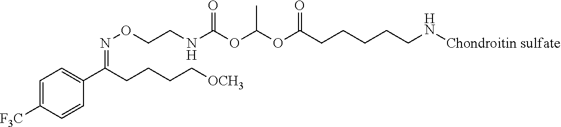 |

TABLE 3

| Number of | | |
|---|---|---|
| example | Compound name | Structural formula |
| Example 80 | [3-methyl-2- [[[(aminoglutethimide- carbonyl)oxy]methoxy] carbonyl]butyl] amino-chondroitin sulfate conjugate | 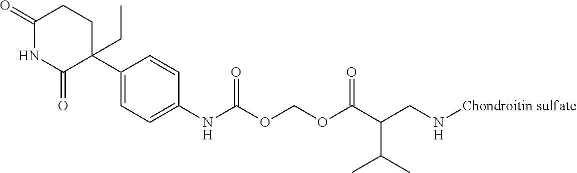 |

TABLE 4

| Number of | | |
|---|---|---|
| example | Compound name | Structural formula |
| Example 47 | [1-[[1-[(methoxyphenamine- carbonyl)oxy]ethoxy] carbonyl]cyclopropyl] amino-chondroitin sulfate conjugate | 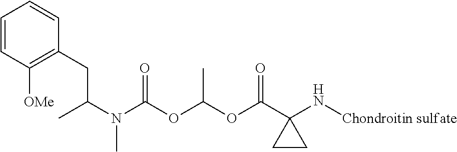 |
| Example 38 | [2-[1-[(troxipide- carbonyl)oxy]ethoxy]-2- oxoethyl]amino- chondroitin sulfate conjugate | 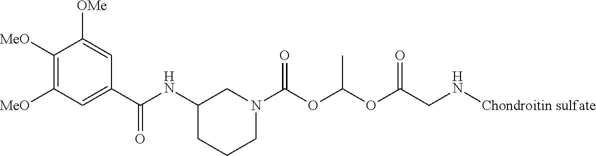 |
| Example 36 | [3-[1-[(troxipide- carbonyl)oxy]ethoxy]-3- oxopropyl]amino- chondroitin sulfate conjugate | 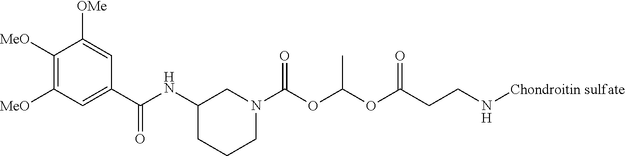 |
| Example 41 | [1,1-dimethyl-2- oxo-2-[1-[(troxipide- carbonyl)oxy] ethoxy]ethyl]amino- chondroitin sulfate conjugate | 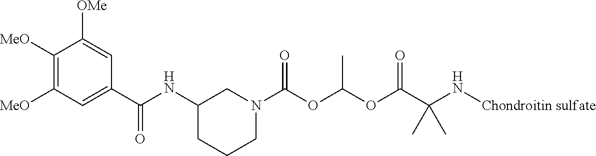 |

TABLE 5

| Number of | | |
|---|---|---|
| example | Compound name | Structural formula |
| Example 86 | [3-[[(troxipide- carbonyl)oxy]methoxy]-3- oxopropyl]amino-hyaluronic acid (70,000) conjugate | 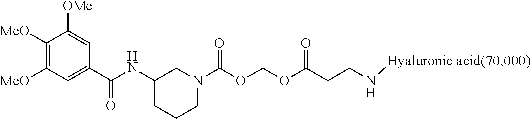 |
| Example 88 | [3-[[(troxipide- carbonyl)oxy]methoxy]-3- oxopropyl]amino- carboxymethyl cellulose conjugate | 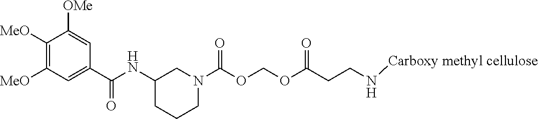 |
| Example 89 | [3-[[(troxipide- carbonyl)oxy]methoxy]-3- oxopropyl]amino-alginic acid conjugate | 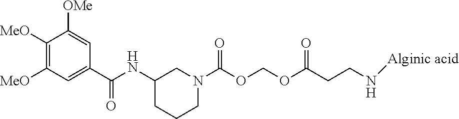 |

### Test Example 2 Comparative Filterability Test of Drug-Polymer Conjugate

### [Operation]

Chondroitin sulfate (CS)-conjugates and polyethylene glycol (PEG)-conjugates shown in Tables 6, 8, and 10 were weighed in an Eppendorf tube, and distilled water was added to the respective samples so as to give the conjugate concentrations shown in Tables 7, 9, and 11. The reaction solutions were stirred with a vortex mixer for 30 seconds and then centrifuged at 2000 G for 60 seconds. After repeating this process two times, the reaction solutions were left to stand still at room temperature for 1 hour. The reaction solutions were again stirred with the vortex mixer for 30 seconds and then centrifuged at 2000G for 60 seconds to prepare lysates.

About 70 to 90 mg of each of the dissolved samples was weighed in a polyvinylidene fluoride (PVDF) centrifugal filter unit having a pore size of 0.22 μm and centrifuged at 25° C. and 12000G for 90 minutes. The liquid passing through the filter was weighed, and the filter passing rate (wt %) was calculated.

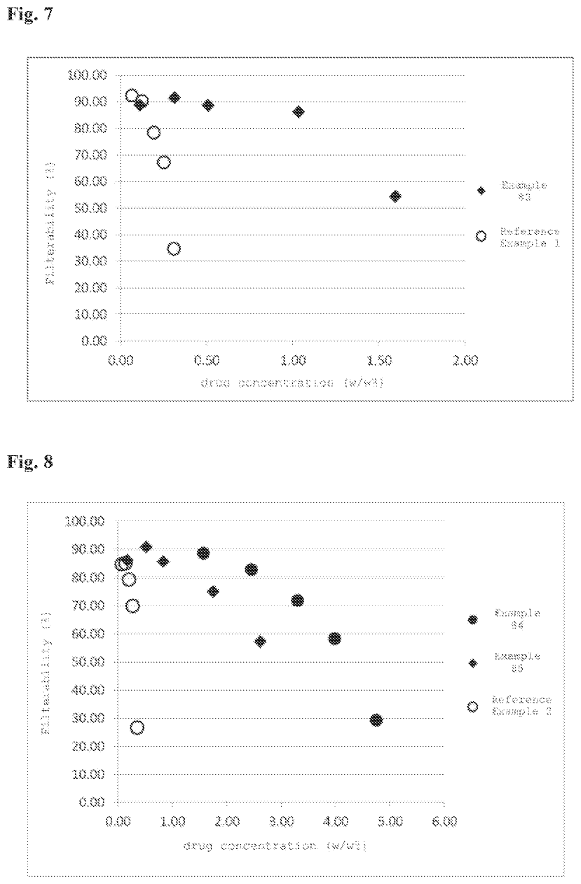

The relation between the drug concentration in the solution calculated from the introduction ratio of each of the samples and the filter passing rate is as shown in FIGS. 6, 7, and 8.

[Results]

In the case of a solution having filterability, dust removal, bacteria elimination, sterilization, and the like can be performed by filter filtration. In particular, the filterability of a filter having a pore size of 0.22 μm used for sterilization by filtration is one of the important parameters in producing a solution formulation such as an eye drop or an injection.

As shown in FIGS. 6, 7, and 8, in the case of the conjugate of a primary amine or secondary amine compound with an acidic polysaccharide (i.e., the compound of the present invention), it is possible to prepare a solution that can be filtered through a 0.22 μm filter at a higher drug concentration than the PEG conjugate. Further, even if the acidic polysaccharide conjugate has a high polymer molecular weight, it is possible to prepare a solution having filterability at a higher drug concentration than the PEG conjugate, and to provide a solution formulation having a high molecular weight and a high drug concentration.

TABLE 6

| Number of | | Molecular | |
|---|---|---|---|
| example | Compound name | weight | Structural formula |
| Example 81 | [3-[[(etilefrine- carbonyl)oxy] methoxy]-3- oxopropyl]amino- chondroitin sulfate (20,000) conjugate | 20 KDa | 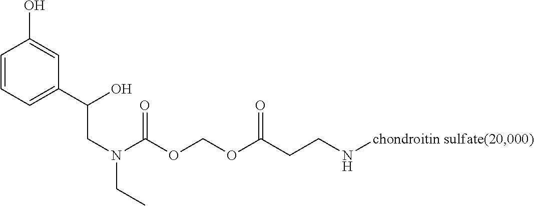 |
| Reference Example 1 | [3-[[(etilefrine- carbonyl)oxy] methoxy]-3- oxopropyl]amino- methoxypolyethylene glycol acetic acid conjugate | 20 KDa | 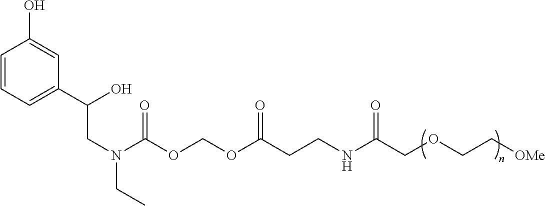 |

TABLE 7

| | | | Sample | |
|---|---|---|---|---|
| | | | weight | |
| | | | (mg)/ | |
| | | Concentration | Solution | |
| Sample | | wt % | weight | Concentration |
| No. | Sample | (conjugate) | 100 mg | wt % |
| E01 | CS | 10 | 10.0 | 1.10 |
| E02 | (20K)-Etilefrine | 15 | 15.0 | 1.64 |
| E03 | Example 81 | 20 | 20.0 | 2.19 |
| E04 | Introduction | 25 | 25.0 | 2.74 |
| E05 | ratio 37.4% | 30 | 30.0 | 3.29 |
| E06 | mPEG (20K)- | 10 | 10.0 | 0.06 |
| E07 | Etilefrine | 20 | 20.0 | 0.12 |
| E08 | Reference | 30 | 30.0 | 0.18 |
| E09 | Example 1 | 40 | 40.0 | 0.24 |
| E10 | Introduction | 50 | 50.0 | 0.31 |
| E11 | ratio 68.6% | 60 | 60.0 | 0.37 |

TABLE 8

| Number of | | Molecular | |
|---|---|---|---|
| example | Compound name | weight | Structural formula |
| Example 82 | [3-[[(etilefrine- carbonyl)oxy] methoxy]-3- oxopropyl]amino- chondroitin sulfate (40,000) conjugate | 40 KDa | 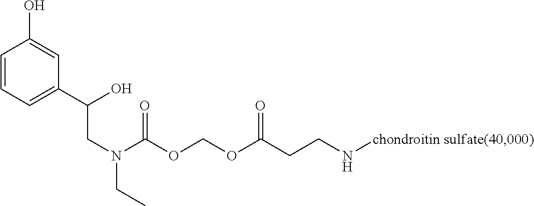 |

TABLE 9

| | | Concentration | Concentration |
|---|---|---|---|
| Sample | | wt % | wt % |
| No. | Sample | (conjugate) | (Etilefrine) |
| E01 | CS (40K)- | 1.1 | 0.11 |
| E02 | Etilefrine | 3.0 | 0.31 |
| E03 | Example 82 | 4.9 | 0.51 |
| E04 | Introduction | 10.0 | 1.03 |
| E05 | ratio 34.7% | 15.4 | 1.59 |

TABLE 10

| Number of | | Molecular | |
|---|---|---|---|
| example | Compound name | weight | Structural formula |
| Example 84 | [3-[[(troxipide- carbonyl)oxy] methoxy]-3- oxopropyl]amino- chondroitin sulfate (20,000) conjugate | 20 KDa | 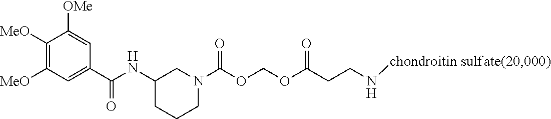 |
| Example 85 | [3-[[(troxipide- carbonyl)oxy] methoxy]-3- oxopropyl]amino- chondroitin sulfate (40,000) conjugate | 40 KDa | 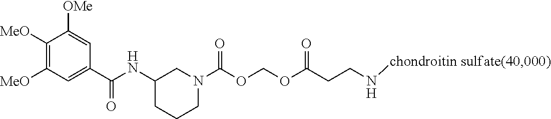 |
| Reference Example 2 | 2-[[3-[[(troxipide- carbonyl)oxy] methoxy]-3- oxopropyl]amino]- 2-oxoethyl- methoxypolyethylene glycol (20,000) conjugate | 20 KDa | 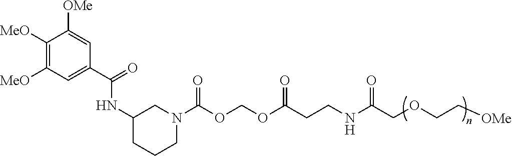 |

TABLE 11

| | | Concentration | Concentration |
|---|---|---|---|
| Sample | | wt % | wt % |
| No. | Sample | (conjugate) | (Troxipide) |
| E01 | CS (20K)- | 9.9 | 1.58 |
| E02 | Troxipide | 15.3 | 2.46 |
| E03 | Example 84 | 20.6 | 3.31 |
| E04 | Introduction | 24.8 | 3.99 |
| E05 | ratio 35.7% | 29.7 | 4.77 |
| E06 | CS (40K)- | 1.0 | 0.18 |
| E07 | Troxipide | 3.0 | 0.53 |
| E08 | Example 85 | 4.8 | 0.85 |
| E09 | Introduction | 10.0 | 1.76 |
| E10 | ratio 39.6% | 15.0 | 2.63 |
| E11 | mPEG (20K)- | 10.0 | 0.07 |
| E12 | Troxipide | 19.7 | 0.14 |
| E13 | Reference | 29.2 | 0.21 |
| E14 | Example 2 | 39.3 | 0.28 |
| E15 | Introduction | 50.1 | 0.36 |
| | ratio 49.4% | | |

1. A compound represented by Formula (I) or a pharmaceutically acceptable salt thereof; 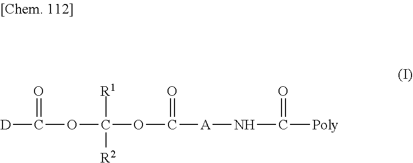
in Formula (I), D represents a residue of a primary or secondary amine compound DH excluding a hydrogen atom of a primary or secondary amino group; R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a substituted or unsubstituted bivalent hydrocarbon group and may contain one or more hetero atoms at a position except for both ends which are bonded to —C(═O)— or —NH—, the hetero atoms are each independently selected from the group consisting of —O—, —NH— which may have a substituent, and —S—; any two or three groups of R¹, R², and A may combine together to form a ring; Poly represents an acidic polysaccharide residue, and —C(═O)— adjacent to Poly is derived from a carboxy group of the acidic polysaccharide.

2. The compound according to claim 1 or a pharmaceutically acceptable salt thereof, wherein the Formula (I) is represented by the following Formula (II) 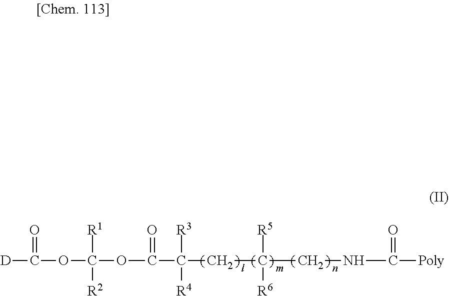
in Formula (II), D, R¹, R² and Poly are as defined in claim 1; R³, R⁴, R⁵, and R⁶ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three groups of R¹, R², R³, R⁴, R⁵, and R⁶ may combine together to form a ring; 1 and n are each independently 0, 1, or 2, and m is 0 or 1.

3. The compound according to claim 2 or a pharmaceutically acceptable salt thereof, wherein in Formula (I) or (II); R¹, R², R³, R⁴, R⁵, and R⁶ are each independently a hydrogen atom; a substituted or unsubstituted linear or branched alkyl group having carbon number of 1 to 6; a substituted or unsubstituted cycloalkyl group having carbon number of 3 to 8; a substituted or unsubstituted linear or branched alkenyl group having carbon number of 2 to 6; a substituted or unsubstituted cycloalkenyl group having carbon number of 3 to 8; a substituted or unsubstituted linear or branched alkynyl group having carbon number of 2 to 6; a substituted or unsubstituted monocyclic or polycyclic aromatic group having carbon number of 6 to 14; or a substituted or unsubstituted 3- to 8-membered heterocyclic group containing at least one of a nitrogen atom, an oxygen atom, or a sulfur atom as a ring-constituting atom.

4. The compound according to claim 2 or a pharmaceutically acceptable salt thereof, wherein in Formula (I) or (II), a substituent of alkyl, a substituent of cycloalkyl group, a substituent of alkenyl group, a substituent of cycloalkenyl group, a substituent of alkynyl group, a substituent of aromatic group, and a substituent of heterocyclic group in the groups represented by R¹, R², R³, R⁴, R⁵, and R⁶ are groups selected from a hydroxyl group, an alkyl group, a cycloalkyl group, an alkenyl group, a cycloalkenyl group, an alkynyl group, a halogen atom, an aromatic group, a heterocyclic group, an alkoxy group, a guanidino group, an alkylthio group, an alkoxycarbonyl group, an aryloxy group, an arylthio group, an acyl group, a substituted sulfonyl group, a heterocyclyloxy group, a heterocyclylthio group, an amide group, a ureido group, a carboxy group, a carbamoyl group, an oxo group, a thioxo group, a sulfamoyl group, a sulfo group, a cyano group, a nitro group, an acyloxy group, an azido group, a sulfonamide group, a mercapto group, an alkoxycarbonyl amino group, an aminocarbonyloxy group, a substituted sulfinyl group, a sulfamide group, an aminosulfonyloxy group, an alkoxysulfonyl amino group, a substituted sulfonyloxy group, an alkoxycarbonyl group, an alkoxycarbonyloxy group, an alkoxysulfonyl group, an Rx(Ry)N group, and an Rx(Ry)(Rz)N⁺ group, and Rx, Ry, and Rz are each independently selected from the group consisting of a hydrogen atom, an alkyl group, a cycloalkyl group, an alkenyl group, a cycloalkenyl group, an alkynyl group, an aromatic hydrocarbon group, or a heterocyclic group, and at this time, two or more of Rx, Ry, and Rz may be combined together to form a saturated or unsaturated hetero ring.

5. The compound according to claim 2, or a pharmaceutically acceptable salt thereof, wherein in Formula (I) or (II), Poly is a glycosaminoglycan residue.

6. The compound according to claim 2, or a pharmaceutically acceptable salt thereof, wherein in Formula (I) or (II), Poly is a residue of chondroitin, chondroitin sulfate or hyaluronic acid.

7. The compound according to claim 1, represented by the following Formula (XX), or a pharmaceutically acceptable salt thereof; 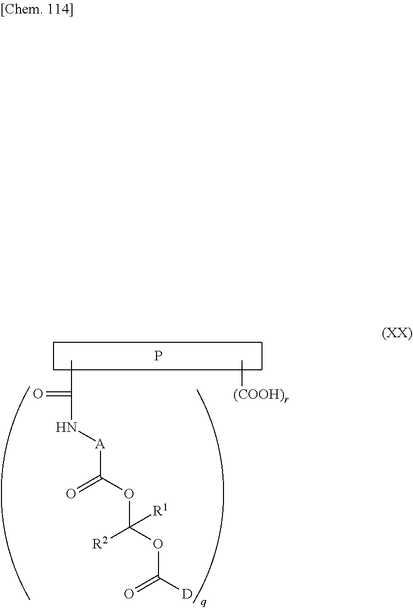
in Formula (XX), D, R¹, R², and A are as defined in claim 1, 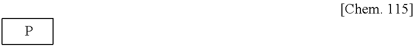
a moiety represented by P represents a polymer excluding a group derived from a carboxy group and the carboxy group of the acidic polysaccharide; q represents a number of a compound condensed with the polymer; r represents a number of substitution of the carboxy group.

8. The compound according to claim 7, or a pharmaceutically acceptable salt thereof, wherein the compound represented by the Formula (XX) is a compound represented by the following Formula (XXX): 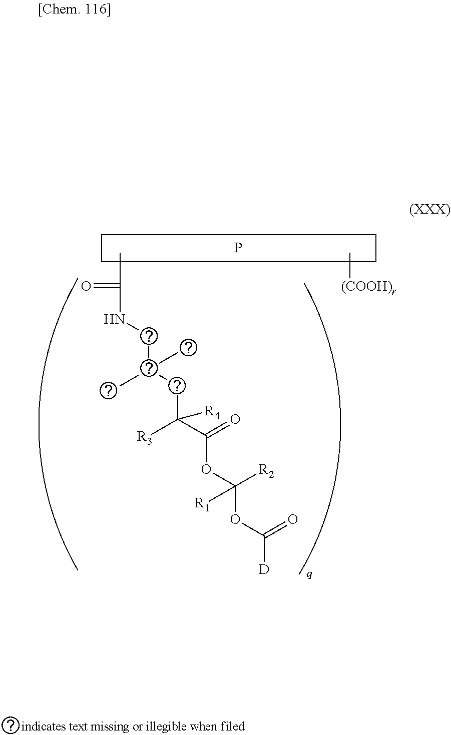
in Formula (XXX), D represents a residue of a primary or secondary amine compound DH excluding a hydrogen atom of a primary or secondary amino group, R¹, and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; R³, R⁴, R⁵, and R⁶ are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three groups of R¹, R², R³, R⁴, R⁵, and R⁶ may combine together to form a ring; 1 and n are each independently 0, 1, or 2, and m is 0 or 1; and q represents a number of a compound condensed with the polymer; r represents a number of substitution of the carboxy group, 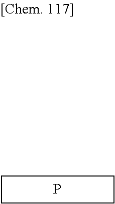
a moiety represented by P is a polymer excluding a group derived from a carboxy group and the carboxy group.

9. A method for producing a compound represented by the following Formula (I) or a pharmaceutically acceptable salt thereof, the method comprising a step of condensing a compound represented by the following Formula (III) and an acidic polysaccharide represented by the following Formula (IV): 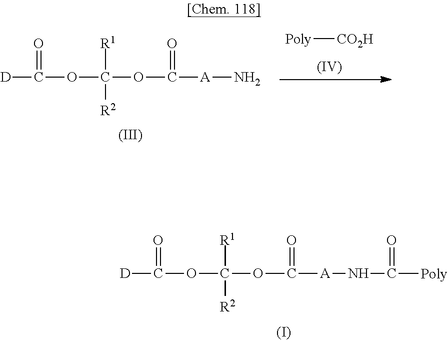
wherein, in Formulas (I), (III), and (IV), D represents a residue of a primary or secondary amine compound DH excluding a hydrogen atom of a primary or secondary amino group; R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a substituted or unsubstituted bivalent hydrocarbon group and may contain one or more hetero atoms at a position except for both ends which are bonded to —C(═O)— or —NH—, the hetero atoms are each independently selected from the group consisting of —O—, —NH— which may have a substituent, and —S—; any two or three groups of R¹, R², and A may combine together to form a ring; Poly represents an acidic polysaccharide residue and —C(═O)— adjacent to Poly is derived from a carboxy group of the acidic polysaccharide, and the compound represented by Formula (III) may form a salt with an inorganic acid or an organic acid.

10. A method for producing a conjugate, the method comprising a step of bonding a primary or secondary amine compound to an acidic polysaccharide via a linker represented by the following Formula (V): 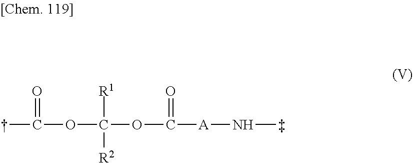
wherein, in the (V), R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; A is a substituted or unsubstituted bivalent hydrocarbon group and may contain one or more hetero atoms at a position except for both ends which are bonded to —C(═O)— or —NH— the hetero atoms are each independently selected from the group consisting of —O—, —NH— which may have a substituent, and —S—; any two or three groups of R¹, R², and A may combine together to form a ring; symbol † represents a node with a moiety of a nitrogen atom of an amino group of the primary or secondary amine compound, and symbol ‡ represents a node with a moiety of a carbonyl carbon derived from a carboxy group of the acidic polysaccharide.

11. The method for producing a conjugate according to claim 10, wherein the linker is represented by the following Formula (VI): 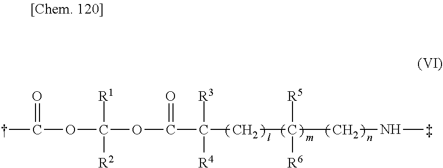
wherein, in the (VI), R¹ and R² are each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; R³, R⁴, R⁵, and R⁶ each independently a hydrogen atom, a substituted or unsubstituted alkyl group, a substituted or unsubstituted cycloalkyl group, a substituted or unsubstituted alkenyl group, a substituted or unsubstituted cycloalkenyl group, a substituted or unsubstituted alkynyl group, a substituted or unsubstituted aromatic group, or a substituted or unsubstituted heterocyclic group; any two or three groups of R¹, R², R³, R⁴, R⁵, and R⁶ may combine together to form a ring; 1 and n are each independently 0, 1, or 2, and m is 0 or 1; symbol t represents a node with a moiety of a nitrogen atom of an amino group of the primary or secondary amine compound, and symbol t represents a node with a moiety of a carbonyl carbon derived from a carboxy group of the acidic polysaccharide.
